SEQUENCE LISTING (38 sequences)

SEQ ID NO: 1 - protein, 756 aa, Homo sapiens [sequence omitted]

SEQ ID NO: 2 - protein, 544 aa, Homo sapiens
  misc_feature: Incyte ID No 7472835CD1
MAKKAIAVIGAGISGLGAIKCCLDEDLEPTCFERNDDIGHLWKFQKNTSEKMPSIYKSVTINTSKEMMCFSDFPVPDHFPNYMHNSKLMDYFGMYATHFGLLNYIRFKTEVQSVRKHPDFSINGQWDVVVETEEKQETLVFDGVLVCSGHHTDPYLPLQSFPGMEKFEGCYFHSREYKSPEDFSGKRIIVIGIGNSGVDIAVELSRVAKQVIFLSTRRGSWILHRVWDNGYPMDSSFFTRFNSFLQKILTTPQINNQLEKIMNSRFNHAHCGLQPQHRALSQHPTVSDDLPNHIISGKVQVKPSVKEFTETDAIFEDSTVEENIDVVIFATGYSFSFSFLDGLIKVTNNEVSLYKLMFPPDLEKPTLAVIGLIQPLGIILPIAELQSRWATRVFKGLIKLPSAENMMADIAQRKRAMEKRYVKTPRHTIQVDHIEYMDEIAMPAGVKPNLLFLFLSDPKLAMEVFFGPCTPYQYHLHGPEKWDGARRANLTQRERIIKPLRTRITSEDSHPSSQLSWIKMAPVSLAFLAAGLAYFRYTPYGKWK

SEQ ID NO: 3 - protein, 501 aa, Homo sapiens
  misc_feature: Incyte ID No 7476203CD1
MLSLLSGLALLAISFLLLKLGTFCWDRSCLPPGPLPFPILGNLWQLCFQQPHLSLKNFQKKIGNIFMNLGSSVVPLALPLLPVTFHPLNQGVLCKPLITFPKPFPTRNPGIICSSGHTWRQKRRFCLVMIRGLGLGKLALEVQLQKEAAELAEAFRQEQGKRPFDPQVSIVRSTVRVIGALVFGHHFLLEDPIFQELTQAIDFGLAFVSTVWRQLYDVFPWALCHLPGPHQEIFRYQEVVLSLIHQEITRHKLRAPEAPRDFISCYLAQISKAMDDPVSTFNQENLVQVVIDLFLGGTDTTATTLCWALIHMIQHGAVQETVQLELDEVLGAAPVVCYEDRKRLPYTMAVLHDVQRLSSVMAMGAVRQCVTSTRVCSYPVSKGTIILPNLASVLYDPECWETPRQFNPGHFSDKDGNFVANEAFLPFSAGTRVYPADQLAQMELFLMFATLLRTFRFQLPEGSPGLKLEYIFGGTWQPQPQEICAVPRLSSPSPGPREDGL

SEQ ID NO: 4 - protein, 345 aa, Homo sapiens
  misc_feature: Incyte ID No 7478583CD1
MKAAVWYGQKDVRVEEREPKELQDNEVKVKVSWAGICGTDLHEYLEGPIFISTEKPDPFLGQKAPVTLGHEFAGVVEETGSQVTKFNKGDRVVVNPTVSKREKEENIDLYDGYSFIGLGSDGGFAEFTNAPEENVYKLPDNVSDKEGALVEPTAVAVQAIKEGEVLFGDTVAIFGAGPIGLLTVVAAKAAGASKIFVFDLSEERLSKAKALGATHAINSGKTDPVDVINEYTENGVDVSFEVAGVAPTLKSSIDVTKARGTVVIVSIFGHPIEWNPMQLTNTGVKLTSTIAYTPTTFQQTIDLINEGNLNVKDVVTDEIELENIVESGFEQLVNDKSQAKILIKL

SEQ ID NO: 5 - protein, 361 aa, Homo sapiens
  misc_feature: Incyte ID No 7478585CD1
MSAQFENVQNPSITREDVAEVLVSVLTDETLQVVLAKRPQSIPQDDVFRFETIETREPHAGEVQVESIYVSVDPYMRGRMNDTKSYVQPFQVNEPLQGHIVGKVTQSNDERLSVGDYVTGILPWKKINTVNGDDVTPVPSKDVPLHLYLSVLGMPGMTAYTGLLQIGQPQSGETVVVSAASGAVGSVVGQIAKIKGAKVVGIAGGKQKTTYLTDELGFDAAIDYKQDDFAQQLEAAVPDGIDVYFENVGGVISDEVFKHLNRFARVPVCGAISAYNNEKDDIGPRIQGTLIKNQALMQGFVVAQFADHFKEASEQLAQWVSEGKIKFEVTIDEGFDNLPSAFRKLFTGDNFGKQVVKIKEE

SEQ ID NO: 6 - protein, 499 aa, Homo sapiens
  misc_feature: Incyte ID No 7479904CD1
MDEKSNKLLLALVMLFLFAVIVLQYVCPGTECQLLRLQAFSSPVPDPYRSEDESSARFVPRYNFTRGDLLRKVDFDIKGDDLIVFLHIQKTGGTTFGRHLVRNIQLEQPCECRVGQKKCTCHRPGKRETWLFSRFSTGWSCGLHADWTELTSCVPSVVDGKRDARLRPSRWRIFQILDAASKDKRGSPNTNAGANSPSSTKTRNTSKSGKNFHYITILRDPVSRYLSEWRHVQRGATWKASLHVCDGRPPTSEELPSCYTGDDWSGCPLKEFMDCPYNLANNRQVRMLSDLTLVGCYNLSVMPEKQRNKVLLESAKSNLKHMAFFGLTEFQRKTQYLFEKTFNMNFISPFTQYNTTRASSVEINEEIQKRIEGLNFLDMELYSYAKDLFLQRYQFMRQKEHQEARRKRQEQRKFLKGRLLQTHFQSQGQGQSQNPNQNQSQNPNPNANQNLTQNLMQNLTQSLSQKENRESPKQNSGKEQNDNTSNGTNDYIGSVEKWR

SEQ ID NO: 7 - protein, 222 aa, Homo sapiens
  misc_feature: Incyte ID No 7480367CD1
MAEKPKLHYSNARGSMESIRWLLAAAGVELEEKFLESAEDLDKLRNDGSLLFQQVPMVEIDGMKLVQTRAILNYIASKYNLYGKDMKERALIDMYTEGIVDLTEMILLLLICQPEERDAKTALVKEKIKNRYFPAFEKVLKSHRQDYLVGNKLSWADIHLVELFYYVEELDSSLISSFPLLKALKTRISNLPTVKKFLQPGSQRKPPMDEKSLEEARKIFRF

SEQ ID NO: 8 - protein, 330 aa, Homo sapiens
  misc_feature: Incyte ID No 8069390CD1
MAAVDSFYLLYREIARSCNCYMEALALVGAWYTARKSITVICDFYSLIRLHFIPRLGSRADLIKQYGRWAVVSGATDGIGKAYAEELASRGLNIILISRNEEKLQVVAKDIADTYKVETDIIVADFSSGREIYLPIREALKDKDVGILVNNVGVFYPYPQYFTQLSEDKLWDIINVNIAAASLMVHVVLPGMVERKKGAIVTISSGSCCKPTPQLAAFSASKAYLDHFSRALQYEYASKGIFVQSLIPFYVATSMTAPSNFLHRCSWLVPSPKVYAHHAVSTLGISKRTTGYWSHSIQFLFAQYMPEWLWVWGANILNRSLRKEALSCTA

SEQ ID NO: 9 - protein, 303 aa, Homo sapiens [sequence omitted]

SEQ ID NO: 10 - protein, 584 aa, Homo sapiens
  misc_feature: Incyte ID No 7478588CD1
MPSTVLPSTVLPSLLPTAGAGWSMRWILCWSLTLCLMAQTALGALHTKRPQVVTKYGTLQGKQMHVGKTPIQVFLGVPFSRPPLGILRFAPPEPPEPWKGIRDATTYPPGCLQESWGQLASMYVSTRERYKWLRFSEDCLYLNVYAPARAPGDPQLPVMVWFPGGAFIVGAASSYEGSDLAAREKVVLVFLQHRLGIFGFLSTDDSHARGNWGLLDQMAALRWVQENIAAFGGDPGNVTLFGQSAGAMSISGLMMSPLASGLFHRAISQSGTALFRLFITSNPLKVAKKVAHLAGCNHNSTQILVNCLRALSGTKVMRVSNKMRFLQLNFQRDPEEIIWSMSPVVDGVVIPDDPLVLLTQGKVSSVPYLLGVNNLEFNWLLPYIMKFPLNRQAMRKETITKMLWSTRTLLNITKEQVPLVVEEYLDNVNEHDWKMLRNRMMDIVQDATFVYATLQTAHYHRDAGLPVYLYEFEHHARGIIVKPRTDGADHGDEMYFLFGGPFATGLSMGKEKALSLQMMKYWANFARTGNPNDGNLPCWPRYNKDEKYLQLDFTTRVGMKLKEKKMAFWMSLYQSQRPEKQRQF

SEQ ID NO: 11 - protein, 508 aa, Homo sapiens [sequence omitted]

SEQ ID NO: 12 - protein, 439 aa, Homo sapiens
  misc_feature: Incyte ID No 3538709CD1
MLTGVTDGIFCCLLGTPPNAVGPLESVESSDGYTFVEVKPGRVLRVKHAGPAPAAAPPPPSSASSDAAQGDLSGLVRCQRRITVYRNGRLLVENLGRAPRADLLHGQNGSGEPPAALEVELADPAGSDGRLAPGSAGSGSGSGSGGRRRRARRPKRTIHIDCEKRITSCKGAQADVVLFFIHGVGGSLAIWKEQLDFFVRLGYEVVAPDLAGHGASSAPQVAAAYTFYALAEDMRAIFKRYAKKRNVLIGHSYGVSFCTFLAHEYPDLVHKVIMINGGGPTALEPSFCSIFNMPTCVLHCLSPCLAWSFLKAGFARQGAKEKQLLKEGNAFNVSSFVLRAMMSGQYWPEGDEVYHAELTVPVLLVHGMHDKFVPVEEDQRMAEILLLAFLKLIDEGSHMVMLECPETVNTLLHEFLLWEPEPSPKALPEPLPAPPEDKK

SEQ ID NO: 13 - protein, 514 aa, Homo sapiens [sequence omitted]

SEQ ID NO: 14 - protein, 226 aa, Homo sapiens
  misc_feature: Incyte ID No 7472027CD1
MRLCEKTELQLIGVPESDRENGTKLENTFQDIIQENFPNLARQANIQIQMAGGSIWIEGIPFPSNNFTDLRRLQDEIVLRDEDVITLSYPKSGSFWIVEIISLIHSKGDPSWVQSVVPWDRSPWIEVKRKKAGLESQKGPHLYTSHLPIQLFPKSFLNSKAKCIYPHVLMLVVLILGHKSQWSIAIKISENAEATSKLGNGQRLEEFGGLRRRQEDERSLEFLRDC

SEQ ID NO: 15 - protein, 121 aa, Homo sapiens [sequence omitted]

SEQ ID NO: 16 - protein, 486 aa, Homo sapiens
  misc_feature: Incyte ID No 1618256CD1
MGPLSPARTLRLWGPRSLGVALGVFMTIGFALQLLGGPFQRRLPGLQLRQPSAPSLRPALPSCPPRQRLVFLKTHKSGSSSVLSLLHRYGDQHGLRFALPARYQFGYPKLFQASRVKGYRPQGGGTQLPFHILCHHMRFNLKEVLQVMPSDSFFFSIVRDPAALARSAFSYYKSTSSAFRKSPSLAAFLANPRGFYRPGARGDHYARNLLWFDFGLPFPPEKRAKRGNIHPPRDPNPPQLQVLPSGAGPRAQTLNPNALIHPVSTVTDHRSQISSPASFDLGSSSFIQWGLAWLDSVFDLVMVAEYFDESLVLLADALCWGLDDVVGFMHNAQAGHKQGLSTVSNSGLTAEDRQLTARARAWNNLDWALYVHFNRSLWARIEKYGQGRLQTAVAELRARREALAKHCLVGGEASDPKYITDRRFRPFQFGSAKVLGYILRSGLSPQDQEECERLATPELQYKDKLDVKQFPPTVSLPLKTSRPLSP

SEQ ID NO: 17 - protein, 649 aa, Homo sapiens
  misc_feature: Incyte ID No 3387823CD1
MYISCLSLSLFFLSGPLQRVLEVSNHWWYSMLILPPLLKDSVAAPLLSAYYPDCVGMSPSCTSTNRAAATGNASPGKLEHSKAALSVHVPGMNRYFQPFYQPNECGKALCVRPDVMELDELYEFPEYSRDPTMYLALRNLILALWYTNCKEALTPQKCIPHIIVRGLVRIRCVQEVERILYFMTRKGLINTGVLSVGADQYLLPKDYHNKSVIIIGAGPAGLAAARQLHNFGIKVTVLEAKDRIGGRVWDDKSFKGVTVGRGAQIVNGCINNPVALMCEQLGISMHKFGERCDLIQEGGRITDPTIDKRMDFHFNALLDVVSEWRKDKTQLQDVPLGEKIEEIYKAFIKESGIQFSELEGQVLQFHLSNLEYACGSNLHQVSARSWDHNEFFAQFAGDHTLLTPGYSVIIEKLAEGLDIQLKSPVQCIDYSGDEVQVTTTDGTGYSAQKVLVTVPLALLQKGAIQFNPPLSEKKMKAINSLGAGIIEKIALQFPYRFWDSKVQGADFFGHVPPSASKRGLFAVFYDMDPQKKHSVLMSVIAGEAVASVRTLDDKQVLQQCMATLRELFKEQEVPDPTKYFVTRWSTDPWIQMAYSFVKTGGSGEAYDIIAEDIQGTVFFAGEATNRHFPQTVTGAYLSGVREASKIAAF

SEQ ID NO: 18 - protein, 258 aa, Homo sapiens
  misc_feature: Incyte ID No 55142051CD1
MSPAIALAFLPLVVTLLVRYRHYFRLLVRTVLLRSLRDCLSGLRIEERAFSYVLTHALPGDPGHILTTLDHWSSRCEYLSHMGPVKGQILMRLVEEKAPACVLELGTYCGYSTLLIARALPPGGRLLTVERDPRTAAVAEKLIRLAGFDEHMVELIVGSSEDVIPCLRTQYQLSRADLVLLAHRPRCYLRDLQLLEAHALLPAGATVLADHVLFPGAPRFLQYAKSCGRYRCRLHHTGLPDFPAIKDGIAQLTYAGPG

SEQ ID NO: 19 - protein, 544 aa, Homo sapiens
  misc_feature: Incyte ID No 7395274CD1
MSSPGPSQPPAEDPPWPARLLRAPLGLLRLDPSGGALLLCGLVALLGWSWLRRRRARGIPPGPTPWPLVGNFGHVLLPPFLRRRSWLSSRTRAAGIDPSVIGPQVLLAHLARVYGSIFSFFIGHYLVVVLSDFHSVREALVQQAEVFSDRPRVPLISIVTKEKGVVFAHYGPVWRQQRKFSHSTLRHFGLGKLSLEPKIIEEFKYVKAEMQKHGEDPFCPFSIISNAVSNIICSLCFGQRFDYTNSEFKKMLGFMSRGLEICLNSQVLLVNICPWLYYLPFGPFKELRQIEKDITSFLKKIIKDHQESLDRENPQDFIDMYLLHMEEERKNNSNSSFDEEYLFYIIGDLFIAGTDTTTNSLLWCLLYMSLNPDVQEKVHEEIERVIGANRAPSLTDKAQMPYTEATIMEVQRLTVVVPLAIPHMTSENTVLQGYTIPKGTLILPNLWSVHRDPAIWEKPEDFYPNRFLDDQGQLIKKETFIPFGIGKRVCMGEQLAKMELFLMFVSLMQSFAFALPEDSKKPLLTGRFGLTLAPHPFNITISRR

SEQ ID NO: 20 - DNA, 2603 bp, Homo sapiens
  misc_feature: Incyte ID No 7248285CB1
atggcgtggtccccaccagccaccctctttctgttcctgctgctgctaggccagccccctcccagcaggccacagtcactgggcaccactaagctccggctggtgggcccagagagcaagccagaggagggccgcctggaggtgctgcaccagggccagtggggcaccgtgtgtgatgacaactttgctatccaggaggccacagtggcttgccgccagctgggcttcgaagctgccttgacctgggcccacagtgccaagtacggccaaggggagggacccatctggctggacaatgtgcgctgtgtgggcacagagagctccttggaccagtgcgggtctaatggctggggagtcagtgactgcagtcactcagaagacgtaggggtgatatgccacccccggcgccatcgtggctacctttctgaaactgtctccaatgcccttgggccccagggccagcggctggaggaggtgcggctcaagcccatccttgccagtgccaagcagcatagcccagtgaccgagggagccgtggaggtgaagtatgagggccactggcggcaggtgtgtgaccagggctggaccatgaacaacagcagggtggtgtgcgggatgctgggcttccccagcgaggtgcctgtcgacagccactactacaggaaagtctgggatctgaagatgagggaccctaagtctaggctgaagagcctgacgaataagaactccttctggatccaccaggtcacctgcctggggacagagccccacatggccaactgccaggtgcaggtggctccagcccggggcaagctgcggccagcctgcccaggtggcatgcatgctgtggtcagctgtgtggcagggcctcacttccgcccaccgaagacaaagccacaacgcaaagggtcctgggcagaggagccgagggtgcgcctgcgctccggggcccaggtgggcgagggccgggtggaagtgctcatgaaccgccagtggggcacggtctgtgaccacaggtggaacctcatctctgccagtgtcgtgtgtcgtcagctgggctttggctctgctcgggaggccctctttggggcccggctgggccaagggctagggcccatccacctgagtgaggtgcgctgcaggggatatgagcggaccctcagcgactgccctgccctggaagggtcccagaatggttgccaacatgagaatgatgctgctgtcaggtgcaatgtccctaacatgggctttcagaatcaggtgcgcttggctggtgggcgtatccctgaggaggggctattggaggtgcaggtggaggtgaacggggtcccacgctgggggagcgtgtgcagtgaaaactgggggctcaccgaagccatggtggcctgccgacagctcggcctgggttttgccatccatgcctacaaggaaacctggttctggtcggggacgccaagggcccaggaggtggtgatgagtggggtgcgctgctcaggcacagagctggccctgcagcagtgccagaggcacgggccggtgcactgctcccacggtggcgggcgcttcctggctggagtctcctgcatggacagtgcaccagacctggtgatgaacgcccagctagtgcaggagacggcctacttggaggaccgcccgctcagccagctgtattgtgcccacgaggagaactgcctctccaagtctgcggatcacatggactggccctacggataccgccgcctattgcgcttctccacacagatctacaatctgggccggactgactttcgtccaaagactggacgcgatagctgggtttggcaccagtgccacaggcattaccacagcattgaggtcttcacccactacgacctcctcactctcaatggctccaaggtggctgaggggcacaaggccagcttctgtctggaggacacaaactgccccacaggactgcagcggcgctacgcatgtgccaactttggagaacagggagtgactgtaggctgctgggacacctaccggcatgacattgattgccagtgggtggatatcacagatgtgggccccgggaattatatcttccaggtgattgtgaacccccactatgaagtggcagagtcagatttctccaacaatatgctgcagtgccgctgcaagtatgatgggcaccgggtctggctgcacaactgccacacagggaattcatacccagccaatgcagaactctccctggagcaggaacagcgtctcaggaacaacctcatctgaagctgtcactgcacactcctagctgctgccgatacaccagatacctcagcttattggagccatgcccttcacagagtcccaactcagaggaaaagggccagtgccaaggggcaccaagaacctgctcaggaagccttttgatggcaagatcaccaatccagatggtattgctccctcaggatggctctgggcctgcccctaagggcctgtggcctatggaatatgtcctccaggctttgctcagctgagctcctcttctgtaaggaaacccagtcatccctgaatcttgccacagagatccgggattcaggagctctcagtttcttaaggag

SEQ ID NO: 21 - DNA, 1745 bp, Homo sapiens
  misc_feature: Incyte ID No 7472835CB1
gtaattttttttaaatgggatggatgacagtgacagagctctaaatttctactaaccagctgagacacaatggccaaaaaagcgattgctgtgattggagctggaattagcggactgggggccatcaagtgctgcctggatgaagatctggagcccacctgctttgaaagaaatgatgatattggacatctctggaaatttcaaaaaaatacttcagagaaaatgcctagtatctacaaatctgtgaccatcaatacttccaaggagatgatgtgcttcagtgacttccctgtccctgatcattttcccaactacatgcacaactccaaactcatggactacttcgggatgtatgccacacactttggcctcctgaattacattcgttttaagactgaagtgcaaagtgtgaggaagcacccagatttttctatcaatggacaatgggatgttgttgtggagactgaagagaaacaagagactttggtctttgatggggtcttagtttgcagtggacaccacacagatccctacttaccacttcagtccttcccaggtatggagaaatttgaaggctgttatttccatagtcgggaatacaaaagtcccgaggacttttcagggaaaagaatcatagtgatcggcattggaaattctggagtggatattgcggtggagctcagtcgtgtagcaaaacaggttatattccttagtactagacgtggatcatggattttacaccgtgtttgggataatgggtatcccatggatagttcatttttcactcggttcaatagttttctccagaaaatactaactacaccacaaataaataaccagctagagaaaataatgaactcaagatttaatcatgcgcactgtggcctgcagcctcagcacagagctttaagtcagcatccaactgtcagtgatgacctgccaaatcacataatttctggaaaagtccaagtaaagcccagcgtgaaggagttcacagaaacagatgccatttttgaagacagcactgtagaggagaatattgatgttgtcatctttgctacaggatacagtttttctttttctttccttgatggtctgatcaaggttactaacaatgaagtatctctgtataagcttatgttccctcctgacctggagaagccaaccttggctgtcatcggtcttatccaaccactgggcatcatcttacctattgcagagctccaatctcgttgggctacacgagtgttcaaaggtctgatcaaattaccctcagcggagaacatgatggcagatattgcccagaggaaaagggctatggaaaaacggtatgtaaagacaccccgccacacaatccaagtggatcacattgagtacatggatgagattgccatgccagcaggggtgaaacccaacctgctcttcctctttctctcagatccaaagctggccatggaggttttctttggcccctgcaccccataccagtaccacctccatgggcccgagaaatgggatggggcccggagagctaacctgacccagagagagaggatcatcaagcccctgaggactcgcattactagtgaggacagccacccatcctcacagctctcttggataaagatggccccagtgagcctggcatttctggctgctggcttggcatactttcgatatactccttacggtaaatggaaataaatgaaagaacactgagggggaaaagcatggaatagtttcta

SEQ ID NO: 22 - DNA, 1587 bp, Homo sapiens
  misc_feature: Incyte ID No 7476203CB1
atgctctccctgctcagtgggctggctttactggccatctccttcctgctcctgaaacttggcaccttctgctgggacaggagctgtcttcctcctggcccactccccttccccatccttggaaacctgtggcagctatgctttcagcagcctcacctttcacttaaaaactttcagaagaagataggaaatatctttatgaacttgggaagcagtgtggttccactggcattgccattgttaccggtgactttccatcccttgaaccaaggcgtgttatgtaagccacttattactttccctaaacccttccctaccagaaatccaggcatcatctgcagcagcgggcacacgtggcggcaaaagagacgcttctgcctggtgatgattcgagggctgggcctaggcaagctggcgctggaggtgcagctgcagaaagaggcagcagagctggcagaagccttccgccaggagcagggtaagagacccttcgaccctcaggtatccattgtcaggtccacagtcagagtcatcggggcccttgtgtttggccaccacttcctcttagaggatcccatcttccaggaactgactcaagccatcgactttggcctggcatttgtcagcactgtgtggcgccagctgtatgacgtgtttccctgggccctctgccacctcccaggaccccaccaggagatatttaggtaccaagaggtcgtgctgagcttaatccaccaggagatcaccaggcacaaactcagggcaccggaggcccccagggacttcatcagctgctacctggcccagatctccaaggccatggatgaccctgtctccacattcaaccaggagaacctggtccaggtggtgatcgacctgtttctgggaggcaccgacaccacagccaccaccctgtgctgggcactcatccacatgatccagcacggagctgtccaggagacggtgcagctggagctggacgaggtgctgggtgctgccccggttgtctgctatgaagaccgcaagcgactgccttacaccatgctgtcctccatgacgtgcagcgcctcagcagcgtcatggccatgggtgccgtgcgccagtgtgtgacctccacccgtgtgtgcagctatcccgtgagcaagggcaccatcatcttacccaacctggcctctgtgctctatgaccctgagtgctgggagacccctcgacagttcaaccctggccacttctcggacaaggatggaaactttgtggccaatgaggccttcctgccattctctgcaggtactagggtctacccagcagaccagctggctcaaatggagctcttcctgatgtttgccaccctcctcaggacctttcggttccaactgccagaagggagcccggggctcaagctggagtacatctttggcggcacttggcaaccccagccccaggagatctgcgcagtgccccgcctgagcagccccagccctggtcctagggaggatggcctgtagccactgggggtctggaggcctgtcccccatgaagtccttcctcagtctcttttggttcctgcaaagttagaaaagaggagga

SEQ ID NO: 23 - DNA, 1038 bp, Homo sapiens
  misc_feature: Incyte ID No 7478583CB1
atgaaagcagcagtttggtatggtcaaaaagatgtaagagttgaagaacgtgaacctaaagaattacaggacaatgaagttaaggttaaagtatcttgggctggcatttgtggtacagatttacatgaatacttagaaggccctatatttatttcaacagaaaagccagatccattcttaggtcaaaaagcgccagttacattgggtcatgaatttgcaggtgtagtagaagaaactggttcccaagttacaaaatttaataaaggcgatcgagttgtagttaaccctacagtttcaaaacgtgaaaaagaagaaaatattgacctttatgatggttattcatttataggcttaggttctgatggtggatttgcagagtttacaaatgcgccggaagaaaatgtttataaactaccagataatgtttctgataaagaaggtgcgcttgtcgaaccaacagccgttgcagttcaagcaattaaagaaggtgaagttctatttggtgatactgtagctatttttggtgcaggaccaattggattattaacagtcgtagcagccaaagcagctggtgcaagtaaaatatttgttttcgatttatcagaagaaagactaagtaaagctaaagcactaggcgcaactcatgctataaactctggtaagacagatccagttgatgttattaatgagtatacagaaaatggtgtagatgtatcttttgaagtggctggtgtagcaccaacacttaaatcttctatagatgttacaaaagcaagaggtacagttgttatcgtttctatttttggtcatcctatcgagtggaatccaatgcaattaactaatacaggagtaaaacttacttctacaattgcatacacacctactacattccaacaaacaattgacttaatcaacgaaggtaatttaaacgttaaagatgtagttactgatgaaattgagttagaaaatatcgtagaatcaggatttgaacaacttgtaaatgataaatctcaagcaaaaatattaattaaattataa

SEQ ID NO: 24 - DNA, 1584 bp, Homo sapiens
  misc_feature: Incyte ID No 7478585CB1
atgaaatcattaatgattggcgctaatggtggcgtcggtcaacatctcgtacgtaaactaaatgcacgagatgttgattttacggccggtgttagaaaagaagaacaagttgaagctttaaaagcagatggtatcgatgcaacttacattgatgttgcaaaacaatctattgatgaattaatagaattatttaaaccgtatgaccaaatccttttttctgtcggttctggtggaagcacaggtgacgatcaaacaatcatagtagatttagacggttcagtgaaagcaattaaagcaagtgaacacgtcggtcgtcaacactttgttatggtatcaacgtacgattcacgtcgagaagcttttgatgcgtcaggcgacttgaaaccatacaccattgctaaacattatgcggatgactacttaagacatgcaaatttaaaatataccatcgtacatccaggcgcattaactaacaatcatgaaacgcaacaattcaatatgagtgctcaatttgaaaatgtacaaaatccgtctatcacgagagaagatgtagcagaagtgcttgtttctgtgttaactgatgaaacattacaagttgtacttgcaaaacgaccacaaagtatccctcaagacgatgtatttagatttgaaacaatagaaactcgagaaccacatgcaggtgaggttcaagtagaatccatttatgtatctgtagatccttacatgagaggcagaatgaatgatacaaaaagttatgttcaacctttccaagtgaatgaaccattacaaggtcatattgttggaaaagtcacacaatcgaacgatgaacgtctatctgttggcgattatgtcacaggcatattaccatggaaaaagataaatacagtgaatggagacgatgtgacccctgtgccatcaaaagatgtaccattacatttatatttgagtgttttaggcatgccgggaatgacggcatatacaggattgcttcaaattggtcaaccacaatctggcgagacggttgtcgtgtcagctgcatcaggtgcagtaggatctgtcgtaggacaaattgctaagattaaaggcgcaaaagttgtcggtattgctggtggtaagcagaaaacaacatatttaacagatgaattaggatttgatgcggccattgactataaacaagatgatttcgcacagcaactcgaagcggctgtaccagatggtattgatgtgtattttgaaaatgtaggcggcgtaatttctgatgaagtgtttaaacacttaaatcgatttgcacgcgttccggtatgtggtgcaatttcagcatataataatgaaaaagacgatattggaccacgtatccaaggaacgttgattaaaaatcaagcattgatgcaaggttttgtagtagcacaattcgctgatcattttaaagaagcaagcgaacaactcgcacaatgggtgtctgaaggtaaaattaaatttgaagtgacgatagatgaaggttttgacaatttaccttctgcattcagaaagttatttacaggtgataatttcggtaaacaagttgtcaaaatcaaagaagaatag

SEQ ID NO: 25 - DNA, 1500 bp, Homo sapiens
  misc_feature: Incyte ID No 7479904CB1
atggatgagaaatccaacaagctgctgctagctttggtgatgctcttcctatttgccgtgatcgtcctccaatacgtgtgccccggcacagaatgccagctcctccgcctgcaggcgttcagctccccggtgccggacccgtaccgctcggaggatgagagctccgccaggttcgtgccccgctacaatttcacccgcggcgacctcctgcgcaaggtagacttcgacatcaagggcgatgacctgatcgtgttcctgcacatccagaagaccgggggcaccactttcggccgccacttggtgcgtaacatccagctggagcagccgtgcgagtgccgcgtgggtcagaagaaatgcacttgccaccggccgggtaagcgggaaacctggctcttctccaggttctccacgggctggagctgcgggttgcacgccgactggaccgagctcaccagctgtgtgccctccgtggtggacggcaagcgcgacgccaggctgagaccgtccaggtggaggatttttcagattctagatgcagcaagtaaggataaacggggttctccaaacactaacgcaggcgccaactctccgtcatccacaaagacccggaacacatctaagagtgggaagaacttccactacatcaccatcctccgagacccagtgtcccggtacttgagtgagtggaggcatgtccagagaggggcaacatggaaagcatccctgcatgtctgcgatggaaggcctccaacctccgaagagctgcccagctgctacactggcgatgactggtctggctgccccctcaaagagtttatggactgtccctacaatctagccaacaaccgccaggtgcgcatgctctccgacctgaccctggtaggctgctacaacctctctgtcatgcctgaaaagcaaagaaacaaggtccttctggaaagtgccaagtcaaatctgaagcacatggcgttcttcggcctcactgagtttcagcggaagacccaatatctgtttgagaaaaccttcaacatgaactttatttcgccatttacccagtataataccactagggcctctagtgtagagatcaatgaggaaattcaaaagcgtattgagggactgaattttctggatatggagttgtacagctatgccaaagacctttttttgcagaggtatcagtttatgaggcagaaagagcatcaggaggccaggcgaaagcgtcaggaacaacgcaaatttctgaagggaaggctccttcagacccatttccagagccagggtcagggccagagccagaatccgaatcagaatcagagtcagaacccaaatccgaatgccaatcagaacctgactcagaatctgatgcagaatctgactcagagtttgagccagaaggagaaccgggaaagcccgaagcagaactcaggcaaggagcagaatgataacaccagcaatggcaccaacgactacataggcagtgtagagaaatggcgttaa

SEQ ID NO: 26 - DNA, 669 bp, Homo sapiens
  misc_feature: Incyte ID No 7480367CB1
atggcagagaagcccaagctccactactccaatgcacggggcagtatggagtccattcggtggctcctggctgcagctggagtagagttggaagagaaatttctagaatctgcagaagatttggacaagttaagaaatgatgggagtttgctgttccagcaagtaccaatggttgagattgacgggatgaagctggtgcagaccagagccattcttaactacattgccagcaaatacaacctttatgggaaagacatgaaggagagagccctgattgatatgtacacagaaggtatagtagatttgactgaaatgatccttcttctgctcatatgtcaaccagaggaaagagatgccaagactgccttggtcaaagagaaaataaaaaatcgctacttccctgcctttgaaaaagtattaaagagccacagacaagactaccttgttggcaacaagctgagctgggctgacattcacctggtggaacttttctactacgtggaagagcttgactcgagtcttatctccagcttccctctgctgaaggccctgaaaaccagaatcagcaacctgcccacggtgaagaagtttctgcagcctggcagccagagaaagcctcccatggatgagaaatctttagaagaagcaaggaagattttcaggttttaa

SEQ ID NO: 27 - DNA, 3551 bp, Homo sapiens
  misc_feature: Incyte ID No 8069390CB1
ggcgcggagcagctccggcggcgagacgggggcggcgccgcgcgggtctggcgggaccggtttggaagactttgccggcctgcagattggccttaagagaaggacggagccacatactgctgacggcccagaactggcagagagaaggttgccatggctgctgttgacagtttctacctcttgtacagggaaatcgccaggtcttgcaattgctatatggaagctctagctttggttggagcctggtatacggccagaaaaagcatcactgtcatctgtgacttttacagcctgatcaggctgcattttatcccccgcctggggagcagagcagacttgatcaagcagtatggaagatgggccgttgtcagcggtgcaacagatgggattggaaaagcctacgctgaagagttagcaagccgaggtctcaatataatcctgattagtcggaacgaggagaagttgcaggttgttgctaaagacatagccgacacgtacaaagtggaaactgatattatagttgcggacttcagcagcggtcgtgagatctaccttccaattcgagaagccctgaaggacaaagacgttggcatcttggtaaataacgtgggtgtgttttatccctacccgcagtatttcactcagctgtccgaggacaagctctgggacatcataaatgtgaacattgccgccgctagtttgatggtccatgttgtgttaccgggaatggtggagagaaagaaaggtgccatcgtcacgatctcttctggctcctgctgcaaacccactcctcagctggctgcattttctgcttctaaggcttatttagaccacttcagcagagccttgcaatatgaatatgcctctaaaggaatctttgtacagagtctaatccctttctatgtagccaccagcatgacagcacccagcaactttctgcacaggtgctcgtggttggtgccttcgccaaaagtctatgcacatcatgctgtttctactcttgggatttccaaaaggaccacaggatattggtcccattctattcagtttctttttgcacagtatatgcctgaatggctctgggtgtggggagcaaatattctcaaccgttcactacgtaaggaagccttatcctgcacagcctgagtctggatggccacttgagaagttttgccaactcctgggaacctcgatattctgacatttggaaaaacacatttaatttatctcctgtgtttcattgctgattattcagcatactgttgattcgtcatttgcaaaacacacataataccgtcagagtgctgtgaaaaaccttaagggtgtgtggatggcacaggatcaataatgcctgaggctgattgacgacatctacatttcagtgctttttccctaagctgtttgaaagttacgcttttctgttgttctagagccacagcagtctaatattgaaatataatatgatttgtcaggtcttataatttcagatgttgttttttaagggaaattgaccatttcactagaggagttgtgctggtttttaaatgtgcatcaagaaagactactgaaaagtattattttgtaactaagattgctggtactattaggaaaaatctgtgtgtattgtatagctctagctgtttgactatctgtaatgaaaatgctgcacttcaactggtatttcattagagaaccgtgtgtgtgcgtgtgtgtggtgcctttgagcaactttatttatggttaccatatttttaaaaagattttttgtcagggtgacttaacatggactcttatagggtattaaaacaatctagattattccttttcatcctaaataagcctaccaaatttcatgctgttggtttgccatgaatgatattacttcctacattatatttgtgttttttcaaatctgctatggaatgaacttattcctagatttggatatgtaagagaaacctgcagtcatcttttgatttataaggcaattcttgtggataaatagtgatttctcagcctctgacccattttataactgaaatttagccctttagagcttgttatatctggttttcctacgtttttctatgtaatattattccattccagtagcattattgatagaaatagtaagtatttatggaatagtaaaatatggacaaattacgtgtgtgacatatctgtcaaaataagttagaagcttattcttggtttgtgtaatgaatttatgtattgtagtgaatacctttactggtgtgaagataattatgcacaaaccctcacaatacgcgttaacattgaaacctgtgaaatgtccttaggttgggtcatataaagccaaccatttttgaggaccatgtacctagtgctttgaaaactgtaagtcactatatgaatatgacaatatgtgcacatttaaaattcagagctcggcattgtgatactgatgcagaagctagtagattggttaaaagtctggacttctgtggcatttttttcgtgacgtgataatctatcataagcagacctaagcacagttttatgaacacaattttgcccatgacattgcctacaggatttccagatgtgacttgcactcagaagatcagtggtcaacttcagaagttcttccacgcttagatcatgtcttcagaacttagatgtgaaaatctacacactgggagatgctgtgagccccaaggttttgatggagtttgcttggaatcctcttgacttcatgccacattgacgtgaactttgatgtataataagcagcagcaacttcatgtgaaaatatggtcaggtagttatatgtaaggttacgtggtccagtaatgtcttagattgataaattaggtatggaatccatcagtgttacgtgatgagaataggtgaacacaccttgtcagtgatgatgtaaacttctctccttggcaggacatgggcaaacatgctgattggtgcaaatgtggtgccgagctgtccatagctgcagtgaaagatgaagagcaagaccttctctaggttttctagctttcattaaatgtatttttttccccagagctaatttgaaagttgattggaccactgtggatggggtctcattaagaatgtgggaaataggggccgagtgcggtggctcacacctgtaatcccagcagtttggaaggccagggcaggtggatcgcttgatcccaggaggtcgagaccagcctggggaacacatcctgtctctacaaaaaatacaaaaattagccaggcagggtggtgcatgcctgtagtcccagctacttgggaggctgaggcaggagaattttttgagcccaggatgcagaggttgaagtgagccaagatcgtgccactgcactccagccttgagacagagcgagaccctgtctcaaaaaaaaaaaaagaacgtgggaaatatgaacctttgaaagttaatctgtgaattgaaagtttaacaataaaagtagttgtttgtttcctttgaaaaaaaaaaaaaaaaaaaaaaaaaaaaaaaaaaaaaaaaaatggcggtcgcaagctta

SEQ ID NO: 28 - DNA, 2178 bp, Homo sapiens
  misc_feature: Incyte ID No 7473869CB1
tcgggtggggagtagagtaggtaagcgtgtttctttattggcggagggtatgtgtaaccgagagggtttcactgccaattatggaagtagcctctccttgatattgttgaggtgggattgtgcaacaacattgtactgttgtgggttgtaatttcgcctagatggcataagtgagaactgaagacttcaggtaggatcagaggcacgggtcttgttagttatgctcttgggatcagtgtcgcctgctgagctgaaaaggaaatggattcaatctcttcaacctttaaggtgatagatagtttgagcagactggagaatggacacactatgaagctgtggctagaaagggactggtcatgtcccatcctctggccagattgactggggatgtccggacagatgcctgcatgggtggtgagggccacatctgcacacgagccagtggctgcttgcagttcactgctgtgatgccagagtgtgttcaaaggtgactctcctgctcttctggactcttctctcaggcaagaaaggctgcaggctgcctgctatgtgatgcctgagcacaaagccaaggaactgaactaagtctttctgttaagtcctgagtttgtcattggcaggtttacttgtggccagctctctctgcccttgggggttccgtcttctcactgcggaccctggattgaaacgatctccccgcggccgccgccgctacctggtgcccgcaggtgcctgcaggagtcctggggccagctggcctcgatgtacgtcagcacgcgggaacggtacaagtggctgcgcttcagcgaggactgtctgtacctgaacgtgtacgcgccggcgcgcgcgcccggggatccccagctgccagtgatggtctggttcccgggaggcgccttcatcgtgggcgctgcttcttcgtacgagggctctgacttggccgcccgcgagaaagtggtgctggtgtttctgcagcacaggctcggcatcttcggcttcctgagcacggacgacagccacgcgcgcgggaactgggggctgctggaccagatggcggctctgcgctgggtgcaggagaacatcgcagccttcgggggagacccaggaaatgtgaccctgttcggccagtcggcgggggccatgagcatctcaggactgatgatgtcacccctagcctcgggtctcttccatcgggccatttcccagagtggcaccgcgttattcagacttttcatcactagtaacccactgaaagtggccaagaaggttgcccacctggctggatgcaaccacaacagcacacagatcctggtaaactgcctgagggcactatcagggaccaaggtgatgcgtgtgtccaacaagatgagattcctccaactgaacttccagagagacccggaagagattatctggtccatgagccctgtggtggatggtgtggtgatcccagatgaccctttggtgctcctgacccaggggaaggtttcatctgtgccctaccttctaggtgtcaacaacctggaattcaattggctcttgccttatatcatgaagttcccgctaaaccggcaggcgatgagaaaggaaaccatcactaagatgctctggagtacccgcaccctgttggtgagggacccagctggcaggggtgctcagttcggacagggttgacccccctgttttttttaacctagtagctgctctttgcaaaggggctcccagccagggtaaggatctttcttggagggtctggggtttgctgtgggatcagatgactgcttacaggtaaggtgctcagggtcacaggggcagttatgcagcaaaatcaggggttacaatcagcagagacagaaactttcccaggaagctccctttctccccctcccaggccaaaaactcctggggggctgagcatggatccaagtcactggtgggcccacctctggcccagctggcacccaggcctcaggtaagtgtggcctcctcactcagaatatcaccaaggagcaggtaccacttgcggtggaggagtacctggacaatgtcaatgagcatgactggaagatgctacgaaaccgtatgatggacatagttcaagatgccactttcgtgtatgccacactgcagactgctcactaccaccgagatgccggccttcctgtctacctgtatgaa

SEQ ID NO: 29 - DNA, 2081 bp, Homo sapiens
  misc_feature: Incyte ID No 7478588CB1
gctgtgggctggtcagaagctggttacaattccccccgccccagtacttgctggcagggattaagagcagataaaagtgtgctcacacactgtagacacggctaccatgccatccacagtgttgccatccacagtgttgccatcactcctgcccacagcaggagctggctggagcatgaggtggattctgtgctggagcctcaccctctgcctgatggcgcagacggccttgggtgccttgcacaccaagaggcctcaagtggtcaccaaatatggaaccctgcaaggaaaacagatgcatgtggggaagacacccatccaagtctttttaggagtccccttctccagacctcctctaggtatcctcaggtttgcacctccagaacccccggagccctggaaaggaatcagagatgctaccacctacccgcctgggtgcctgcaggagtcctggggccagctggcctcgatgtacgtcagcacgcgggaacggtacaagtggctgcgcttcagcgaggactgtctgtacctgaacgtgtacgcgccggcgcgcgcgcccggggatccccagctgccagtgatggtctggttcccgggaggcgccttcatcgtgggcgctgcttcttcgtacgagggctctgacttggccgcccgcgagaaagtggtgctggtgtttctgcagcacaggctcggcatcttcggcttcctgagcacggacgacagccacgcgcgcgggaactgggggctgctggaccagatggcggctctgcgctgggtgcaggagaacatcgcagccttcgggggagacccaggaaatgtgaccctgttcggccagtcggcgggggccatgagcatctcaggactgatgatgtcacccctagcctcgggtctcttccatcgggccatttcccagagtggcaccgcgttattcagacttttcatcactagtaacccactgaaagtggccaagaaggttgcccacctggctggatgcaaccacaacagcacacagatcctggtaaactgcctgagggcactatcagggaccaaggtgatgcgtgtgtccaacaagatgagattcctccaactgaacttccagagagacccggaagagattatctggtccatgagccctgtggtggatggtgtggtgatcccagatgaccctttggtgctcctgacccaggggaaggtttcatctgtgccctaccttctaggtgtcaacaacctggaattcaattggctcttgccttatatcatgaagttcccgctaaaccggcaggcgatgagaaaggaaaccatcactaagatgctctggagtacccgcaccctgttgaatatcaccaaggagcaggtaccacttgtggtggaggagtacctggacaatgtcaatgagcatgactggaagatgctacgaaaccgtatgatggacatagttcaagatgccactttcgtgtatgccacactgcagactgctcactaccaccgagatgccggcctccctgtctacctgtatgaatttgagcaccacgctcgtggaataatcgtcaaaccccgcactgatggggcagaccatggggatgagatgtacttcctctttgggggccccttcgccacaggcctttccatgggtaaggagaaggcacttagcctccagatgatgaaatactgggccaactttgcccgcacaggaaaccccaatgatgggaatctgccctgctggccacgctacaacaaggatgaaaagtacctgcagctggattttaccacaagagtgggcatgaagctcaaggagaagaagatggctttttggatgagtctgtaccagtctcaaagacctgagaagcagaggcaattctaagggtggctatgcaggaaggagccaaagaggggtttgcccccaccatccaggccctggggagactagccatggacatacctggggacaagagttctacccaccccagtttagaactgcaggagctccctgctgcctccaggccaaagctagagcttttgcctgttgtgtgggacctgcactgccctttccagcctgacatcccatgatgcccctctacttc

SEQ ID NO: 30 - DNA, 2642 bp, Homo sapiens
  misc_feature: Incyte ID No 55046125CB1
gagccggcccgggtcggtcgccgcctcagttcgcgcgggccctcctaggggtgtgtctcacggattccacacccggccgctcctggacaagccccgaaaggcgtcttcttccctggcgggagccgcgtgcgccccgcttttcgcgctgctgtcccggggccgccgcaggcggatgcacgtcctcaggcgacgctgggacctgggctccctctgccgggccctgctcactcggggcctggccgccctgggccactcgctgaagcacgtgctcggtgcgatcttctccaagattttcggccccatggccagcgtcgggaacatggatgagaaatccaacaagctgctgctagctttggtgatgctcttcctatttgccgtgatcgtcctccaatacgtgtgccccggcacagaatgccagctcctccgcctgcaggcgttcagctccccggtgccggacccgtaccgctcggaggatgagagctccgccaggttcgtgccccgctacaatttcacccgcggcgacctcctgcgcaaggtagacttcgacatcaagggcgatgacctgatcgtgttcctgcacatccagaagaccgggggcaccactttcggccgccacttggtgcgtaacatccagctggagcagccgtgcgagtgccgcgtgggtcagaagaaatgcacttgccaccggccgggtaagcgggaaacctggctcttctccaggttctccacgggctggagctgcgggttgcacgccgactggaccgagctcaccagctgtgtgccctccgtggtggacggcaagcgcgacgccaggctgagaccgtccaggaacttccactacatcaccatcctccgagacccagtgtcccggtacttgagtgagtggaggcatgtccagagaggggcaacatggaaagcatccctgcatgtctgcgatggaaggcctccaacctccgaagagctgcccagctgctacactggcgatgactggtctggctgccccctcaaagagtttatggactgtccctacaatctagccaacaaccgccaggtgcgcatgctctccgacctgaccctggtaggctgctacaacctctctgtcatgcctgaaaagcaaagaaacaaggtccttctggaaagtgccaagtcaaatctgaagcacatggcgttcttcggcctcactgagtttcagcggaagacccaatatctgtttgagaaaaccttcaacatgaactttatttcgccatttacccagtataataccactagggcctctagtgtagagatcaatgaggaaattcaaaagcgtattgagggactgaattttctggatatggagttgtacagctatgccaaagacctttttttgcagaggtatcagtttatgaggcagaaagagcatcaggaggccaggcgaaagcgtcaggaacaacgcaaatttctgaagggaaggctccttcagacccatttccagagccagggtcagggccagagccagaatccgaatcagaatcagagtcagaacccaaatccgaatgccaatcagaacctgactcagaatctgatgcagaatctgactcagagtttgagccagaaggagaaccgggaaagcccgaagcagaactcaggcaaggagcagaatgataacaccagcaatggcaccaacgactacataggcagtgtagagaaatggcgttaaatggctcaaaaaggcctgtacatacttctcccaaagcgccactgaaaagatggcatagcttaaaagatgaaagtgtccaaacacatcctgcttccttcattggggaagttttaaaaaaaagtttagatgttgcctttacagttgcctttcaattcagtgttatactgtgtgtaggtaaaacaaatctcaatatggaattaaattgtctttttggggttggactaaatatgaaatccgaaagccaaaccagactcaccagaaattgctgtttagatattttaagaagttcttaaattagttatggagacaaagtgaaaacataaaatgtgaccatttaacttatggctaagaaatggactttaaattattcatgatacactgttaaaacccaatcttggaatcaaatattttttccaggggtgagaataagtataaacataaagcaactaaaatgaaacataaaaccttttattttcttctgattttaacaaggaatctatttaaatagaataacaactgatggtgaatcttaccgagctgtagaaaataaaaaattcctctccaaacatgggtagttttatgtcaaaatattggcttttcaagaacaggactcatatcttgatatttaagagatgtttaaaattttaaactttttctaccttctactgtttaaaggttttacacagggtgtatctcacattaaacaaaacacctttttttcaaaatgaaataccaatgtaaagatctaatttccaggcgctttcagggcactgtaatttcaacaatactggaatcattttggcgctgcttctcattcattttaaggcttctctgaattgtgctcattccaaattaacccatgtatagaatctttcttcatcatctaaatggtgtgttgctgaagttattgtggtatataatcctggattaaagtcaggacttt

SEQ ID NO: 31 - DNA, 2080 bp, Homo sapiens
  misc_feature: Incyte ID No 3538709CB1
tgtttcggcggccgcgggatgcccctgcgctgaccgccaggggcaggtgcccgcccgcgtagacgcacccggcctgaccccgcgccaccatgtaaacagcgccagcaggcggacgctggcttctccgcctgggacccctccgccccgacccgggccccgcggccctcgatgaggacacaccatgctgaccggggtgaccgacggtatcttctgttgcctgctgggcacgccccccaacgccgtggggccactggagagcgtcgagtccagcgatggctacacctttgtagaggtcaagcccggccgcgtgctgcgggtgaagcatgcaggacccgccccagccgctgccccacctccaccatcatccgcatcctcggatgcagcccagggggacctctccggcttggtccgctgtcagcgccggatcaccgtgtaccgcaatgggcggttgctggtggaaaacctgggccgagcccctcgagccgacctcctacacgggcagaatggctctggggagccgccggccgccctggaggtggagctggcagatccggcgggcagcgatggccgcttggcccccggcagcgcaggcagcggcagcggcagtggcagtggtgggcggcggcggcgagccaggcgccccaagaggaccatccatattgactgtgagaagcgcatcactagctgcaaaggcgcccaggccgacgtggtgctctttttcatccatggtgtcggcggttccctggccatctggaaggagcagctggacttctttgtgcgcctaggctatgaggtggtggctcctgacctggccggccacggggccagctctgcgccccaggtggccgcagcctacaccttctatgcgctggctgaggacatgcgagcaatcttcaagcgctatgccaagaagcgaaatgtgctcattggccattcctacggtgtctctttctgcacattcctggcacatgagtacccagacctagtgcacaaggtgatcatgatcaatggcgggggccctacggcgctggagcccagcttctgctcaatcttcaacatgcccacctgcgtcctgcactgcttgtcgccctgcctggcctggagcttcctcaaggccggcttcgcccgccaaggagccaaggagaagcagctgttaaaggagggcaacgctttcaacgtgtcatccttcgtactccgggccatgatgagcggccagtactggcccgagggcgacgaggtctaccacgccgagctcaccgtgcccgtcctgcttgtccacggcatgcacgataagtttgtgccggtggaggaagaccagcgcatggccgagatcctgctcctggcattcctgaagctcatcgacgagggcagccacatggtgatgctggaatgccctgagacggtcaacacgctgctccacgaattcctgctctgggagcccgagccctcgcccaaggctctaccggagccactgccggcgcctccagaagacaagaagtagccgctgggccggcggggcatcgcttggtgagcacagccgcagcaggaggaggcccgagcctgcgccaggtctgcagcgcagaccacctgggcgggccgttcgctccggtgggcggggccaggtcagggagacgcccccaggctgcctgggcggggcgtggcatccgagggagcccagcggacattccgctctccgcttccgtcccgcggggcccatcggcgttttggggccgcagccgggaccctcacggaagatgaccttgtacagaagctctccctcaccttccccccaacgccacggccaaggcaggccccccaccccgctgtcttccgtgtcagccgtgcttgatcctgggacccacgagccccacagggaccctcgaggccccatcccgttatccgagacccttcctaccccccattcctcggcgctgggagctatttttgcccaagggggggggatgggggggctggcgccaccgaacctgcacatctcaacttgtaactcaataaacagaagtgacaatcggaaaaaaaaaaaaaaaaaaaaaaaaaaaaaaaaaaaaaaagaaaaaaaaaa

SEQ ID NO: 32 - DNA, 2219 bp, Homo sapiens
  misc_feature: Incyte ID No 71563101CB1
ggcctccaatctgcttccatgggggttggctttctgagtgggagaaatgactctaatctggagacatttgctgagacccttgtgcctggtcacttccgctcccaggatccttgagatgcatcctttcctgagcctaggtacttcccggacatcagtaaccaagctcagtcttcatacaaagcccagaatgcctccatgtgacttcatgcctgaaagataccagtcccttggctacaaccgtgtcctggaaatccacaaggaacatctttctcctgtggtgacggcatatttccagaaacccctgctgctccaccaggggcacatggagtggctctttgatgctgaaggaaacagatacctggatttcttttccgggattgttactgtcagtgttggccactgccacccaaaggtgaatgcagtggcacaaaagcagctcggccgcctgtggcatacaagcaccgtcttcttccaccctccaatgcatgaatatgcagagaagcttgccgcacttcttcctgagcctcttaaggtcattttcttggtgaacagtggctcagaagccaatgagctggccatgctgatggccagggcgcactcaaacaacatagacatcatttctttcagaggagcctaccatggatgcagtccttacacacttggcttgacaaacgtagggatctacaagatggaactccctggtgggacaggttgccaaccaacaatgtgtccagatgtttttcgtggcccttggggaggaagccactgtcgagattctccagtgcaaacaatcaggaagtgcagctgtgcaccagactgctgccaagctaaagatcagtatattgagcaattcaaagatacgctgagcacatctgtggccaagtcaattgctggatttttcgcagaacctattcaaggtgtgaatggagttgtccagtacccaaaggggtttctaaaggaagcctttgagctggtgcgagcaaggggaggcgtgtgcattgcagatgaagtgcagacaggatttggaaggttgggctctcacttctggggcttccaaacccacgatgtcctgcctgacattgtcaccatggctaaagggattgggaatggctttcccatggcagcagtcataaccactccagagattgccaaatctttggcgaaatgcctgcagcacttcaacacctttggagggaaccccatggcctgtgccattggatctgctgtgcttgaggtgattaaagaagaaaatctacaggaaaacagtcaagaagttgggacctacatgttactaaagtttgctaagctgcgggatgaatttgaaattgttggagacgtccgaggcaaaggcctcatgataggcatagaaatggtgcaggataagataagctgtcggcctcttccccgtgaagaagtaaatcagatccatgaggactgcaagcacatgggactcctcgttggcagaggcagcattttttctcagacatttcgcattgcgccctcaatgtgcatcactaaaccagaagttgattttgcagtagaagtatttcgttctgccttaacccaacacatggaaagaagagctaagtaacattgtcagaaataaataaaaccacaagtctcaagaatttgccacgtatgttcaagggtgaatttgaagaatttcagaaccactggtatccagagaaagcctgcagctctccacaggagctgtaaaagtcatggttgactgcctaccaaccatatttgttagcagagcccctcttatcttgagaactccattcttcagggaaaggatctccctagctcagagaataaatcctaattagtttatgttaggtatggtaatttgattcccctttgcagtgattggtttatgcatgaatatgtgatgtatttttgtccagtgaatcttgaagaaaaatcttttggtggaggtgccttcagggaaagttttcttcaccctcactcttcagttcaagaagagatgtcttcttgttgcgctgagaacaccatatgttcatgacgagattcctggcaccatgtcagccggcttgtagtcatgaggacaaccctttttggtgaggttggaagatggatggaagccaagtgcttagtgatgtcaaagaagcactcacttaagcattcctggagccaccctacctcagggcctcttgatatttgaggtaataaaattcattgttctgtataaaaaaaaa

SEQ ID NO: 33 - DNA, 681 bp, Homo sapiens
  misc_feature: Incyte ID No 7472027CB1
atgagactatgtgaaaagaccgaactacagttgattggtgtacctgaaagtgacagggagaatggaaccaagttggaaaacacatttcaggatattatccaggagaacttccccaacctagcaagacaggccaacattcaaattcagatggctggtggatctatctggattgaagggattccttttcccagcaataattttacagacctgagacgtttgcaagatgaaattgtgttgcgggatgaagatgtcattacactttcttacccaaagtcaggaagcttttggatagtggaaatcatcagtctgatccactccaagggagatcctagttgggtccaatctgttgttccctgggatcgttcaccatggatagaagttaaacgtaagaaagcaggtttagagagtcagaagggcccacacctctacacctcccaccttcccattcagctcttccccaagtcattcttgaattccaaggccaagtgtatttatcctcatgttctcatgcttgtggttctcatcctaggacataagagccagtggagtattgctataaagatatctgaaaatgcagaagcaacttcaaaactgggtaatgggcaaagattggaagagtttggagggctcagaagaagacaggaagatgaaagaagtttggaatttcttagagactgttaa

SEQ ID NO: 34 - DNA, 399 bp, Homo sapiens
  misc_feature: Incyte ID No 7480358CB1
agcccagaccagagtgtggcccctggagtcataatgatgaaggtcatgtacatgttgaagggccagagcccggtgcagggcaccatccactttgagcagaaggaaaatgaaccatttatggtgtcagaatgcattacaggattgactgaacgccagcacagattccatgttcatcagtttggagataatacaccaggctgtaccagggcagttccttactttaatcctttaaccaaaaaccacagtgggccaaggatcaagagaggcaggttggagacctgggtaatgtggccgctggcaaagatgtgtcgccacatgtctgttgaagattctctggtctcactctcaggacactattccatcactgcccacacaatggtgtccatgacaaccagatga

SEQ ID NO: 35 - DNA, 2302 bp, Homo sapiens
  misc_feature: Incyte ID No 1618256CB1
taactaatcggaaggcgctgtgaatttcacgtttctcgcgtagctgtaacgcgcggtatagttcttactcaagtcgtgccctgccagaaaggggaagatgcagcgagggagagacctagaaccgcgatcaaagctgcagcagctgctggggctggcggcacaaagggaggagggaggagcctgggcgctgagaaagttcttggggaaagttgagctgagccaagagtcgggcggtggctgggatgggcgggagggcccggcccgatttcccttgctgctccccttgtggcctgacgctgacagaggcaaaaatctgctaactcagggggcagactcaaccaagactgtgagcaggcctggggaatgaccccccgatctccaaccagtgccttccgcagctgcacggctgtctccagctgtctctgcccctcttcctggccctggctccatctctctgtcacctcacccttccctgtgccacatgggccctctctctcctgccaggacgctgcggctctggggacctcggagcctgggggtggctctgggagtcttcatgaccattggctttgcactccagctcttgggagggcccttccagaggaggctacctgggctacagctccgacagccctcggccccatccctacgaccagcccttccgtcctgcccaccccggcagcgactggtgttcctgaagacacataaatccgggagcagctctgtgctgagcctgcttcaccgctatggggaccagcacgggctgcgcttcgccctccctgcccgctaccagtttggctacccaaagctcttccaggcctctagggtaaaaggctaccgcccacagggtggaggcacccagctccccttccacatcctctgtcaccacatgaggttcaacctgaaagaggtacttcaggtcatgccttctgacagcttctttttttccattgtccgagacccagcggctctggctcgctctgccttctcctactataaatccacctcatcagccttccgcaagtcaccatctttggctgccttcctggccaatcctcgaggcttctacaggcctggggcccgtggggaccactacgctcgcaacttactatggtttgactttggcctgccctttcccccagagaagagggccaagagagggaatattcatccccccagagaccccaaccccccacagctgcaggtcttgccttctggtgctggccctcgagcccaaaccctcaatcccaatgccctcatccatcctgtttccactgttactgatcatcgcagccagatatcaagccctgcctctttcgatttggggtcttcatccttcatccagtggggtctggcctggctggactctgtctttgacctggtcatggtggctgagtacttcgatgagtcattggttctgctggcagatgccctgtgctggggtctagatgacgtggtgggcttcatgcacaatgcccaggctggacataagcagggcctcagcactgtcagcaacagtggactgactgcggaggaccggcagctgactgcacgggcccgagcctggaacaacctggactgggctctctatgtccacttcaaccgcagtctctgggcacggatagagaaatacggccagggccggctgcagacagctgtggccgagctccgggctcgccgagaggccctagcgaaacattgtctggtagggggtgaggcttctgaccccaaatacatcactgatcgccggttccgccccttccagtttgggtcagctaaggttttgggctatatacttcggagtggattgagcccccaagaccaagaggaatgtgagcgcctagctacccctgagctccagtacaaggacaagctggatgtcaagcagttcccccctaccgtctcactgcccctcaagacttcaaggccactctccccataaacatcagactacagatttaggtggaagagcagccatgtttgaagggcacatgtgatgagtggggggcagcaagatgccatttctgcatctcccagaagggatgagtctttgtcccgatgcaagccccctcttcgctgggctcccagcagtgcttccctcctccaccctccactcattttgttctttccccccaacttttttttttttgaaacggagtcttgctctgtcccccaggctggagtgcagtggcatgatctcggctcactgcaacctctgcctcccaggttcaagcgattctcctgcctcagcctccagagtagctaggattacagatacgtgccaccatacccggctaatttt

SEQ ID NO: 36 - DNA, 3341 bp, Homo sapiens
  misc_feature: Incyte ID No 3387823CB1
gaaacgaagagaaccctctgtccctggcagaatctgcatgtacatttcttgtctgtccttgtctctcttcttcctgtctggcccattgcagagagtattggaagtttccaaccattggtggtactctatgctcatcctacctcctttgctgaaagacagtgtggcagcgcccctgctgtctgcctactaccctgactgtgttggcatgagcccctcctgcaccagcacaaaccgcgccgctgccactggcaatgccagccctgggaagctggagcactccaaggctgccctctccgtgcacgttccaggcatgaaccgatacttccagcctttctaccagcccaatgagtgtggcaaagccctctgtgtgaggccggatgtgatggaactggatgagctctatgagtttccagagtattcccgagaccccaccatgtacctggctttgagaaacctcatcctcgcactgtggtatactaactgcaaagaagctcttactcctcagaaatgtattcctcacatcatcgtccggggtctcgtgcgtattcgatgcgttcaggaagtggagagaatactgtattttatgaccagaaaaggtctcatcaacactggagttctcagcgtgggagccgaccagtatcttctccctaaggactaccacaataaatcagtcatcattatcggggctggtccagcaggattagcagctgctaggcaactgcataactttggaattaaggtgactgtcctggaagccaaagacagaattggaggccgagtctgggatgataaatcttttaaaggcgtcacagtgggaagaggagctcagattgtcaatgggtgtattaacaacccagtagcattaatgtgtgaacaacttggcatcagcatgcataaatttggagaaagatgtgacttaattcaggaaggtggaagaataactgaccccactattgacaagcgcatggattttcattttaatgctctcttggatgttgtctctgagtggagaaaggataagactcagctccaagatgtccctttaggagaaaagatagaagaaatctacaaggcatttattaaggaatctggtatccaattcagtgagctggagggacaggtgcttcagttccatctcagtaacctggagtacgcctgtggcagcaaccttcaccaggtatctgctcgctcgtgggaccacaatgaattctttgcccagtttgctggtgaccacactctgctaactcccgggtactcggtgataattgaaaaactggcagaagggcttgacattcaactcaaatctccagtgcagtgtattgattattctggagatgaagtgcaggttaccactacagatggcacagggtattctgcacaaaaggtattagtcactgtaccactggctttactacagaaaggtgccattcagtttaatccaccgttgtcagagaagaagatgaaggctatcaacagcttaggcgcaggcatcattgaaaagattgccttgcaatttccgtatagattttgggacagtaaagtacaaggggctgacttttttggtcacgttcctcccagtgccagcaagcgagggctttttgccgtgttctatgacatggatccccagaagaagcacagcgtgctgatgtctgtgattgccggggaggctgtcgcatccgtgaggaccctggacgacaaacaggtgctgcagcagtgcatggccacgctccgggagctgttcaaggagcaggaggtcccagatcccacaaagtattttgtcactcggtggagcacagacccatggatccagatggcatacagttttgtgaagacaggtggaagtggggaggcctacgatatcattgctgaagacattcaaggaaccgtctttttcgctggtgaggcaacaaacaggcatttcccacaaactgttacaggggcatatttgagtggcgttcgagaagcaagcaagattgcagcattttaagaattcggtggacccagctttcttctgtaccccagatggggaaatttgaatcacatgttaaacctcagttttataagagggggaaaaaaccgtctctacatagtaaaactgaaatgtttctaaggcgatatgataatgcaaacctatttcatcactctaaaagcactgacctcaaaaaaccttataagcacttagatttaattgcattttccataggttcaactactgctgaaagtctggatttcagaataaagcagaatgtaagtttcagttgaggccatggatttgattgttccatggctggaagttccctttagatttcacattttatatggctgatcaattttcatacattgagaaaccaagtcaatcaagcaggaatcatttaaaaaccagataaagccatgtttttcttctgtgacaatttatcagtatctttaccaatgagccttaatttttatataggtccaatattgagcttttacttaaaatttagatagaacttttttttggatacagcacaaactccagttgacagtaaaatgaagcttctaggtattttgtattgtacatatttcctcctactgggtgttcaaaagaaatttaaattcaagtaccttttgtgataaaatgttttagatttgtgcacccattggcaaaacaggaaagtttccagataggtattgtatcattgagaatgcagcacagatagtgtgggcttcacactatagacacagaatatagctttttcttaaagccaaatttgggtgataggacactttaaatatccttaattttggcaaccactagcaaaaaaaacttgtcagaataatttaaccaagcccctctccacttcttttatttaaaagcactgattcaattgctaggaatatttttgcagatttttctttacagtattccataggcaggtccactggaaaactgcagaaaaatgtgagctctcctggtaaatagtatacattttataagctatattttaaaggcctaagaacatggcgagtatttacttttatcttttttttaaaaacactcatgacagaaaacagtctaataatatctcattctaaaataaaacactggttgcagggtcttcaggatgcctattttgccagaaacttcagtatacaggttagaaatatgcttttgtttttgaacataatatactggtttgctttaaagaagggactaaatatgactttaaagagacttcaaatattgagtattttaaaaatttaaaagtaggtcagtttataacgagtaaatacctaacacccaagaatgtgcagtgaacctcaggcggggatccttagttctaacggccgccccgtaggcg

SEQ ID NO: 37 - DNA, 777 bp, Homo sapiens
  misc_feature: Incyte ID No 55142051CB1
atgtcccctgccattgcattggccttcctgccactggtggtaacattgctggtgcggtaccggcactacttccgattgctggtgcgcacggtcttgctgcgaagcctccgagactgcctgtcagggctgcggatcgaggagcgggccttcagctacgtgctcacccatgccctgcccggtgaccctggtcacatcctcaccaccctggaccactggagcagccgctgcgagtacttgagccacatggggcctgtcaaaggtcagatcctgatgcggctggtggaggagaaggcccctgcttgtgtgctggaattgggaacctactgtggatactctaccctgcttattgcccgagccctgccccctgggggtcgccttcttactgtggagcgggacccacgcacggcagcagtggctgaaaaactcatccgcctggccggctttgatgagcacatggtggagctcatcgtgggcagctcagaggacgtgatcccgtgcctacgcacccagtatcagctgagtcgggcagacctggtgctcctggcacaccggccacgatgttacctgagggacctgcagctgctggaggcccatgccctactgccagcaggtgccaccgtgctggctgaccatgtgctcttccctggtgcaccccgcttcttgcagtatgctaagagctgtggccgctaccgctgccgcctccaccacactggccttccagacttccctgccatcaaggatggaatagctcagctcacctatgctggaccaggctga

SEQ ID NO: 38 - DNA, 3600 bp, Homo sapiens
  misc_feature: Incyte ID No 7395274CB1
gagagcagagcaggacactggcgccgcgggtcaggcagctgcgtgcgcgtctcctccaggcagcaaggggaacccgaggccgccggcgcccggaccatgtcgtctccggggccgtcgcagccgccggccgaggacccgccctggcccgcgcgcctcctgcgtgcgcctctggggctgctgcggctggaccccagcgggggcgcgctgctgctatgcggcctcgtagcgctgctgggctggagctggctgcggaggcgccgggcgcggggcatcccgcccgggcccacgccctggcctctggtgggcaacttcggtcacgtgctgctgcctcccttcctccggcggcggagctggctgagcagcaggaccagggccgcagggattgatccctcggtcataggcccgcaggtgctcctggctcacctagcccgcgtgtacggcagcatcttcagcttctttatcggccactacctggtggtggtcctcagcgacttccacagcgtgcgcgaggcgctggtgcagcaggccgaggtcttcagcgaccgcccgcgggtgccgctcatctccatcgtgaccaaggagaagggggttgtgtttgcacattatggtcccgtctggagacaacaaaggaagttctctcattcaactcttcgtcattttgggttgggaaaacttagcttggagcccaagattattgaggagttcaaatatgtgaaagcagaaatgcaaaagcacggagaagaccccttctgccctttctccatcatcagcaatgccgtctctaacatcatttgctccttgtgctttggccagcgctttgattacactaatagtgagttcaagaaaatgcttggttttatgtcacgaggcctagaaatctgtctgaacagtcaagtcctcctggtcaacatatgcccttggctttattaccttccctttggaccatttaaggaattaagacaaattgaaaaggatataaccagtttccttaaaaaaatcatcaaagaccatcaagagtctctggatagagagaaccctcaggacttcatagacatgtaccttctccacatggaagaggagaggaaaaataatagtaacagcagttttgatgaagagtacttattttatatcattggggatctctttattgctgggactgataccacaactaactctttgctctggtgcctgctgtatatgtcgctgaaccccgatgtacaagaaaaggttcatgaagaaattgaaagagtcattggcgccaaccgagctccttccctcacagacaaggcccagatgccctacacagaagccaccatcatggaagtgcagaggctaactgtggtggtgccgcttgccattcctcatatgacctcagagaacacagtgctccaagggtataccattcctaaaggcacattgatcttacccaacctgtggtcagtacatagagacccagccatttgggagaaaccggaggatttctaccctaatcgatttctggatgaccaaggacaactaattaaaaaagaaacctttattccttttgggatagggaagcgggtgtgtatgggagaacaactggcaaagatggaattattcctaatgtttgtgagcctaatgcagagtttcgcatttgctttacctgaggattctaagaagcccctcctgactggaagatttggtctaactttagccccacatccatttaatataactatttcaaggagatgaagagcatctccaagaagagatggtaaaaagatatataaatacatatccttctaagcagattcttcctactgcaaaggacagtgaatccagcaactcagtggatccaagctgggctcagaggtcggaaggagggtagagcacactgggaggtttcatcttggaggattcctcagcaggatacttcagccattttagtaatgcaggtctgtgatttgggggatagaaaacaaagtacctatgaaacgggatatctggattttacttgcagtggcttccaccgatgggccaatcttctcatttcttagtgcctcagacatcccatatgtaaaatgagagtaataaaacttggcttctctctacctctcagcactaatgatggtcaaatgccttacatcttttctgatatctctaaaatgctgttaagttctggagaagaacttcaggagaagaagatctatcagctggcttttaaagacctatgacaacatgaaagtggtgttcagcctggaatgctttgtcagagatgggtgtggatttaggttatactgggggagaacttttctcagcacagattctatgccagcttctttgggcttgttctgtcactatctttttgtttatgattttagtttttactttttgtagatgtgggatgaagtggactctgtcgtgtatattgaggaaaaaagaaattataattttaaaaaatcccttgtaggattattatctaaatttatatgtctaacttctactacaactacaggaacagtgagccttgctacttctttagtagcttcttggcagaattcctttctactgagttatttgcaaagatgcagctctacctttttacttaaggcctgaatggtgagcatggggattttgatactgggactcatcaggaaaggattctgctttcaaactatactgaacattcctgtcctagcgtccctgccaccaggcccaatgcatctgatccttgaatatactctcaaagaattcactctctttttattaagagaactaaattgtttctaaatgtagatggtccctctggaaaagcagttttcagcaggggtggtaaccccttcagagggagtttggaaatgtgtgggtatgattcttggttatcataatgatgggggtgctactggccttctgctgccatgggaccaggatgctaaatgtcaaggtagtcctatacagtgaagaattgtcctgctcaagatgccaggatttcccccagtgagaacatgctctaaggaatgaccacccctttcttttattctcccacagtgctccatgtacagaagtaagcatagcagtcatatgagcaaccacattcctgaacctttcctcatgctggctctacacttaatcctttacttgtatgtttctgtaattcttacataaattctattaagagggtggcatactgtagtggatgaagctgaggcttatagtaggtaaggcacaaagttaaaaagtaacatcactgggtttcaaacctactggtctctgtgactaaagaacactttcagaaccacttcttgattctgccaccacttgatcccataacaggctaccccttggcctcatgctggagttgtgtgtgtctgtcttcatcccaggctgagctccttgaggtgaggatgttgtgctgtttgcctccctcacagtgccttggtcttagtggatgcccagttgtcttgtgaatgacttttaagaagtgtacttaagagaaaaatcctaccttatttgaataattacaagtcatgtttttgttgcttaaaggtgataaatcagtgtatattatttgttaatgtccattaaagccagtttttaaaaaaaaaaaaaaaaaaaaaaaaa

(19) United States
(12) Patent Application Publication
(10) Pub. No.: US 2004/0110259 A1
(43) Pub. Date: Jun. 10, 2004

(54) Drug metabolizing enzymes

(76) Inventors: Mariah R Baugh, San Leandro, CA (US); Christopher M Bruns, Mountain View, CA (US); Debopriya Das, Mountain View, CA (US); Angelo Delegeane, Milpitas, CA (US); Ding Li, Creve Coeur, MO (US); Vicki S Elliott, San Jose, CA (US); Ameena R Gandhi, San Francisco, CA (US); Jennifer A Griffin, Fremont, CA (US); April J A Hafalia, Santa Clara, CA (US); Farrah A Khan, Des Plaines, IL (US); Preeti G Lal, Santa Clara, CA (US); Sally Lee, San Jose, CA (US); Dyung Aina M Lu, San Jose, CA (US); Yan Lu, Mountain View, CA (US); Chandra S Arvizu, San Jose, CA (US); Jayalaxmi Ramkumar, Fremont, CA (US); Huijun Z Ring, Foster City, CA (US); Madhusudan M Sanjanwala, Los Altos, CA (US); Y Tom Tang, San Jose, CA (US); Kavitha Thangavelu, Sunnyvale, CA (US); Michael Thornton, Oakland, CA (US); Catherine M Tribouley, San Francisco, CA (US); Narinder K Chawla, Union City, CA (US); Bridget A Warren, Encinitas, CA (US); Junming Yang, San Jose, CA (US); Monique G Yao, Carmel, IN (US); Henry Yue, Sunnyvale, CA (US)

Correspondence Address: INCYTE CORPORATION, 3160 PORTER DRIVE, PALO ALTO, CA, 94304, US

(21) Appl. No.: 10/343,593

(22) PCT Filed: Aug. 3, 2001
(86) PCT No.: PCT/US01/24382

(51) Int. Cl. C12Q 1/68; A01K 67/00; C07H 21/04; C12N 9/00

(52) U.S. Cl. 435/183; 435/320.1; 435/325; 435/69.1; 435/252.3; 800/8; 536/23.2

(57) ABSTRACT

The invention provides human drug metabolizing enzymes (DME) and polynucleotides which identify and encode DME. The invention also provides expression vectors, host cells, antibodies, agonists, and antagonists. The invention also provides methods for diagnosing, treating, or preventing disorders associated with aberrant expression of DME.

### TECHNICAL FIELD

This invention relates to nucleic acid and amino acid sequences of drug metabolizing enzymes and to the use of these sequences in the diagnosis, treatment, and prevention of autoimmune/inflammatory, cell proliferative, developmental, endocrine, eye, metabolic, and gastrointestinal disorders, including liver disorders, and in the assessment of the effects of exogenous compounds on the expression of nucleic acid and amino acid sequences of drug metabolizing enzymes.

### BACKGROUND OF THE INVENTION

The metabolism of a drug and its movement through the body (pharmacokinetics) are important in determining its effects, toxicity, and interactions with other drugs. The three processes governing pharmacokinetics are the absorption of the drug, distribution to various tissues, and elimination of drug metabolites. These processes are intimately coupled to drug metabolism, since a variety of metabolic modifications alter most of the physicochemical and pharmacological properties of drugs, including solubility, binding to receptors, and excretion rates. The metabolic pathways which modify drugs also accept a variety of naturally occurring substrates such as steroids, fatty acids, prostaglandins, leukotrienes, and vitamins. The enzymes in these pathways are therefore important sites of biochemical and pharmacological interaction between natural compounds, drugs, carcinogens, mutagens, and xenobiotics.

It has long been appreciated that inherited differences in drug metabolism lead to drastically different levels of drug efficacy and toxicity among individuals. For drugs with narrow therapeutic indices, or drugs which require bioactivation (such as codeine), these polymorphisms can be critical. Moreover, promising new drugs are frequently eliminated in clinical trials based on toxicities which may only affect a segment of the patient group. Advances in pharmacogenomics research, of which drug metabolizing enzymes constitute an important part, are promising to expand the tools and information that can be brought to bear on questions of drug efficacy and toxicity (See Evans, W. E. and R. V. Relling (1999) Science 286:487-491).

Drug metabolic reactions are categorized as Phase I, which functionalize the drug molecule and prepare it for further metabolism, and Phase 11, which are conjugative. In general, Phase I reaction products are partially or fully inactive, and Phase II reaction products are the chief excreted species. However, Phase I reaction products are sometimes more active than the original administered drugs; this metabolic activation principle is exploited by prorngs (e.g. L-dopa). Additionally, some nontoxic compounds (e.g. aflatoxin, benzo[α]pyrene) are metabolized to toxic intermediates through these pathways Phase I reactions are usually rate-limiting in drug metabolism. Prior exposure to the compound, or other compounds, can induce the expression of Phase I enzymes however, and thereby increase substrate flux through the metabolic pathways. (See Klaassen, C. D., Amdur, M. O. and J. Doull (1996) Casarett and Doull's Toxicology: The Basic Science of Poisons, McGraw-Hill, New York, N.Y., pp. 113-186; B. G. Katzung (1995) Basic and Clinical Pharmacology, Appleton and Lange, Norwalk, Conn., pp. 48-59; G. G. Gibson and P. Skett (1994) Introduction to Drug Metabolism, Blackie Academic and Professional, London.)

Drug metabolizing enzymes (DMEs) have broad substrate specificities. This can be contrasted to the immune system, where a large and diverse population of antibodies are highly specific for their antigens. The ability of DMEs to metabolize a wide variety of molecules creates the potential for drug interactions at the level of metabolism For example, the induction of a DME by one compound may affect the metabolism of another compound by the enzyme.

DMEs have been classified according to the type of reaction they catalyze and the cofactors involved. The major classes of Phase I enzymes include, but are not limited to, cytochrome P450 and flavin-containing monooxygenase. Other enzyme classes involved in Phase I-type catalytic cycles and reactions include, but are not limited to, NADPH cytochrome P450 reductase (CPR), the microsomal cytochrome b5/NADH cytochrome b5 reductase system, the ferredoxin/ferredoxin reductase redox pair, aldolketo reductases, and alcohol dehydrogenases. The major classes of Phase II enzymes include, but are not limited to, UDP glucuronyltransferase, sulfotransferase, glutathione S-transferase, N-acyltransferase, and N-acetyl transferase.

Cytochrome P450 and P450 Catalytic Cycle-Associated Enzymes

Members of the cytochrome P450 superfamily of enzymes catalyze the oxidative metabolism of a variety of substrates, including natural compounds such as steroids, fatty acids, prostaglandins, leukotrienes, and vitamins, as well as drugs, carcinogens, mutagens, and xenobiotics. Cytochromes P450, also known as P450 heine-thiolate proteins, usually act as terminal oxidases in multi-component electron transfer chains, called P450-containing monooxygenase systems. Specific reactions catalyzed include hydroxylation, epoxidation, N-oxidation, sulfooxidation, N-, S-, and O-dealkylations, desulfation, deamination, and reduction of azo, nitro, and N-oxide groups. These reactions are involved in steroidogenesis of glucocorticoids, cortisols, estrogens, and androgens in animals; insecticide resistance in insects; herbicide resistance and flower coloring in plants; and environmental bioremediation by rnicroorganisms. Cytochrome P450 actions on drugs, carcinogens, mutagens, and xenobiotics can result in detoxification or in conversion of the substance to a more toxic product Cytocliromes P450 are abundant in the liver, but also occur in other tissues; the enzymes are located in microsomes. (See EXPASY ENZYME EC 1.14.14.1; PrositePDOC00081 CytochromeP450 cysteine heme-iron ligand signature; PRINTS EP4501 E-Class P450 Group I signature; Graham-Lorence, S. and Peterson, J. A. (1996) FASEB J. 10:206-214.)

Four hundred cytochromes P450 have been identified in diverse organisms including bacteria, fungi, plants, and animals (Graham-Lorence, suira). The B-class is found in prokaryotes and fungi, while the E-class is found in bacteria, plants, insects, vertebrates, and mammals. Five subclasses or groups are found within the larger family of E-class cytochromes P450 (PRINTS EP4501 E-Class P450 Group I signature).

All cytochromes P450 use a heme cofactor and share structural attributes. Most cytochromes P450 are 400 to 530 amino acids in length. The secondary structure of the enyyme is about 70% alpha-helical and about 22% beta-sheet. The region around the heme-binding site in the C-terminal part of the protein is conserved among cytochromes P450. A ten amino acid signature sequence in this heme-iron ligand region has been identified which includes a conserved cysteine involved in binding the heme iron in the fifthl coordination site. In eukaryotic cytochromes.P450, a membrane-spanning region is usually found in the first 15-20 amino acids of the protein, generally consistingof approximately 15 hydrophobic residues followed by a positively charged residue. (See Prosite PDOC00081, supra; Graham-Lorence, sunra.) Cytocrhrome P450 enzymes are involved in cell proliferation and development. The enzymes have rolesin chemical mutagenesis and carcinogenesis by metabolizing chemicals to reactive intermediates that form adducts with DNA (Nebert, D. W. and Gonzalez, F. J. (1987) Ann. Rev. Biochem. 56:945-993). These adducts can cause nucleotide changes and DNA rearrangements that lead to oncogenesis. Cytochrome P450 expression in liver and other tissues is induced by xenobiotics such as polycyclic aromatic hydrocarbons, peroxisomal proliferators, phenobarbital, and the glucocorticoid dexamethasone (Dogra, S. C. et al. (1998) Clin. Exp. Pharmacol. Physiol. 25:1-9). A cytochrome P450 protein may participate in eye development as mutations in the P450 gene CYPLB 1 cause primary congenital glaucoma (Online Mendelian Inheritance in Man (OMIM) *601771 Cytochrome P450, subfamily 1 (dioxin-inducible), polypeptide 1; CYP1B1).

Cytochromes P450 are associated with inflammation and infection. Hepatic cytochrome P450 activities are profoundly affected by various infections and inflammatory stimuli, some of which are suppressed and some induced (Morgan, E. T. (1997) Drug Metab. Rev. 29:1129-1188). Effects observed in vivo canbe mimicked by pro prifammatory cytokines and interferons. Autoantibodies to two cytochrome P450 proteins were found in patients with autoimmune polyenodocrinopathy-candidiasis-ectodermal dystrophy (APECED), a polyglandular autoimmune syndrome (OMIM *240300 Autoimmune polyenodocrinopathy-candidiasis-ectodermal dystrophy).

Mutations in cytochromes P450 have been linked to metabolic disorders, including congenital adrenal hyperplasia, the most common adrenal disorder of infancy and childhood; pseudovitarrn D-deficiency rickets; cerebrotendinous xanthomatosis, a lipid storage disease characterized by progressive neurologic dysfimction, premature atherosclerosis, and cataracts; and an inherited resistance to the anticoagulant drugs coumarin and warfarin (Isselbacher, K. J. et al. (1994) Harrison's Principles of Internal Medicine, McGraw-Hill, Inc. New York, N.Y., pp. 1968-1970; Takeyama, K. et al. (1997) Science 277:1827-1830; Kitanaka, S. et al. (1998) N. Engl. J. Med. 338:653-661; OMIM *213700 Cerebrotendinous xanthomatosis; and OMIM #122700 Coumarin resistance). Extremely high levels of expression of the cytochrome P450 protein aromatase were found in a fibrolamellar hepatocellular carcinoma from a boy with severe gynecomastia (feminization) (Agarwal, V. R. (1998) J. Clin. Endocrinol. Metab. 83:1797-1800).

The cytochrome P450 catalytic cycle is completed through reduction of cytochrome P450 by NADPH cytochrome P450 reductase (CPR). Another microsomal electron transport system consisting of cytochrome b5 and NADPH cytochrome b5 reductase has been widely viewed as a minor contnrbutor of electrons to the cytochrome P450 catalytic cycle. However, a recent report by Lamb, D. C. et al. (1999; FEBS Lett. 462:283-8) identifies a Candida albicans cytochrome P450 (CYP51) which canbe efficiently reduced and supported by the microsomal cytochrome b5/NADPH cytochrome b5 reductase system. Therefore, there are likely many cytochromes P450 which are supported by this alternative electron donor system.

Cytochrome bS reductase is also responsible for the reduction of oxidized hemoglobin (methemoglobin, or ferrihemoglobin, which is unable to carry oxygen) to the active hemoglobin (ferrohemoglobin) in red blood cells. Methemoglobinemia results when there is a high level of oxidant drugs or an abnormal hemoglobin (hemoglobin M) which is not efficiently reduced. Methemoglobinemia can also result from a hereditary deficiency in red cell cytochrome b5 reductase (Reviewed in Mansour, A. and Lurie, A. A. (1993) Am. J. Hematol. 42:7-12).

Members of the cytochrome P450 family are also closely associated with vitamin D synthesis and catabolism. Vitamin D exists as two biologically equivalent prohormones, ergocalciferol (vitamin D₂), produced in plant tissues, and cholecalciferol (vitamin D₃), produced in animal tissues. The latter form, cholecalciferol, is formed upon the exposure of 7-dehydrocholesterol to near ultraviolet light (i.e., 290-310 nm), normally resulting from even minimal periods of skin exposure to sunlight (reviewed in Miller, W. L. and Portale, A. A. (2000) Trends Endocrinol. Metab. 11:315-319).

Both prohormone forms are further metabolized in the liver to 25-hydroxyvitamin D (25(OH)D) by the enzyme 25-hydroxylase. 25(OH)D is the most abundant precursor form of vitamin D which must be further metabolized in the kidney to the active form, 1,α,25-dihydroxyvitamin D (1α,25(OH)₂D), by the enzyme 25-hydroxyvitamin D 1α-hydroxylase (1α-hydroxylase). Regulation of 1α,25(OH)₂D production is primarily at this final step in the synthetic pathway. The activity of 1α-hydroxylase depends upon several physiological factors including the circulating level of the enzyme product (1α,25(OH)₂D) and the levels of parathyroid hormone (PTH), calcitonin, insulin, calcium, phosphorus, growth hormone, and prolactin. FPrthermnore, extrarenal 1α-hydroxylase activity has been reported, suggesting that tissue-specific, local regulation of 1α, 25(OH)2D production may also be biologically important. The catalysis of 1α,25(OH)₂D to 24,25-dihydroxyvitam in D (24,25(OH)₂D), involving the enzyme 25-hydroxyvitamin D 24-hydroxylase (24-hydroxylase), also occurs in the kidney. 24-hydroxylase can also use 25(OH)D as a substrate (Shinki T. et al. (1997) Proc. Natl. Acad. Sci. U.S.A. 94:12920-12925; Miller, W. L. and Portale, A. A. supra; and references within).

Vitamin D 25-hydroxylase, la-hydroxylase, and 24-hydroxylase are all NADPH-dependent, type I (mitochondrial) cytochrome P450 enzymes that show a high degree of homology with other members of the family. Vitamin D 25-hydroxylase also shows a broad substrate specificity and may also perform 26-hydroxylation of bile acid intermediates and 25, 26, and 27-hydroxylation of cholesterol (Dilworth, F. J. et al (1995) J. Biol. Chem. 270:16766-16774; Miller, W. L. and Portale, A. A. supra; and references within).

The active form of vitamin D (1α,25(OH)₂D) is involved in calcium and phosphate homeostasis and promotes the differentiation of myeloid and skin cells. Vitamin D deficiency resulting from deficiencies in the enzymes involved in vitamin D metabolism (e.g., 1α-hydroxylase) causes hypocalcemia, hypophosphatemia, and vitamin D-dependent (sensitive) rickets, a disease characterized by loss of bone density and distinctive clinical features, including bandy or bow leggedness accompanied by a waddling gait. Deficiencies in vitamin D 25-hydroxylase cause cerebrotendinous xanthomatosis, a lipid-storage disease characterized by the deposition of cholesterol and cholestanol in the Achilles' tendons, brain, lungs, and many other tissues. The disease presents with progressive neurologic dysfunction, including postpubescent cerebellar ataxia, atherosclerosis, and cataracts. Vitamin D 25-hydroxylase deficiency does not result in rickets, suggesting the existence of alternative pathways for the synthesis of 25(OH)D (Griffin, J. E. and Zerwekh, J. E. (1983) J. Clin Invest 72:1190-1199; Gamblin, G. T. et al. (1985) J. Clin. Invest 75:954-960; and W. L. and Portale, A. A. supra).

Ferredoxin and ferredoxin reductase are electron transport accessory proteins which support at least one human cytochrome P450 species, cytochrome P450c27 encoded by the CYP27 gene (Dilworth, F. J. et al. (1996) Biochem. J. 320:267-71). A Strentomyces griseus cytochrome P450, CYP104D1, was heterologously expressed in E. coli and found to be reduced by the endogenous ferredoxin and ferredoxin reductase enzymes (Taylor, M. et al. (1999) Biochem. Biophys. Res. Commun. 263:838-42), suggesting that many cytochrome P450 species may be supported by the feffedoxin/ferredoxin reductase pair. Ferredoxin reductase has also been found in a model drug metabolism system to reduce actinomycin D, an antitumor antibiotic, to a reactive free radical species (Flitter, W. D. and Mason, R. P. (1988) Arch. Biochem. Biophys. 267:632-9).

Flavin-Containing Monooxygenase (FMO)

Flavin-containing monooxygenases oxidize the nucleophilic nitrogen, sulfur, and phosphorus heteroatom of an exceptional range of substrates. Like cytochromes P450, FMOs are microsomal and use NADPH and O₂; there is also a great deal of substrate overlap with cytochromes P450. The tissue distribution of FMOs includes liver, kidney, and lung.

There are five different known isoforms of FMO in mammals (FMO1, FMO2, FMO3, FMO4, and FMO5), which are expressed in a tissue-specific manner. The isoforms differ in their substrate specificities and other properties such as iniubition by various compounds and stereospecificity of reaction. FMOs have a 13 amino acid signature sequence, the components of which span the N-terminal two-thirds of the sequences and include the FAD binding region and the FATGY motif which has been found in many N-hydroxylating enzymes (Stehr, M. et al. (1998) Trends Biochem. Sci. 23:56-57; PRINTS FMOXYGENASE Flavin-containing monooxygenase signature).

Specific reactions include oxidation of nucleophilic tertiary amines to N-oxides, secondary amines to hydroxylamines and nitrones, primary amines to hydroxylamines and oximes, and suLfur-containing compounds and phosphines to S- and P-oxides. Hydrazines, iodides, selenides, and boron-contaning compounds are also substrates. Although FMOs appear similar to cytochromes P450 in their chemistry, they can generally be distinguished from cytochromes P450 in vitro based on, for example, the higher heat lability of FMOs and the nonionic detergent sensitivity of cytochromes P450; however, use of these properties in identification is complicated by further variation among FMO isoforms with respect to thermal stability and detergent sensitivity.

FMOs play important roles in the metabolism of several drugs and xenobiotics. FMO (FMO3 in liver) is predominantly responsible for metabolizing (S)-nicotine to (S)-nicotine N-1′-oxide, which is excreted in urine. FMO is also involved in S-oxygenation of cimetidine, an H₂-antagonist widely used for the treatment of gastric ulcers. Liverxpressed forms of FMO are not under the same regulatory control as cytochrome P450. In rats, for example, phenobarbital treatment leads to the induction of cytochrome P450, but the repression of FMO1.

Endogenous substrates of FMO include cysteamine, which is oxidized to the disulfide, cystamine, and trimethylamine IMA), which is metabolized to trimethylamine N-oxide. TMA smells like rotting fish, and mutations in the FMO3 isoform lead to large amounts of the malodorous free amine being excreted in sweat, urine, and breath. These symptoms have led to the designation fish-odor syndrome (OMIM 602079 Trimethylaminuria).

Lysyl Oxidase:

Lysyl oxidase (lysine 6-oxidase, LO) is a copper-dependent amine oxidase involved in the formation of connective tissue matrices by crosslinking collagen and elastin. LO is secreted as a N-glycosylated precuror protein of approximately 50 kDa Levels and cleaved to the mature form of the enzyme by a metalloprotease, although the precursor form is also active. The copper atom in LO is involved in the transport of electron to and from oxygen to facilitate the oxidative deamination of lysine residues in these extracellular matrix proteins. While the coordination of copper is essential to LO activity, insufficient dietary intake of copper does not influence the expression of the apoenzyme. However, the absence of the functional LO is linked to the skeletal and vascular tissue disorders that are associated with dietary copper deficiency. LO is also inhibited by a variety of semicarbazides, hydrazines, and amino nitrites, as well as heparin. Beta-aminopropionitrile is a commonly used inhibitor. LO activity is increased in response to ozone, cadmium, and elevated levels of hormones released in response to local tissue trauma, such as transforming growth factor-beta, platelet-derived growth factor, angiotensin IH, and fibroblast growth factor. Abnormalities in LO activity has been linked to Menkes syndrome and occipital horn syndrome. Cytosolic forms of the enzyme hae been implicated in abnormal cell proliferation (reviewed in Rucker, R. B. et al. (1998) Am. J. Clin. Nutr. 67:996S-1002S and Smith-Mungo. L. I. and Kagan, H. M. (1998) Matrix Biol 16:387-398).

Dihydrofolate Reductases

Dihydrofolate reductases (DEW) are ubiquitous enzymes that catalyze the NADPH-dependent reduction of dihydrofolate to tetrahydrofolate, an essential step in the de novo synthesis of glycine and purines as well as the conversion of deoxyuridine monophosphate (dULW) to deoxythymidine monophosphate (dTM). The basic reaction is as follows:

7,8Sdhydrofolate+NADPH→5,6,7,8-tetrahydrofolate+NADP⁺

The enzymes can be Iinhiited by a number of dihydrofolate analogs, including trimethroprim and methotrexate. Since an abundance of TMP is required for DNA synthesis, rapidly dividing cells require the activity of DHFR. The replication of DNA viruses (i.e., herpesvirus) also requires high levels of DHFR activity. As a result, drugs that target DHFR have been used for cancer chemotherapy and to inhibit DNA virus replication. (For similar reasons, thymidylate synthetases are also target enzymes.) Drugs that inlibit DHFR are preferentially cytotoxic for rapidly dividing cells (or DNA virus-infected cells) but have no specificity, resulting in the indiscriminate destruction of dividing cells. Furthermore, cancer cells may become resistant to drugs such as methotrexate as a result of acquired transport defects or the duplication of one or more DHFR genes (Stryer, L. (1988) Biochemistry. W. H Freeman and Co., Inc. New York. pp. 511-5619).

Aldo/Keto Reductases

Aldo/keto reductases are monomeric NADPH-dependent oxidoreductases with broad substrate specificities (Bohren, K. M. et al. (1989) J. Biol. Chem. 264:9547-51). These enzymes catalyze the reduction of carbonyl-containing compounds, including carbonyl-containing sugars and aromatic compounds, to the corresponding alcohols. Therefore, a variety of carbonyl-containing drugs and xenobiotics are likely metabolized by enzymes of this class.

One known reaction catalyzed by a family member, aldose reductase, is the reduction of glucose to sorbitol, which is then further metabolized to fructose by sorbitol dehydrogenase. Under normal conditions, the reduction of glucose to sorbitol is a minor pathway. In hyperglycemic states, however, the accumulation of sorbitol is implicated in the development of diabetic complications (OMIM *103880 Aldo-keto reductase family 1, member B1). Members of this enzyme family are also highly expressed in some liver cancers (Cao, D. et al. (1998) J. Biol. Chem. 273:11429-35).

Alcohol Dehydropenases

Alcohol dehydrogenases (ADIs) oxidize simple alcohols to the corresponding aldehydes. ADH is a cytosolic enzyme, prefers the cofactor NAD⁺, and also binds zinc ion. Liver contains the highest levels of ADH, with lower levels in kidney, lung, and the gastric mucosa Known ADH isoforms are dimeric proteins composed of 40 kDa subunits. There are five known gene loci which encode these subunits (a, b, g, p, c), and some of the loci have characterized allelic variants (b₁, b₂, b₃, g₁, g₂). The subunits can form homodimers and heterodimers; the subunit composition determines the specific properties of the active enzyme. The holoenzymes have therefore been categorized as Class I (subunit compositions aa, ab, ag, bg, gg), Class II (pp), and Class If[ (cc). Class I ADH isozymes oxidize ethanol and other small aliphatic alcohols, and are inhibited by pyrazole. Class II isozymes prefer longer chain aliphatic and aromatic alcohols, are unable to oxidize methanol, and are not inhibited by pyrazole. Class III isozymes prefer even longer chain aliphatic alcohols (five carbons and longer) and aromatic alcohols, and are not inhibited by pyrazole.

The short-chain alc hol dehydrogenases include a number of related enzymes with a variety of substrate specificities. Included in this group are the manialian enzymes D-beta-hydroxybutyrate dehydrogenase, (R)-3-hydroxybutyrate dehydrogenase, 15-hydroxvprostaglandin dehydrogenase, NADPH-dependent carbonyl reductase, corticosteroid 11-beta-dehydrogenase, and estradiol 17-beta-dehydrogenase, as well as the bacterial enzymes acetoacetyl-CoA reductase, glucose 1-dehydrogenase, 3-beta-hydroxysteroid dehydrogenase, 20-beta-hydroxysteroid dehydrogenase, ribitol dehydrogenase, 3-oxoacyl reductase, 2,3-dihydro-2,3-dihydroxybenzoate dehydrogenase, sorbitol-6-phosphate 2-dehydrogenase, 7-alpha-hydroxysteroid dehydrogenase, cis-1,2-dihydroxy-3,4-cyclohexadiene-1-carboxylate dehydrogenase, cis-toluene dihydrodiol dehydrogenase, cis-benzene glycol dehydrogenase, biphenyl-2,3-dihydro-2,3-diol dehydrogenase, N-acylmaninosamine 1-dehydrogenase, and 2-deoxy-D-gluconate 3-dehydrogenase (Krozowski, Z. (1994) J. Steroid Biochem. Mol. Biol. 51:125-130; Krozowski, Z. (1992) Mol. CellEndocrinol. 84:C25-3 1; and Marks, A.R. et al. (1992) J. Biol. Chem. 267:15459-15463).

UDP Glucuronyltransferase

Members of the UDP glucuronyltransferase family (UGTs) catalyze the transfer of a glucuronic acid group from the cofactor uridine diphosphate-glucuronic acid (UDP-glucuronic acid) to a substrate. The transfer is generally to a nucleophilic heteroatom (O, N, or S). Substrates include xenobiotics which have been functionalized by Phase I reactions, as well as endogenous compounds such as bilirubin, steroid hormones, and thyroid hormones. Products of glucuronidation are excreted in urine if the molecular weight of the substrate is less than about 250 g/mol whereas larger glucuronidated substrates are excreted in bile.

UGTs are located in the microsomes of liver, kidney, intestine, skin, brain, spleen, and nasal mucosa, where they are on the same side of the endoplasmic reticulum membrane as cytochrome P450 enzymes and flavin-containing monooxygenases, and therefore are ideally located to access products of Phase I drug metabolism. UGTs have a C-terminal membrane-spanning domain which anchors them in the endoplasmic reticulum membrane, and a conserved signature domain of about 50 amino acid residues in their C terminal section (Prosite PDC0C0359 UDP-glycosyltransferase signatture).

UGTs involved in drug metabolism are encoded by two gene families, UGT1 and UGT2. Members of the UGFl family result from alternative splicing of a single gene locus, which has a variable substrate binding domain and conttant region involved in cofactor binding and membrane insertion. Members of the UGI2 faily are encoded by separate gene loci and are divided into two families, UGT2A and UGM2B. The 2A subfamily is expressed in olfactory epithelium, and the 2B subfamily is expressed in liver microsomes. Mutations in UGT genes are associated with byperbilirubinemia (OMIM #143500 Hyperbilirubinemia I); Crigler-Najiar syndrome, characterized by intensehyperbilrubinemia from birth (OMIM #218800 Crigler-Najjar syndrome); and a milder form of hyperbilirubinemia termed Gilbert's disease (OMIM *191740 UGT1).

Sulfotransferase

Sulfate conjugation occurs on many of the same substrates which undergo 0-glucuronidation to produce a highly water-soluble sulfuric acid ester. Sulfotransferases (ST) catalyze this reaction by transferring SO₃⁻ from the cofactor 3′-phosphoadenosine-5′-phosphosulfate (PAPS) to the substrate. ST substrates are predominantly phenols and aliphatic alcohols, but also include aromatic amines and aliphatic amines, which are conjugated to produce the corresponding sulfamates. The products of these reactions are excreted mainly in urine.

STs are found in a wide range of tissues, including liver, kidney, intestinal tract, lung, platelets, and brain. The enzymes are generally cytosolic, and multiple forms are often coxpressed. For example, there are more than a dozen forms of ST in rat liver cytosol. These biochemically characterized STs fall into five classes based on their substrate preference: arylsulfotransferase, alcohol suffotransferase, estrogen sulfotransferase, tyrosine ester sulfotransferase, and bile salt sulfotransferase.

ST enzyme activity varies greatly with sex and age in rats. The combined effects of developmental cues and sex-related hormones are thought to lead to these differences in ST expression profiles, as well as the profiles of other DMEs such as cytochromes P450. Notably, the high expression of STs in cats partially compensates for their low level of UDP glucuronyltransferase activity.

Several forms of ST have been purified from human liver cytosol and cloned. There are two phenol sulfotransferases with different thermal stabilities and substrate preferences. The thermostable enzyme catalyzes the sulfation of phenols such as para-nitrophenol, ninoxidil, and acetaminophen; the thermolabile enzyme prefers monoamine substrates such as dopamine, epinephrine, and levadopa. Other cloned STs include an estrogen sulfotransferase and an N-acetylglucosamine6-O-sulfotransferase. This last enzyme is illustrative of the other major role of STs in cellular biochemistry, the modification of carbohydrate structures that may be important in cellular differentiation and maturation of proteoglycans. Indeed, an inherited defect in a sulfotransferase has been implicated in macular corneal dystrophy, a disorder characterized by a failure to synthesize mature keratan sulfate proteoglycans (Nakazawa, K et al. (1984) J. Biol. Chem. 259:13751-7; OMIM *217800 Macular dystrophy, corneal).

Galactosyltransferases

Galactosyltransferases are a subset of glycosyltransferases that transfer galactose (Gal) to the terminal N-acetylglucosarnine (GlcNAc) oligosaccharide chains that are part of glycoproteins or glycolipids that are free in solution (Kolbinger, F. et al. (1998) J. Biol. Chem. 273:433-440; Arnado, M. et al. (1999) Biochim. Biophys. Acta 1473:35-53). Galactosyltransferases have been detected on the cell surface and as soluble extracellular proteins, in addition to being present in the Golgi. β1,3-galactosyltransferases form Type I carbohydrate chains with Gal (01-3)GlcNAc linkages. Known human and mouse β1,3-galactosyltransferases appear to have a short cytosolic domain, a single transmembrane domain, and a catalytic domain with eight conserved regions. (Kolbinger, F. surra and Hennet, T. et al. (1998) J. Biol. Chem. 273:58-65). In mouse UDP-galactose:β-N-acetylglucosamine β1,3-galactosyltransferase-I region 1 is located at amino acid residues 78-83, region 2 is located at amino acid residues 93-102, region 3 is located at amino acid residues 116-119, region 4 is located at amino acid residues 147-158, region 5 is located at amino acid residues 172-183, region 6 is located at amino acid residues 203-206, region 7 is located at amino acid residues 236-246, and region 8 is located at amino acid residues 264-275. A variant of a sequence found within mouse UDP-galactose:P-N-acetylglucosamine β1,3-galactosyltransferase-I region 8 is also found in bacterial galactosyltransferases, suggesting that this sequence defines a galactosyltransferase sequence motif (Hennet, T. supra). Recent work suggests that brainiac protein is a β1,3-galactosyltransferase. (Yuan, Y. et al (1997) Cell 88:9-11; and Hennet, T. supra).

UDP-Gal:GlcNAc-1,4-galactosyltransferase (-1,4-GalT) (Sato, T. et al., (1997) EMBO J. 16:1850-1857) catalyzes the formation of Type II carbohydrate chains with Gal (β1-4)GicNAc linkages. As is the case with the β1,3-galactosyltransferase, a soluble form of the enzyme is formed by cleavage of the membrane-bound form. Amino acids conserved among β1,4-galactosyltransferases include two cysteines linked through a disulfide-bonded and a putative UDP-galactose-binding site in the catalytic domain (Yadav, S. and Brew, K (1990) J. Biol Chem. 265:14163-14169; Yadav, S. P. and Brew, K. (1991) J. Biol. Chem. 266:698-703; and Shaper, N. L. et al. (1997) 3. Biol. Chem. 272:31389-31399). β1,4-galactosyltransferases have several specialized roles in addition to synthesizing carbohydrate chains on glycoproteins or glycolipids. In mammals a β1,4-gaiactosyltransferase, as part of a heterodimer with α-lactalbumin, functions in lactating marmmnary gland lactose production. A β1,4-galactosyltransferase on ihe surface of sperm functions as a receptor that specifically recognizes the egg. Cell surface β1,4-galactosyltransferases also function in cell adhesion, cellbasal lamina interaction, and normal and metastatic cell migration. (Shur, B. (1993) Curr. Opin. Cell Biol. 5:854-863; and Shaper, J. (1995) Adv. Exp. Med. Biol. 376:95-104).

Glutathione S-Transferase

The basic reaction catalyzed by glutathione S-transferases (GST) is the conjugation of an electrophile with reduced glutathione (GSH). GSTs are homodimeric or heterodimeric proteins localized mainly in the cytosol, but some level of activity is present in microsomes as well The major isozymes share common structural and catalytic properties; in humans they have been classified into four major classes, Alpha, Mu, Pi, and Theta. The two largest classes, Alpha and Mu, are identified by their respective protein isoelectric points; pI ˜7.5-9.0 (Alpha), and pI ˜6.6 (Mu). Each GST possesses a common binding site for GSH and a variable hydrophobic binding site. The hydrophobic binding site in each isozymne is specific for particular electrophilic substrates. Specific amino acid residues within GSTs have been identified as important for these binding sites and for catalytic activity. Residues Q67, T68, D101, E104, and R131 are important for the binding of GSH (Lee, H -C et al. (1995) J. Biol. Chem. 270: 99-109). Residues R13, R20, and R69 are important for the catalytic activity of GST (Stenberg G et al.(1991) Biochem. J. 274: 549-55).

In most cases, GSTs perform the beneficial finction of deactivation and detoxification of potentially mutagenic and carcinogenic chemicals. However, in some cases their action is detrimental and results in activation of chemicals with consequent mutagenic and carcinogenic effects. Some forms of rat and human GSTs are reliable preneoplastic markers that aid in the detection of carcinogenesis. Expression of human GSTs in bacterial strains, such as Salmonella tylhimurium used in the well-known Ames test for mutagenicity, has helped to establish the role of these enzymes in mutagenesis. Dihalomethanes, which produce liver tumors in mice, are believed to be activated by GST. This view is supported by the finding that dihalomethanes are more mutagenic in bacterial cells expressing human GST than in untransfected cells (Thier, R. et al.(1993) Proc. Natl. Acad. Sci. USA 90: 8567-80). The mutagenicity of ethylene dibromide and ethylene dichloride is increased in bacterial cells expressing the human Alpha GST, A1-1, while the mutagenicity of aflatoxin B1 is substantially reduced by enhancing the expression of GST (Simula, T. P. et al. (1993) Carcinogenesis 14:1371-6). Thus, control of GST activity maybe useful in the control of mutagenesis and carcinogenesis.

GST has been implicated in the acquired resistance of many cancers to drug treatment, the phenomenon known as multi-drug resistance (MDR). MDR occurs when a cancer patient is treated with a cytotoxic drug such as cyclophosphamide and subsequently becomes resistant to this drug and to a variety of other cytotoxic agents as well. Increased GST levels are associated with some of these drug resistant cancers, and it is believed that this increase occurs in response to the drug agent which is then deactivated by the GST catalyzed GSH conjugation reaction. The increas d GST levels then protect the cancer cells from other cytotoxic agents which bind to GST. Increased levels of AI-1 in tumors has been linked to drug resistance induced by cyclophosphamide treatment (Dirven IA. et al. (1994) Cancer Res. 54: 6215-20). Thus control of GST activity in cancerous tissues may be useful in treating MDR in cancer patients.

Gamma-Glutamyl Transpeptidase

Gamma-glutamyl transpeptidases are ubiquitously expressed enzymes that initiate extracellular glutathione (GSH) breakdown by cleaving gamma-glutamyl amide bonds. The breakdown of GSH provides cells with a regional cysteine pool for biosynthetic pathways. Gamma-glutamyl transpeptidases also contribute to cellular antioxidant defenses and expression is induced by oxidative steress. The cell surface-localiz glycoproteins.are expressed at high levels in cancer cells. Studies have suggested that the high level of gamma-glutamyl transpeptidases activity present on the surface of cancer cells could be exploited to activate precursor drugs, resulting in high local concentrations of anti-cancer therapeutic agents (Hanigan, M. H. (1998) Chem. Biol. Interact 111-112:333-42; Taniguchi, N. and Ikeda, Y. (1998) Adv. Enzymol. Relat. Areas Mol. Biol 72:239-78; Chikhi, N. et al. (1999) Comp. Biochem. Physiol. B. Biochem. Mol. Biol. 122:367-80).

Acyltransferase

N-acyltransferase enzymes catalyze the transfer of an amino acid conjugate to an activated carboxylic group. Endogenous compounds and xenobiotics are activated by acyl-CoA synthetases in the cytosol, microsomes, and mitochondria. The acyl-CoA intermediates are then conjugated with an amino acid (typically glycine, glutamine, or taurine, but also ornithine, arginine, histidine, serine, aspartic acid, and several dipeptides) by N-acyltransferases in the cytosol or mitochondria to form a metabolite with an amide bond. This reaction is complementary to O-glucuronidation, but amino acid conjugation does not produce the reactive and toxic metabolites which often result from glucuronidation.

One well-characterized enzyme of this class is the bile acid-CoA:amino acid N-acyltransferase (BAT) responsible for generating the bile acid conjugates which serve as detergents in the gastrointestinal tract (Falany, C. N. et al. (1994) J. Biol. Chefm 269:19375-9; Johnson, M. R. et al. (1991) J. Biol. Chem. 266:10227-33). BAT is also useful as a predictive indicator for prognosis of hepatocellular carcinoma patients after partial hepatectomy (Furutani, M. et al.(1996) Hepatology 24:1441-5).

Acetyltransferases

Acetyltransferases have been extensively studied for their role in histone acetylation. Histone acetylation results in the relaxing of the chromatin structure in eukaryotic cells, allowing transcription factors to gain access to promoter elements of the DNA templates in the affected region of the genome (or the genome in general). In contrast, histone deacetylation results in a reduction in transcription by closing the chromatin structure and limiting access of transcription factors. To this end, a common means of stimulating cell transcription is the use of chemical agents that bit the deacetylation of histones (e.g., sodium butyrate), resulting in a global (albeit artifactual) increase in gene expression. The modulation of gene expression by acetylation also results from the acetylation of other proteins, including but not limited to, p53, GATA-1, MyoD, ACTR, TTFIE, TInIF and the high mobility group proteins (HMG). In the case of p53, acetylation results in increased DNA binding, leading to the stimulation of transcription of genes regulated by p53. The prototypic histone acetylase (HAT) is Gcn5 from Saccharomyces cerevisiae. Gcn5 is a member of a family of acetylases that includes Tetrahymena p55, human Gcn5, and human p300/CBP. Thstone acetylation is reviewed in (Cheung, W. L. et al. (2000) Curr. Opin. Cell Biol. 12:326-333 and Berger, S. L (1999) Curr. Opin. Cell Biol. 11:336-341). Some acetyltransferase enzymes posses the alpha/beta hydrolase fold (Center of Applied Molecular Engineering lnst. of Chemistry and Biochemistry—University of Salzburg, http://predict.sanger.ac.ukfirbm-course97/Docslms/) common to several other major classes of enzymes, including but not limited to, acetylcholinesterases and carboxylesterases (Structural Classification of Proteins, http://scop.mrc-bnb.cam.ac.uklscop/index.html).

N-Acetyltransferase

Aromatic amines and hydrazine-containing compounds are subject to N-acetylation by the N-acetyltransferase enzymes of liver and other tissues. Some xenobiotics can be O-acetylated to some extent by the same enzymes. N-acetyltransferases are cytosolic enzymes which utilize the cofactor acetyl-coenzyme A (acetyl-CoA) to transfer the acetyl group in a two step process. In the first step, the acetyl group is transferred from acetyl-CoA to an active site cysteine residue; in the second step, the acetyl group is transferred to the substrate amino group and the enzyme is regenerated.

In contrast to most other DoE classes, there are a limited number of known N-acetyltransferases. In humans, there are two highly similar enzymes, NAT1 and NAT2; mice appear to have a third form of the enzyme, NAT3. The human forms of N-acetyltransferase have independent regulation (NAT1 is widely-expressed, whereas NAT2 is in liver and giut only) and overlapping substrate preferences. Both enzymes appear to accept most substrates to some extent, but NAT1 does prefer some substrates (para-aminobenzoic acid, para-aminosalicylic acid, sulfamethoxazole, and sulfanilamide), while NAT2 prefers others (isoniazid, hydralazine, procainamide, dapsone, aminoglutethimide, and sulfamethazine).

Clinical observations of patients taking the antituberculosis drug isoniazid in the 1950s led to the description of fast and slow acetylators of the compound. These phenotypes were shown subsequently to be due to mutations in the NAT2 gene which affected enzyme activity or stability. The slow isoniazid acetylator phenotype is very prevalent in Middle Eastem populations (approx. 70%), and is less prevalent in Caucasian (approx. 50%) and Asian (<25%) populations. More recently, functional polymorphism in NATI has been detected, with approximately 8% of the population tested showing a slow acetylator phenotype (Butcher, N. J. et al. (1998) Pharmacogenetics 8:67-72). Since NATI can activate some known aromatic amine carcinogens, polymorphism in the widely-expressed NATI enzyme may be important in determining cancer risk (OMIM *108345 N-acetyltransferase 1).

Aminotransferases

Aminotransferases comprise a family of pyridoxal 5′-phosphate (PLP) -dependent enzymes that catalyze transformations of amino acids. Aspartate aminotransferase (AspAT) is the most extensively studied PLP-containing enzyme. It catalyzes the reversible transamination of dicarboxylic L-amino acids, aspartate and glutamate, and the corresponding 2-oxo acids, oxalacetate and 2-oxoglutarate. Other members of the family included pyruvate aminotransferase, branched-chain amino acid aminotransferase, tyrosine aminotransferase, aromatic aminotransferase, alanine:glyoxylate aminotransferase (AGT), and kynurenine aminotransferase (Vacca, R. A. et al. (1997) J. Biol. Chem. 272:21932-21937).

Primary hyperoxaluria type-1 is an autosomal recessive disorder resulting in a deficiency in the liver-specific peroxisomal enzyme, alanine:glyoxylate aniintransferase-1. The phenotype of the disorder is a deficiency in glyoxylate metabolism. In the absence of AGT, glyoxylate is oxidized to oxalate rather than being transaminated to glycine. The result is the deposition of insoluble calcium oxalate in the kidneys and urinary tract, ultimately causing renal failure (Lumb, M. J. et al. (1999) J. Biol. Chem. 274:20587-20596).

Kynurenine aminotransferase catalyzes the irreversible transaiination of the L-tryptophan metabolite L-kynurenine to form kynurenic acid. The enzyme may also catalyzes the reversible transamination reaction between L-2-amnuoadipate and 2-oxoglutarate to produce 2-oxoadipate and L-glutamate. Kynurenic acid is a putative modulator of glutunatergic neurotrasmission, thus a deficiency in kynurenine aminotransferase may be associated with pleotrophic effects (Buchli, R. et al. (1995) J. Biol.Chem. 270:29330-29335).

Copper-Zinc Superoxide Dismutases

Copper-zinc superoxide dismutases are compact homodimeric metalloenzymes involved in cellular defenses against oxidative damage. The enzymes contain one atom of zinc and one atom of copper per subunit and catalyze the dismutation of superoxide anions into O₂ and H₂O₂. The rate of dismutation is difflusion-limited and consequently enhanced by the presence of favorable electrostatic interactions between the substrate and enzyme active site. Examples of this class of enzyme have been identified in the cytoplasm of all the eukaryotic cells as well as in the periplasm of several bacterial species. Copper-zinc superoxide dismutases are robust enzymes that are highly resistant to proteolytic digestion and denaturing by urea and SDS. In addition to the compact structure of the enzymes, the presence of the metal ions and intrasubunit disulfide bonds is believed to be responsible for enzyme stability. The enzymes undergo reversible denaturation at temperatures as high as 70° C. (Battistoni, A. et al. (1998) J. Biol. Chem. 273:5655-5661).

Overexpression of superoxide dismutase has been implicated in enhancing freezing tolerance of trausgenic Alfalfa as well as providing resistance to environmental toxins such as the diphenyl ether herbicide, acifluorfen (McKersie, B. D. et al. (1993) Plant Physiol. 103:1155-1163). In addtion, yeast cells become more resistant to freeze-thaw damage following exposure to hydrogen peroxide which causes the yeast cells to adapt to further peroxide stress by upregulating expression of superoxide dismutases. In this study, mutations to yeast superoxide dismutase genes had a more detrimental effect on freeze-thaw resistance than mutations which affected the regulation of glutathione metabolism, long suspected of being important in determing an organisms survival through the process of cryopreservation (Jong-In Park, J -I. et al. (1998) J. Biol. Chem. 273:22921-22928).

Expression of superoxide dismutase is also associated with Mycobacterium tuberculosis, the organism that causes tuberculosis. Superoxide dismutase is one of the ten major proteins secreted by M. tuberculosis and its expression is upregulated approximately 5-fold in response to oxidative stress. M. tuberculosis expresses almost two orders of magnitude more superoxide dismutase than the nonpathogenic mycobacterium M. smegmatis, and secretes a much higher proportion of the expressed enzyme. The result is the secretion of 350-fold more enzyme by M. tuberculosis than M. smegmatis, providing substantial resistance to oxidative stress (Harth, G. and Horwitz, M.A. (1999) J. Biol.Chem. 274:4281-4292).

The reduced expression of copper-zinc superoxide dismutases, as well as other enzymes with anti-oxidant capabilities, has been implicated in the early stages of cancer. The expression of copper-zinc superoxide dismutases has been shown to be lower in prostatic intraepithelial neoplasia and prostate carcinomas, compared to normal prostate tissue (Bostwick, D.G. (2000) Cancer 89:123-134).

Phosphodiesterases

Phosphodiesterases make up a class of enzymes which catalyze the hydrolysis of one of the two ester bonds in a phosphodiester compound. Phosphodiesterases are therefore crucial to a variety of cellular processes. Phosphodiesterases include DNA and RNA endonucleases and exonucleases, which are essential for cell growth and replication, and topoisomerases, which break and rejoin nucleic acid strands during topological rearrangement of DNA. A Tyr-DNA phosphodiesterase functions in DNA repair by hydrolyzing dead-end covalent intermediates formed between topoisomerase 1 and DNA (Pouliot, J. J. et al. (1999) Science 286:552-555; Yang, S. -W. (1996) Proc. Natl. Acad. Sci. USA 93:11534-11539).

Acid sphingomyelinase is a phosphodiesterase which hydrolyzes the membrane phospholipid sphingomyelin to produce ceramide and phosphorylcholine. Phosphorylcholine is used in the synthesis of phosphatidylcholine, which is involved in numerous intracellular signaling pathways, while ceramide is an essential precursor for the generation of gangliosides, membrane lipids found in high concentration in neural tissue. Defective acid sphingomyelnase leads to a build-up of sphingomyelin molecules in lysosomes, resulting in Niemann-Pick disease (Schuchman, E. H. and S. R. Miranda (1997) Genet. Test 1:13-19).

Glycerophosphoryl diester phosphodiesterase (also known as glycerophosphodiester phosphodiesterase) is a phosphodiesterase which hydrolyzes deacetylated phospholipid glycerophosphodiesters to produce sn-glycerol-3-phosphate and an alcohol. Glycerophosphocholine, glycerophosphoethanolamine, glycerophosphoglycerol, and glycerophosphoinositol are examples of substrates for glycerophosphoryl diester phosphodiesterases. A glycerophosphoryl diester phosphodiesterase from E. coli has broad specificity for glycerophosphodiester substrates (Larson, T. J. et al (1983) J. Biol. Chem. 248:5428-5432).

Cyclic nucleotide phosphodiesterases (PDEs) are crucial enzymes in the regulation of the cyclic nucleotides cAMP and cGMP. cAMP and cGMP function as intracellular second messengers to transduce a variety of extracellular signals including hormones, light, and neurotransmitters. PDEs degrade cyclic nucleotides to their corresponding monophosphates, thereby regulating the intracellular concentrations of cyclic nucleotides and their effects on signal transduction. Due to their roles as regulators of signal transduction, PDEs have been extensively studied as chemotherapeutic targets (Perry, M. J. and G. A. Higgs (1998) Curr. Opin. Chem. Biol. 2:472481; Torphy, J. T. (1998) Am. J. Resp. Crit. Care Med. 157:351-370).

Families of mammalian PDEs have been classified based on their substrate specificity and affinity, sensitivity to cofactors, and sensitivity to inhibitory agents (Beavo, J. A. (1995) Physiol.Rev. 75:725-748; Conti, M. et al. (1995) Endocrine Rev. 16:370-389). Several of these families contain distinct genes, many of which are expressed in different tissues as splice variants. Wirhin PDE *families, there are multiple isozymes and multiple splice variants of these isozymes (Conti, M. and S. -L. C. Jin (1999) Prog. Nucleic Acid Res. Mol. Biol. 63:1-38). The existence of multiple PDE families, isozymes, and splice variants is an indication of the variety and complexity of the regulatory pathways involving cyclic nucleotides (Houslay, M. D. and G. Milligan (1997) Trends Biochem. Sci. 22:217-224).

Type 1 PDEs (PDEls) are Ca²⁺/calmodulin-dependent and appear to be encoded by at least three different genes, each having at least two different splice variants (Kakkar, R. et al. (1999) Cell Mol. Life Sci. 55:1164-1186). PDEls have been found in the lung, heart, and brain. Some PDE1 isozymes are regulated in vitro by phosphorylation/dephosphorylation. Phosphorylation of these PDE1 isozymes decreases the affinity of the enzyme for calmodulin, decreases PDE activity, and increases steady state levels of cAMP (Kaldkar, supra). PDEls may provide useful therapeutic targets for disorders of the central nervous system, and the cardiovascular and immune systems due to the involvement of PDEls in both cyclic nucleotide and calcium signaling (Perry, M. J. and G. A. Higgs (1998) Cuff. Opin. Chem. Biol. 2:472-481).

PDE2s are cGMP-stimulated PDEs that have been found in the cerebellum, neocortex, heart, kidney, lung, pulmonary artery, and skeletal muscle (Sadhu, K. et al. (1999) J. Histochem. Cytochem. 47:895-906). PDE2s are thought to mediate the effects of cAMP on catecholamine secretion, participate in the regulation of aldosterone (Beavo, supra), and play a role in olfactory signal transduction (Juilfs, D. M. et al. (1997) Proc. Natl. Acad. Sci. USA 94:3388-3395).

PDE3s have high affinity for both cGMP and cAMP, and so these cyclic nucleotides act as competitive substrates for PDE3s. PDE3s play roles in stimulating myocardial contractility, inlibiting platelet aggregation, relaxing vascular and airway smooth muscle, inhibiting proliferation of T-lymphocytes and cultured vascular smooth muscle cells, and regulating catecholamine-induced release of free fatty acids from adipose tissue. The PDE3 family of phosphodiesterases are sensitive to specific inhibitors such as cilostamide, enoximone, and lixazinone. Isozymes of PDE3 can be regulated by cAMP-dependent protein kinase, or by insulin-dependent kinases (Degerman, E. et al. (1997) J. Biol. Chem. 272:6823-6826).

PDE4s are specific for cAMP; are localized to airway smooth muscle, the vascular endothelium, and all inflammatory cells; and can be activated by cAMT-dependent phosphorylation. Since elevation of cAMP levels can lead to suppression of inflammatory cell activation and to relaxation of bronchial smooth muscle, PDE4s have been studied extensively as possible targets for novel anti-inflammatory agents, with special emphasis placed on the discovery of asthma treatments. PDE4 inhibitors are currently undergoing clinical trials as treatments for asthma, chronic obstructive pulmonary disease, and atopic eczema. All four known isozymes of PDE4 are susceptible to the inhibitor rolipram, a compound which has been shown to improve behavioral memory in mice (Barad, M. et al.(1998) Proc. Natl.Acad. Sci. USA 95:15020-15025). PDE4 inhibitors have also been studied as possible therapeutic agents against acute lung injury, endotoxemia, rheumatoid arthritis, multiple sclerosis, and various neurological and gastrointestinal indications (Doherty, A. M. (1999) Curr. Opin. Chem. Biol 3:466-473).

PDE5 is highly selective for cGMP as a substrate (Turko, I.V. et a]. (1998) Biochemistry 37:4200-4205), and has two allosteric cGMP-specific binding sites (McAllister-Lucas, L. M. et al. (1995) J. Biol. Chem. 270:30671-30679). Binding of cGMP to these allosteric binding sites seems to be important for phosphorylation of PDE5 by cGMP-dependent protein kinase rather than for direct regulation of catalytic activity. High levels of PDE5 are found in vascular smooth muscle, platelets, lung, and kidney. The inhibitor zaprinast is effective against PDE5 and PDEls. Modification of zaprinast to provide specificity against PDE5 has resulted in sildenafil (VIAGRA; Pfizer, Inc., New York N.Y.), a treatment for male erectile dysfunction (Terrett, N. et al. (1996) Bioorg. Med. Chem. Lett. 6:1819-1824). Thhibitors of PDE5 are currently being studied as agents for cardiovascular therapy (Perry, M. J. and G. A. Higgs (1998) Curr. Opin. Chem. Biol. 2:472-481).

PDE6s, the photoreceptor cyclic nucleotide phosphodiesterases, are crucial components of the phototransduction cascade. In association with the G-protein transducin, PDE6s hydrolyze cGMP to regulate cGMP-gated cation channels in photoreceptor membranes. In addition to the cGMP-binding active site, PDE6s also have two high-affinity cGMP-binding sites which are thought to play a regulatory role in PDE6 function (Artemyev, N. O. et al. (1998) Methods 14:93-104). Defects in PDE6s have been associated with retinal disease. Retinal degeneration in the rd mouse (Yan, W. et al. (1998) Invest. Opthalmol. Vis. Sci. 39:2529-2536), autosomal recessive retinitis pigmentosa in humans (Danciger, M. et al. (1995) Genomics 30:1-7), and rod/cone dysplasia 1 in Irish Setter dogs (Suber, M. L. et al. (1993) Proc. And. Acad. Sci. USA 90:3968-3972) have been attnrbuted to mutations in the PDE6B gene.

The PDE7 family of PDBs consists of only one known member having multiple splice variants (Bloom, T. J. and J. A. Beavo (1996) Proc. Natl. Acad. Sci. USA 93:14188-14192). PDE7s are cAMP specific, but little else is known about their physiological function. Although mRNAs encoding PDE7s are found in skeletal muscle, heart, brain, lung, kidney, and pancreas, expression of PDE7 proteins is restricted to specific tissue types (Han, P. et al (1997) J. Biol.Cheni 272:16152-16157; Perry, M. J. and G. A. Iiggs (1998) Curr. Opin. Chem. Biol. 2:472-481). PDE7s are very closely related to the PDE4 family; however, PDE7s are not inhibited by roliprarn, a specific inhibitor of PDE4s (Beavo, supra).

PDE8s are cAMP specific, and are closely related to the PDE4 family. PDE8s are expressed in thyroid gland, testis, eye, liver, skeletal muscle, heart, kidney, ovary, and brain. The cAMP-hydrolyzing activity of PDE8s is not ihiibited by the PDE iniitors rolipram, vinpocetine, mihiinone, IBMX (3-isobutyl-1-methylxanthine), or zaprinast, but PDE8s are inhibited by dipyridamole (Fisher, D. A. et al. (1998) Biochem. Biophys. Res. Conunun. 246:570-577; Hayashi, M. et al.(1998) Biochem. Biophys. Res. Commun. 250:751-756; Soderling, S.H: et al. (1998) Proc. Natl Acad. Sci. USA 95:8991-8996).

PDE9s are cGMP specific and most closely resemble the PDE8 family of PDEs. PDE9s are expressed in kidney, liver, lung, brain, spleen, and small intestine. PDE9s are not inhibited by sildenafil (VIAGRA; Pfizer, Inc., New York N.Y.), rolipram, vinpocetine, dipyridamole, or IBMX (3-isobutyl-1-methylxanthine), but they are sensitive to the PDE5 inhibitor zaprinast (Fisher, D.A. et al.(1998) J. Biol. Chem. 273:15559-15564; Soderling, S. H. et al. (1998) J. Biol. Chem. 273:15553-15558).

PDElOs are dual-substrate PDEs, hydrolyzing both cAMP and cGMP. PDE1Os are expressed in brain, thyroid, and testis. (Soderling, S. H. et al. (1999) Proc. Natl. Acad. Sci. USA 96:7071-7076; Fujishige, K. et al. (1999) J. Biol. Chem. 274:18438-18445; Loughney, KI et al (1999) Gene 234:109-117).

PDEs are composed of a catalytic domain of about 270-300 amino acids, an N-terminal regulatory domain responsible for binding cofactors, and, in some cases, a hydrophilic C-terminal domain of unknown function (Conti, M. and S. -L. C. Jin (1999) Prog. Nucleic Acid Res. Mol. Biol. 63:1-38). A conserved, putative zinc-binding motif, HDXXHXGXXN, has been identified in the catalytic domain of all PDEs. N-terminal regulatory domains include non-catalytic cGMP-binding domains in PDE2s, PDESs, and PDE6s; cabmodulin-bnding domains in PDEis; and domains containing phosphorylation sites in PDE3s and PDE4s. In PDE5, the N-terminal cGMP-binding domain spans about 380 amino acid residues and comprises tandem repeats of the conserved sequence motif N(R/K)XnFX₃DE (McAister-Lucas, L. M. et al. (1993) J. Biol. Chem. 268:22863-22873). The NKXnD motif has been shown by mutagenesis to be important for cGMP binding (Turko, I. V. et al (1996) J. Biol. Chem. 271:22240-22244). PDE families display approximately 30% amino acid identity within the catalytic domain; however, isozymes within the same family typically display about 85-95% identity in this region (e.g. PDE4A vs PDE4B). Furthermore, within a family there is extensive similarity (>60%) outside the catalytic domain; while across families, there is little or no sequence similarity outside this domain.

Many of the constituent functions of immune and inflammatory responses are inhibited by agents that increase intracellular levels of cAMP (Verghese, M. W. et al. (1995) Mol. Pharmacol. 47:1164-1171). A variety of diseases have been attributed to increased PDE activity and associated with decreased levels of cyclic nucleotides. For example, a form of diabetes insipidus in mice has been associated with increased PDE4 activity, an increase in low-Kₘ cAMP PDE activity has been reported in leukocytes of atopic patients, and PDE3 has been associated with cardiac disease.

Many inhibitors of PDEs have been identified and have undergone clinical evaluation (Perry, M. J. and G. A. Higgs (1998) Curr. Opin. Chem. Biol. 2:472-481; Torphy, T. J. (1998) Am. J. Respir. Crit Care Med. 157:351-370). PDE3 inhibitors are being developed as antithrombotic agents, antihypertensive agents, and as cardiotonic agents useful in the treatment of congestive heart failure. Rolipram, a PDE4 inhibitor, has been used in the treatment of depression, and other iribitors of PDE4 are undergoing evaluation as anti-inflammatory agents. Rolipram has also been shown to inlubit lipopolysaccharide (LPS) induced TNF-a which has been shown to enhance HIV-1 replication in vitro. Therefore, rolipram may inhibit HIV-1 replication (Angel, J. B. et al. (1995) AIDS 9:1137-1144). Additionally, rolipram, based on its ability to suppress the production of cytokines such as INF-a and b and interferon g, has been shown to be effective in the treatment of encephalomyelitis. Rolipram may also be effective in treating tardive dyskinesia and was effective in treating multiple sclerosis in an experimental animal model (Sommer, N. et al. (1995) Nat. Med. 1:244-248; Sasaki, H. et al. (1995) Eur. J. Pharmacol. 282:71-76).

Theophyline is a nonspecific PDE inhibitor used in the treatment of bronchial asthma and other respiratory diseases. Theophylline is believed to act on airway smooth muscle function and in an anti-inflammatory or immunomodulatory capacity in the treatment of respiratory diseases (Banner, K. H. and C. P. Page (1995) Eur. Respir. J. 8:996-1000). Pentoxifylline is another nonspecific PDE inbibitor used in the treatment of intermittent claudication and diabetes-induced peripheral vascular disease. Pentoxifylline is also known to block TNF-a production and may inhibit mv-1 replication (Angel et al., ura).

PDEs have been reported to affect cellular proliferation of a variety of cell types (Conti et al. (1995) Endocrine Rev. 16:370-389) and have been implicated in various cancers. Growth of prostate carcinoma cell lines DU145 and LNCaP was inhibited by delivery of cAMP derivatives and PDE inhibitors (Bang, Y. J. et al. (1994) Proc. Natl. Acad. Sci. USA 91:5330-5334). These cells also showed a permanent conversion in phenotype from epithelial to neuronal morphology. It has also been suggested that PDE inlibitors have the potential to regulate mesangial cell proliferation (Matousovic, K et al. (1995) J. Clin. Invest 96:401-410) and lymphocyte proliferation (Joulain, C. et al (1995) J. Lipid Mediat Cell Signal. 11:63-79). A cancer treatment has been descnbed that involves intracellular delivery of PDEs to particular cellular compartments of tumors, resulting in cell death (Deonarain, M. P. and A. A. Epenetos (1994) Br. J. Cancer 70:786-794).

Phosphotriesterases

Phosphotriesterases (PTE, paraoxonases) are enzymes that hydrolyze toxic organophosphorus compounds and have been isolated from a variety of tissues. The enzymes appear to be lacking in birds and insects and abundant in mammals, explain the reduced tolerance of birds and insects to organophosphorus compound (Vilanova, E. and Sogorb, M. A. (1999) Crit Rev. Toxicol. 29:21-57). Phosphotriesterases play a central role in the detoxification of insecticides by mammals. Phosphotriesterase activity varies among individuals and is lower in infants than adults. Knockout mice are markedly more sensitive to the organophosphate-based toxins diazoxon and chlorpyrifos oxon (Furlong, C. E., et al. (2000) Neurotoxicology 21:91-100). PTEs have attracted interest as enzymes capable of the detoxification of organophosphate-containing chemical waste and warfare reagents (e.g., parathion), in addition to pesticides and insecticides. Some studies have also implicated phosphotriesterase in atherosclerosis and diseases involving lipoprotein metabolism.

Thioesterases

Two soluble thioesterases involved in fatty acid biosynthesis have been isolated from mammalian tissues, one which is active only toward long-chain fatty-acyl thioesters and one which is active toward thioesters with a wide range of fatty-acyl chain-lengths. These thioesterases catalyze the chain-terminating step in the de novo biosynthesis of fatty acids. Chain termination involves the hydrolysis of the thioester bond which links the fatty acyl chain to the 4′-phosphopantetheine prosthetic group of the acylcarrier protein (ACP) subunit of the fatty acid synthase (Smith, S. (1981a) Methods Enzymol. 71:181-188; Smith, S. (1981b) Methods Enzymol. 71:188-200).

E. coli contains two soluble thioesterases, thioesterase I which is active only toward long-chain acyl thioesters, and thioesterase II (TEII) which has a broad chain-length specificity (Naggert, J. et al. (1991) J. Biol Chem. 266:11044-11050). E. coli TEII does not exhibit sequence similarity with either of the two types of mammalian thioesterases which function as chain-terminating enzymes in de novo fatty acid biosynthesis. Unlike the mammalian thioesterases, E. coli TEII lacks the characteristic serine active site gly—X—ser—X—gly sequence motif and is not inactivated by the serine modifying agent diisopropyl fluorophosphate. However, modification of histidine 58 by iodoacetamide and diethylpyrocarbonate abolished TEII activity. Overexpression of TEII did not alter fatty acid content in E. coli, which suggests that it does not function as a chain-terminating enzyme in fatty acid biosynthesis (Naggert et al, supra). For that reason, Naggert et al. (supra) proposed that the physiological substrates for E. coli TEII may be coenzyme A (CoA)fatty acid esters instead of ACP-phosphopanthetheine-fatty acid esters.

Caiboxylesterases

Mammalian carboxylesterases constitute a multigene family expressed in a variety of tissues and cell types. Isozyrnes have significant sequence homology and are classified primarily on the basis of amino acid sequence. Acetylcholinesterase, butyrylcholinesterase, and carboxylesterase are grouped into the serine super family of esterases (B-esterases). Other carboxylesterases included thyroglobulin, thrombin, Factor IX, gliotactin, and plasminogen. Carboxylesterases catalyze the hydrolysis of ester- and amide- groups from molecules and are involved in detoxification of drugs, environmental toxins, and carcinogens. Substrates for carboxylesterases include short- and long-chain acyl-glycerols, acylcarnitine, carbonates, dipiveftin hydrochloride, cocaine, salicylates, capsaicin, pahnitoyl-coenzyme A, imidapril, haloperidol, pyrrolizidine alkaloids, steroids, p-nitrophenyl acetate, malathion, butanilicaine, and isocarboxazide. The enzymes often demonstrate low substrate specificity. Carboxylesterases are also important for the conversion of prodrugs to their respective free acids, which may be the active form of the drug (e.g., lovastatin, used to lower blood cholesterol) (reviewed in Satoh, T. and Hosokawa, M. (1998) Annu. Rev. Pharmacol. Toxicol.38:257-288).

Neuroligins are a class of molecules that (i) have N-terminal signal sequences, (ii) resemble cell-surface receptors, (iii) contain carboxylesterase domains, (iv) are highly expressed in the brain, and (v) bind to neurexins in a calcium-dependent manner. Despite the homology to carboxylesterases, neuroligins lack the active site serine residue, implying a role in substrate binding rather than catalysis (Ichtchenko, K. et al. (1996) J. Biol. Chem. 271:2676-2682).

Squalene Epoxidase

Squalene epoxidase (squalene monooxygenase, SE) is a mricrosomal membrane-bound, FAD-dependent oxidoreductase that catalyzes the first oxygenation step in the sterol biosynthetic pathway of eukaryotic cells. Cholesterol is an essential structural component of cytoplasmic membranes acquired via the IDl. receptor-mediated pathway or the biosynthetic pathway. In the latter case, all 27 carbon atoms in the cholesterol molecule are derived from acetyl-CoA (Stryer, L., supra). SE converts squalene to 2,3(S)-oxidosqualene, which is then converted to lanosterol and then cholesterol. The steps involved in cholesterol biosynthesis are summarized below (Stryer, l. (1988) Biochemistry. W. H Freeman and Co., Inc. New York pp. 554-560 and Sakakibara, J. et al. (1995) 270:17-20): acetate (from Acetyl-CoA)→3-hydoxy-3-methyl-glutaryl CoA→mevalonate→5-phosphomevalonate→5-pyrophosphomevalonate→isopentenyl pyrophosphate→dimethylallyl pyrophosphate→geranyl pyrophosphate→farnesyl pyrophosphate→squalene→squalene epoxide→lanosterol→cholesterol

While cholesterol is essential for the viability of eukaryotic cells, inordinately high serum cholesterol levels results in the formation of atherosclerotic plaques in the arteries of higher organisms. This deposition of highly insoluble lipid material onto the walls of essential blood vessels (e.g., coronary arteries) results in decreased blood flow and potential necrosis of the tissues deprived of adequate blood flow. HMG-CoA reductase is responsible for the conversion of 3-hydroxyl-3-methyl-glutaryl CoA (HMG-CoA) to mevalonate, which represents the first committed step in cholesterol biosynthesis. HMG-CoA is the target of a number of pharmaceutical compounds designed to lower plasma cholesterol levels. However, inhibition of MHG-CoA also results in the reduced synthesis of non-sterol intermediates (e.g., mevalonate) required for other biochemical pathways. SE catalyzes a rate-limiting reaction that occurs later in the sterol synthesis pathway and cholesterol in the only end product of the pathway following the step catalyzed by SE. As a result, SE is the ideal target for the design of anti-hyperlipidemic drugs that do not cause a reduction in other necessary intermediates (Nakamura, Y. et al. (1996) 271:8053-8056).

Epoxide Hydrolases

Epoxide hydrolases catalyze the addition of water to epoxide-containing compounds, thereby hydrolyting epoxides to their corresponding 1,2-diols. They are related to bacterial haloalkane dehalogenases and show sequence similarity to other members of the α/β hydrolase fold family of ernzes (e.g., bromoperoxidase A2 from Streptomyces aureofaciens, hydroxymuconic semialdehyde hydrolases from Pseudomonas putida, and haloalkane dehalogenase from Xanthobacter autotrophicus). Epoxide hydrolases are ubiquitous in nature and have been found in mammals, invertebrates, plants, fungi, and bacteria. This family of enzymes is important for the detoxification of xenobiotic epoxide compounds which are often highly electrophilic and destructive when introduced into an organism. Examples of epoxide hydrolase reactions include the hydrolysis of cis-9,10-epoxyoctadec-9(Z)-enoic acid (leukotoxin) to form its corresponding diol, threo-9,10-dihydroxyoctadec-12(Z)-enoic acid (leukotoxin diol), and the hydrolysis of cis-12,13-epoxyoctadec-9(Z)-enoic acid (isoleukotoxin) to form its corresponding diol threo-12,13-dihydroxyoctadec-9(Z)-enoic acid (isoleukotoxin diol). Leukotoxins alter membrane permeability and ion transport and cause inflammatory responses. In addition, epoxide carcinogens are known to be produced by cytochrome P450 as intermediates in the detoxification of drugs and environmental toxins.

The enzymes possess a catalytic triad composed of Asp (the nucleophile), Asp (the histidine-supporting acid), and His (the water-activating histidine). The reaction mechanism of epoxide hydrolase proceeds via a covalently bound ester intermediate initiated by the nucleophilic attack of one of the Asp residues on the primary carbon atom of the epoxide ring of the target molecule, leading to a covalently bound ester intermediate (Michael Arand, M. et al. (1996) J. Biol. Chem. 271:4223-4229; Rink, R et al. (1997) J. Biol chem. 272:14650-14657; Argiriadi, M. A. et al. (2000) J. Biol Chem. 275:15265-15270).

Catechol-O-Methyltransferase:

Catechol-O-methyltransferase (COMT) catalyzes the transfer of the methyl group of S-adenosyl-L-methionine (AdoMet; SAM) donor to one of the hydroxyl groups of the catechol substrate (e.g., L-dopa, dopamine, or DBA). Methylation of the 3′-hydroxyl group is favored over methylation of the 4′-hydroxyl group and the membrane bound isoform of COMT is more regiospecific than the soluble form. Translation of the soluble form of the enzyme results from utilization of an internal start codon in a full-length mRNA (1.5 kb) or from the translation of a shorter mRNA (1.3 kb), transcribed from an internal promoter. The proposed S_{N}2-like methylation reaction requires Mg⁺⁺ and is inhibited by Ca⁺⁺. The binding of the donor and substrate to COMT occurs sequentially. AdoMet first binds COMT in a Mg⁺⁺-independent manner, followed by the binding of Mg⁺⁺ and the binding of the catechol substrate.

The amount of COMT in tissues is relatively high compared to the amount of activity normally required, thus iribition is problematic. Nonetheless, inhibitors have been developed for in vitro use (e.g., gallates, tropolone, U-0521, and 3′,4′-dihydroxy-2-methyl-propiophetropolone) and for clinical use (e.g., nitrocatechol-based compounds and tolcapone). Administration of these inhibitors results in the increased half-life of L-dopa and the consequent formation of dopamine. Inhibition of COMT is also likely to increase the half-life of various other catechol-structure compounds, including but not limited to epinephrine/norepinephrine, isoprenaline, rimiterol, dobutamine, fenoldopam, apomorphine, and α-methyldopa. A deficiency in norepinephrine has been linked to clinical depression, hence the use of COMT uinibitors could be usefull in the treatment of depression. COMT inhibitors are generally well tolerated with minimal side effects and are ultimately metabolized in the liver with only minor accumulation of metabolites in the body (Männistö, P. T. and Kaakkola, S. (1999) Pharmacological Reviews 51:593-628).

The discovery of new drug metabolizing enzymes, and the polynucleotides encoding them, satisfies a need in the art by providing new compositions which are useful in the diagnosis, prevention, and treatment of autoimmuncfmflammatory, cell proliferative, developmental, endocrine, eye, metabolic, and gastrointestinal disorders, including liver disorders, and in the assessment of the effects of exogenous compounds on the expression of nucleic acid and amino acid sequences of drug metabolizing enzymes.

### SUMMARY OF THE INVENTION

The invention features purified polypeptides, drug metabolizing enzymes, referred to collectively as “DME” and individually as “DME-1,” “DME-2,” “DME-3,” “DME-4,” “DME-5,” “DME-6,” “DME-7,” “DME-8,” “DME-9,” “DME-10,” “DME-11, ” “DME-12,” “DME-13,” “DME-14,” “DME-15,” “DME-16,” “DME-17,” “DME-18,” and “DME-19.” In one aspect, the invention provides an isolated polypeptide selected from the group consisting of a) a polypeptide comprising an amino acid sequence selected from the group consisting of SEQ ID NO:1-19, b) a polypeptide comprising a naturally occurring amino acid sequence at least 90% identical to an amino acid sequence selected from the group consisting of SEQ ID NO:1-19, c) a biologically active fragment of a polypeptide having an amino acid sequence selected from the group consisting of SEQ ID NO:1-19, and d) an immunogenic fragment of a polypeptide having an amino acid sequence selected from the group consisting of SEQ ID NO:1-19. In one alternative, the invention provides an isolated polypeptide comprising the amino acid sequence of SEQ ID NO:1-19.

The invention further provides an isolated polynucleotide encoding a polypeptide selected from the group consisting of a) a polypeptide comprising an amino acid sequence selected from the group consisting of SEQ ID NO:1-19, b) a polypeptide comprising a naturally occurring amino acid sequence at least 90% identical to an amino acid sequence selected from the group consisting of SEQ ID NO:1-19, c) a biologically active fragment of a polypeptide having an amino acid sequence selected from the group consisting of SEQ ID NO:1-19, and d) an immunogenic fragment of a polypeptide having an amino acid sequence selected from the group consisting of SEQ ID NO:1-19. In one alternative, the polynucleotide encodes a polypeptide selected from the group consisting of SEQ ID NO:1-19. In another alternative, the polynucleotide is selected from the group consisting of SEQ ID NO:20-38.

Additionally, the invention provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-19, b) a polypeptide comprising a naturally occurring amino acid sequence at least 90%o identical to an amino acid sequence selected from the group consisting of SEQ TD NO:1-19, c) a biologically active fragment of a polypeptide having an amino acid sequence selected from the group consisting of SEQ ID NO: 1-19, and d) an immunogenic fragment of a polypeptide having an amino acid sequence selected from the group consisting of SEQ ID NO:1-19. In one alternative, the invention provides a cell transformed with the recombinant polynucleotide. In another alternative, the invention provides a transgenic organism comprising the recombinant polynucleotide.

The invention also provides a method for producing a polypeptide selected from the group consisting of a) a polypeptide comprising an amino acid sequence selected from the group consisting of SEQ ID NO:1-19, b) a polypeptide comprising a naturally occurring amino acid sequence at least 90% identical to an amino acid sequence selected from the group consisting of SEQ ID NO:1-19, c) a biologically active fragment of a polypeptide having an amino acid sequence selected from the group consisting of SEQ ID NO:1-19, and d) an immunogenic fragment of a polypeptide having an amino acid sequence selected from the group consisting of SEQ ID NO:1-19. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide selected from the group consisting of a) a polypeptide comprising an amino acid sequence selected from the group consisting of SEQ ID NO:1-19, b) a polypeptide comprising a naturally occurring amino acid sequence at least 90% identical to an amino acid sequence selected from the group consisting of SEQ ID NO:1-19, c) a biologically active fragment of a polypeptide having an amino acid sequence selected from the group consisting of SEQ ID NO:1-19, and d) an immunogenic fragment of a polypeptide having an amino acid sequence selected from the group consisting of SEQ ID NO:1-19.

The invention further provides an isolated polynucleotide selected from the group consisting of a) a polynucleotide comprising a polynucleotide sequence selected from the group consisting of SEQ ID NO:20-38, b) a polynucleotide comprising a naturally occurring polynucleotide sequence at least 90% identical to a polynucleotide sequence selected from the group consisting of SEQ ID NO:20-38, c) a polynucleotide complementary to the polynucleotide of a), d) a polynucleotide complementary to the polynucleotide of b), and e) an RNA equivalent of a)-d). In one alternative, the polynucleotide comprises at least 60 contiguous nucleotides.

Additionally, the invention provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:20-38, b) a polynucleotide comprising a naturally occurring polynucleotide sequence at least 90% identical to a polynucleotide sequence selected from the group consisting of SEQ ID NO:20-38, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex, and optionally, if present, the amount thereof. In one alternative, the probe comprises at least 60 contiguous nucleotides.

The invention further provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:20-38, b) a polynucleotide comprising a naturally occurring polynucleotide sequence at least 90% identical to a polynucleotide sequence selected from the group consisting of SEQ ID NO:20-38, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof, and, optionally, if present, the amount thereof.

The invention further provides a composition comprising an effective amount of a polypeptide selected from the group consisting of a) a polypeptide comprising an amino acid sequence selected from the group consisting of SEQ ID NO:1-19, b) a polypeptide comprising a naturally occurring amino acid sequence at least 90% identical to an amino acid sequence selected from the group consisting of SEQ ID NO:1-19, c) a biologically active fragment of a polypeptide having an amino acid sequence selected from the group consisting of SEQ ID NO:1-19, and d) an immunogenic fragment of a polypeptide having an amino acid sequence selected from the group consisting of SEQ ID NO:1-19, and a pharmaceutically acceptable excipient. In one embodiment, the composition comprises an amino acid sequence selected from the group consisting of SEQ ID NO:1-19. The invention additionally provides a method of treating a disease or condition associated with decreased expression of functional DME, comprising administering to a patient in need of such treatment the composition.

The invention also provides a method for screening a compound for effectiveness as an agonist of a polypeptide selected from the group consisting of a) a polypeptide comprising an amino acid sequence selected from the group consisting of SEQ ID NO:1-19, b) a polypeptide comprising a naturally occurring amino acid sequence at least 90% identical to an amino acid sequence selected from the group consisting of SEQ ID NO:1-19, c) a biologically active fragment of a polypeptide having an amino acid sequence selected from the group consisting of SEQ ID NO:1-19, and d) an immunogenic fragment of a polypeptide having an amino acid sequence selected from the group consisting of SEQ ID NO:1-19. The method comprises a) exposing a sample comprising the polypeptide to a compound, and b) detecting agonist activity in the sample. In one alternative, the invention provides a composition comprising an agonist compound identified by the method and a pharmaceutically acceptable excipient. In another alternative, the invention provides a method of treating a disease or condition associated with decreased expression of functional DME, comprising administering to a patient in need of such treatment the composition.

Additionally, the invention provides a method for screening a compound for effectiveness as an antagonist of a polypeptide selected from the group consisting of a) a polypeptide comprising an amino acid sequence selected from the group consisting of SEQ ID NO:1-19, b) a polypeptide comprising a naturally occurring amino acid sequence at least 90% identical to an amino acid sequence selected from the group consisting of SEQ ID NO:1-19, c) a biologically active fragment of a polypeptide having an amino acid sequence selected from the group consisting of SEQ ID NO:1-19, and d) an immunogenic fragment of a polypeptide having an amino acid sequence selected from the group consisting of SEQ ID NO:1-19. The method comprises a) exposing a sample comprising the polypeptide to a compound, and b) detecting antagonist activity in the sample. In one alternative, the invention provides a composition comprising an antagonist compound identified by the method and a pharmaceutically acceptable excipient. In another alternative, the invention provides a method of treating a disease or condition associated with overexpression of functional DME, comprising administering to a patient in need of such treatment the composition.

The invention further provides a method of screening for a compound that specifically binds to a polypeptide selected from the group consisting of a).a polypeptide comprising an amino acid sequence selected from the group consisting of SEQ ID NO:1-19, b) a polypeptide comprising a naturally occurring amino acid sequence at least 90% identical to an amino acid sequence selected from the group consisting of SEQ ID NO:1-19, c) a biologically active fragment of a polypeptide having an amino acid sequence selected from the group consisting of SEQ ID NO:1-19, and d) an immunogenic fragment of a polypeptide having an amino acid sequence selected from the group consisting of SEQ ID NO:1-19. The method comprises a) combining the polypeptide with at least one test compound under suitable conditions, and b) detecting binding of the polypeptide to the test compound, thereby identifying a compound that specifically binds to the polypeptide.

The invention further provides a method of screening for a compound that modulates the activity of a polypeptide selected from the group consisting of a) a polypeptide comprising an amino acid sequence selected from the group consisting of SEQ ID NO:1-19, b) a polypeptide comprising a naturally occurring amino acid sequence at least 90% identical to an amino acid sequence selected from the group consisting of SEQ ID NO:1-19, c) a biologically active fragment of a polypeptide having an amino acid sequence selected from the group consisting of SEQ ID NO:1-19, and d) an imniunogenic fragment of a polypeptide having an amino acid sequence selected from the group consisting of SEQ ID NO:1-19. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

The invention further provides a method for screening a compound for effectiveness in altering expression of a target polynucleotide, wherein said target polynucleotide comprises a polynucleotide sequence selected from the group consisting of SEQ ID NO:20-38, the method comprising a) exposing a sample comprising the target polynucleotide to a compound, and b) detecting altered expression of the target polynucleotide.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:20-38, ii) a polynucleotide comprising a naturally occurring polynucleotide sequence at least 90% identical to a polynucleotide sequence selected from the group consisting of SEQ ID NO:20-38, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:20-38, ii) a polynucleotide comprising a naturally occurring polynucleotide sequence at least 90% identical to a polynucleotide sequence selected from the group consisting of SEQ ID NO:20-38, iii) a polynucleotide complementary to the polynucleotide of i), iv) a polynucleotide complementary to the polynucleotide of ii), and v) an RNA equivalent of i)-iv). Alternatively, the target polynucleotide comprises a fragment of a polynucleotide sequence selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for the full length polynucleotide and polypeptide sequences of the present invention.

Table 2 shows the GenBank identification number and annotation of the nearest GenBank homolog for polypeptides of the invention. The probability score for the match between each polypeptide and its GenBank homolog is also shown.

Table 3 shows structural features of polypeptide sequences of the invention, including predicted motifs and domains, along with the methods, algorithms, and searchable databases used for analysis of the polypeptides.

Table 4 lists the cDNA and/or genomic DNA fragments which were used to assemble polynucleotide sequences of the invention, along with selected fragments of the polynucleotide sequences.

Table 5 shows the representative cDNA library for polynucleotides of the invention.

Table 6 provides an appendix which describes the tissues and vectors used for construction of the cDNA libraries shown in Table 5.

Table 7 shows the tools, programs, and algorithms used to analyze the polynucleotides and polypeptides of the invention, along with applicable descriptions, references, and threshold parameters.

### DESCRIPTION OF THE INVENTION

Before the present proteins, nucleotide sequences, and methods are described, it is understood that this invention is not limited to the particular machines, materials and methods descnbed, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a phlrality of such host cells, and a reference to “an antibody” is a reference to one or more antibodies and equivalents thereof known to those skilled in the art, andso forth.

Unless defined otherwise, all tecnical and scientific terms used herein have the sarme meanings as commonly understood by one of ordinary skill in the art to which this invention belongs. Although any machines, materials, and methods similar or equivalent to those described herein can be used to practice or test the present invention, the preferred machines, materials and methods are n w described. All publications mentioned herein are cited for the purpose of descnbing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“DME” refers to the amino acid sequences of substantially purified DME obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of DME. Agonists may include proteins, nucleic acids, carbohydrates, small molecules, or any other compound or composition which modulates the activity of DME either by directly interacting with DME or by acting on components of the biological pathway in which DME participates.

An “allelic variant” is an alternative form of the gene encoding DME. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding DME include those sequences with deletions, insertions, or substitutions of different nucleotides, resulting in a polypeptide the same as DME or a polypeptide with at least one functional characteristic of DME. Included within this definition are polymorphisms which may or may not be readily detectable using a particular oligonucleotide probe of the polynucleotide encoding DME, and improper or unexpected hybridization to allelic variants, with a locus other than the normal chromosomal locus for the polynucleotide sequence encoding DME. The encoded protein may also be “altered,” and may contain deletions, insertions, or substitutions of anuno acid residues which produce a silent change and result in a functionally equivalent DME. Deliberate amino acid substitutions may be made on the basis of similarity in polarity, charge, solubility, hydrophobicity, hydrophilicity, and/or the amphipathic nature of the residues, as long as the biological or immunological activity of DME is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of DME. Antagonists may include proteins such as antibodies, nucleic acids, carbohydrates, small molecules, or any other compound or composition which modulates the activity of DME either by directly interacting with DME or by acting on components of the biological pathway in which DME participates.

The term “antibody” refers to intact immunoglobulin molecules as well as to fragments thereof, such as Fab, F(ab′)₂, and Fv fragments, which are capable of binding an epitopic determinant Antibodies that bind DME polypeptides can be prepared using intact polypeptides or using fragments containing srnall peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KIH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenicdeterminant may compete with the intact antigen (i.e., the immiunogen used to elicit the immune response) for binding to an antibody.

The term “antisense” refers to any composition capable of base-pairing with the “sense” (coding) strand of a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic DME, or of any oligopeptide thereof, to induce a specific immune response in appropriate animals or cells and to bind with specific antibodies.

“Complementary” describes the relationship between two single-stranded nucleic acid sequences that. anneal by base-pairing. For example, 5′-AGT-3′ pairs with its complement, 3′-TCA-5′.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding DME or fragments of DME may be employed as hybridization probes. The probes may be stored in freezeried form and may be associated with a stabilizing agent such as a carbohydrate. In hybridizations, the probe may be deployed in an aqueous solution containing salts (e.g., NaCI), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genornic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Mg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polnucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable laber” refers to a reporter molecule or enzyrne that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortinent of stable substructures, thus allowing acceleration of the evolution of new protein functions.

A “fragment” is a unique portion of DME or the polynucleotide encoding DME which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, maybe at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:20-38 comprises a region of unique polynucleotide sequence that specifically identifies SEQ ID NO:20-38, for example, as distinct from any other sequence in the genome from which the fragment was obtained. A fragment of SEQ ID NO:20-38 is useful, for example, in hybridization and amplification technologies and in analogous methods that distinguish SEQ ID NO:20-38 from related polynucleotide sequences. The precise length of a fragment of SEQ ID NO:20-38 and the region of SEQ ID NO:20-38 to which the fragment corresponds are routinely determinable by one of ordinary sklll in the art based on the intended purpose for the fragment.

A fragment of SEQ ID NO:1-19 is encoded by a fragment of SEQ ID NO:20-38. A fragment of SEQ ID NO:1-19 comprises a region of unique amino acid sequence that specifically identifies SEQ ID NO:1-19. For example, a fragment of SEQ ID NO:1-19 is useful as an immunogenic peptide for the development of antibodies that specifically recognize SEQ ID NO:1-19. The precise length of a fragment of SEQ ID NO:1-19 and the region of SEQ ID NO:1-19 to which the fragment corresponds are routinely determinable by one of ordinary skill in the artbased on the intended purpose for the fragment

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison WI). CLUSTAL V is described in Higgins, D.G. and-P.M. Sharp (1989) CABIOS 5:151-153 and in Higgins, D.G. et al. (1992) CABIOS 8:189-191. For pairwise alignments of polynucleotide sequences, the default parameters are set as follows: Ktuple=2, gap penalty=S, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403410), which is available from several sources, including the NCBI, Bethesda, Md., and on the Internet at http:/Iwww.ncbi.nnih gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlnm igov/gor/bl2.htmrl. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:

Matrix: BLOSUM62

Rewardfor match: 1

Peiaity for nismnatch:—2

Open Gap:5 and Extension Gap:2 penalties

Gap x diop-off:50

Expect:10

Word Size:11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 10Q, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (descmbed and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

Matrix: BLOSUM62

Open. Gap:11 and Exteitsioit Gap: 1 penalties

Gap x drop-off 50

Expect:10

Word Size:3

Filter: on

Percent identity may be measured over the length of an entire defined polypeptide sequence, for example, as defined by a particular SEQ ID NO, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, maybe used to describe a length over which percentage identity may be measured.

“Human artificial chromosomes” (CACs) are linear ricrochromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in detetmining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and maybe consistent among hybridization experiments, whereas wash conditions maybe varied among experiments to achieve the desired stringency, and therefore hybridization specificity. Permissive annealing conditions occur, for example, at 68° C. in the presence of about 6×SSC, about 1% (wtv) SDS, and about 100 μg/m sheared, denatured salmon sperm DNA

Generally, stringency of hybridization is expressed, in part, with reference to the temperature under which the wash step is carried out Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambroolk, J. et al. (1989) Molecular Cloning: A Laboratory.Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hyoridization complex” refers to a complex formed between two nucleic acid sequences by virtue of the formation of hydrogen bonds between complementary bases. A hybridization complex maybe formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of DME which is capable of eliciting an immune response when introduced into a living organism, for example, a mammal. The term “immunogenic fragment” also includes any polypeptide or oligopeptide fragment of DME which is useful in any of the antibody production methods disclosed herein or known in the art.

The term “microarray” refers to an arrangement of a plurality of polynucleotides, polypeptides, or other chemical compounds on a substrate.

The terms “element” and “array element” refer to a polynucleotide, polypeptide, or other chemical compound having a unique and defined position on a microarray.

The term “modulate” refers to a change in the activity of DME. For example, modulation may cause an increase or a decrease in protein activity, binding characteristics, or any other biological, functional, or immunological properties of DME.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an DME may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of DME.

“Probe” refers to nucleic acid sequences encoding DME, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, cheni lninescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, maybe used.

Methods for preparing and using probes and primers are descnbed in the references, for example Sambrook, J. et al. (1989) Molecular Cloning: A Laboratory Manual, 2^{nd} ed., vol. 1-3, Cold Spring Harbor Press, Plainview N.Y.; Ausubel, F. M. et al. (1987) Current Protocols in Molecular Biology, Greene Publ. Assoc. & Wldey-Intersciences, New York N.Y.; Inis, M. et al. (1990) PCR Protocols. A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tes.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regi ns of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that. could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, cheniluminescent, or chromogenic agents; substrates; cofactors; inhibitors; magnetic particles; and other moieties known in the art.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of nbose instead of deoxyribose.

The term “sample” is used in its broadest sense. A sample suspected of containing DME, nucleic acids encoding DME, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” refers to the collective pattern of gene expression by a particular cell type or tissue under given conditions at a given time.

“Transformatior” describes a process by which exogenous DNA is introduced into a recipient cell. Transformation may occur under natural or artificial conditions according to various methods well known in the art, and may rely.on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and anrimals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be descnbed as, for example, an “allelic” (as defined above), “splice,” “species,” or “polymorphic” variant. A splice variant may have significant identity to a reference molecule, but will generally have a greater or lesser number of polynucleotides due to alternate splicing of exons during MRNA processing. The corresponding polypeptide may possess additional functional domains or lack domains that are present in the reference molecule. Species variants are polynucleotide sequences that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

### THE INVENTION

The invention is based on the discovery of new human drug metabolizing enzymes (DME), the polynucleotides encoding DME, and the use of these compositions for the diagnosis, treatment, or prevention of autoimmunefmflammatory, cell proliferative, developmental, endocrine, eye, metabolic, and gastrointestinal disorders, including liver disorders.

Table 1 summarizes the nomenclature for the full length polynucleotide and polypeptide sequences of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incytz Polynucleotide ID) as shown.

Table 2 shows sequences with homology to the polypeptides of the invention as identified by BLAST analysis against the GenBank protein (genpept) database. Columns 1 and 2 show the polypeptide sequence identification number (Polypeptide SEQ ED NO:) and the corresponding Incyte polypeptide sequence number (Incyte Polypeptide ID) for polypeptides of the invention. Colbn=3 shows the GenBank identification number (Genbank ID NO:) of the nearest GenBank homolog. Column 4 shows the probability score for the match between each polypeptide and its GenBank homolog. Column S shows the annotation of the GenBank homolog along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Cohlmn 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are drug metabolizing enzymes. For example, SEQ ID NO:3 is 40% identical to a mouse cytochrome P450 monooxygenase (GenBank ID g2653663) as determined by the Basic Local Alignment Search Tool (BLAST, see Table 2). The BLAST probability score is 5.3e-91, which indicates the probability of obtaining the observed polypeptide sequence alignment by chance. SEQ ID NO:3 also contains cytochrome P450 signature sequences as determined by searching for statistically significant matches in the hidden Markov model (HMM)-based PFAM database of conserved protein family domains (see Table 3). Data from BLIMPS and PROFILESCAN analyses provide further corroborative evidence that SEQ ID NO:3 is a member of the cytochrome P450 family.

In an alternative example, SEQ ID NO:1 is 58% identical to a lysyl oxidase from the yellow perch (erca flavescens; GenBank ID g4929199) as determined by BLAST analysis The BLAST probability score is 1.9e-248. SEQ ID NO:1 also contains cytochrome P450 signature sequences as determined by searching for statistically significant matches in the HMM-based PFAM database of conserved protein family domains and by BLIMPS analyses.

In an alternative example, SEQ ID NO:2 is 61% identical to human flavin-containing monooxygenase 5 (GnBank ID g559046) as determined by BLAST analysis, with a probability score of 4.5e-181. SEQ ID NO:2 also contains flavin-containing monooxygenase signature sequences as determined by searching for statistically significant matches in the BHM-based PFAM database of conserved protein family domains and by BLIMPS and PROFILESCAN analyses.

In an alternative example, SEQ ID NO:4 is 39% identical to a Pseudomonas 2,3-butanediol dehydrogenase (GenBank ID g529564) as determined by BLAST analysis, with a probability score of 2.0e-61. SEQ ID NO:4 also contains dehydrogenase signature sequences as determined by searching. for statistically significant matches in the HMM-based PFAM database of conserved protein family domains. Data from BUWS, MOTIFS, and PROFHESCAN analyses provide further corroborative evidence that SEQ ID NO:4 is a dehydrogenase.

SEQ ID NO:5 is 54% identical to a Bacillus quinone oxidase (GenBank ID g2633069) as determined by BLAST analysis, with a probability score of 7.1e-96. Data obtained by searching the HMM-based PFAM database of conserved protein family domains and by BLIMPS analyses provide further corroborative evidence that SEQ ID NO:5 is a quinone oxidase.

In an alternative example, SEQ ID NO:6 is 92% identical to mouse heparan sulfate 6-sulfotransferase 2 (GenBank ID g6683558) as determined by BLAST analysis, with a probability score of 2.3e-255.

In an alternative example, SEQ ID NO:7 is 90% identical to a human glutathione S-transferase subunit (GenBank ID g242749) as determined by BLAST analysis, with a probability score of 1.3e-101. SEQ ID NO:7 also contains glutathione S-transferase signature sequences as determined by searching for statistically significant matches in the M-based PFAM database of conserved protein family domains and by BIMPS analyses.

In an alternative example, SEQ ID NO:8 is 40% identical to a human steriod dehydrogenase (GenBank ID g5531815) as determined by BLAST analysis, with a probability score of l.9e-56. SEQ ID NO:8 also contains dehydrogenase signature sequences as determined by searching for statistically significant matches in the HMM-based PFAM database of conserved protein family domains.

SEQ ID NO:9 is 47% identical to a rabbit liver carboxylesterase (GenBank ID g3219695) as determined by the Basic Local Alignment Search Tool (BLAST). (See Table 2.) The BLAST probability score is 6.3e-72, which indicates the probability of obtaining the observed polypeptide sequence alignment by chance. SEQ ID NO:9 also contains carboxylesterase domains as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO:9 is a carboxylesterase.

SEQ ID NO:10 is 45% identical to human carboxylesterase (GenBank ID g180950) as determined by the Basic Local Alignment Search Tool (BLAST, see Table 2). The BLAST probability score is 8.7e-130, which indicates the probability of obtaining the observed polypeptide sequence alignment by chance. SEQIID NO:10 also contains carboxylesterase domains as determined by searching for statistically significant matches in the hidden Markov model (BMM based PFAM database of conserved protein family domains (see Table 3). Data from BUWNS, MOTIFS, and PROFILESCAN analyses provide further corroborative evidence that SEQ ID NO:10 is a carboxylesterase.

In an alternative example, SEQ ID NO:11 is 89% identical to murine heparan sulfate 6-sulfotransferase 2 (GenBank ID g6683558) as determined by the Basic Local Alignment Search Tool (BLAST, see Table 2). The BLAST probability score is 1.8e-236, which indicates the probability of obtaining the observed polypeptide sequence alignment by chance, and provides evidence that SEQ ID NO:11 is a DME, and specifically that SEQ ID NO:11 is a sulfotransferase.

In an alternative example, SEQ ID NO:12 is 25% identical to a Bacillus subtilis epoxide hydrolase (GenBank ID g2633182) as determined by the Basic Local Alignment Search Tool (BLAST, see Table 2). The BLAST probability score is 1.3e-11, which indicates the probability of obtaining the observed polypeptide sequence alignment by chance. SEQ ID NO:12 also contains hydrolase domains as determined by searching for statistically significant matches in the hidden Markov model (1M)-based PFAM database of conserved protein family domains (see Table 3). Data from BLIMPS analyses provide further corroborative evidence that SEQ ID NO:12 is a hydrolase.

In an alternative example, SEQ ID NO:13 is 83 % identical to a rat beta-alanine-pyruvate aminotransferase (GenBank ID g1944136) as determined by the BLAST analysis (see Table 2). The BLAST probability score is 1.1e-234. SEQ ID NO:13 also contains aminotransferase domains as determined by searching for statistically significant matches in the hidden Markov model (HMM)-based PFAM database of conserved protein family domains (see Table 3). Data from BLMPS and PROFILESCAN analyses provide further corroborative evidence that SEQ ID NO:13 is an arninotransferase.

In an alternative example, SEQ ID NO:14 is 50% identical to a guinea pig hyroxysteroid sulfotransferase (GnBank ID g1151081) as determined by the BLAST analysis (see Table 2). The BLAST probability score is 5.4e-34, and provides evidence that SEQ ID NO:14 is a sulfotransferase.

In an alternative example, SEQ ID NO:15 is 52% identical to a guinea pig copper/zinc superoxide dismutase (GenBank ID g1066120) as determined by the BLAST analysis (see Table 2). The BLAST probability score is 2.1e-25. SEQ ID NO:15 also contains copper/zinc superoxide dismutase domains as determined by searching for statistically significant matches in the hidden Markov model (HMM)based PFAM database of conserved protein family domains (see Table 3>. Data from BLMPS analyses provide further corroborative evidence that SEQ ID NO:15 is a copper/zinc superoxide dismutase.

SEQ ID NO:16 is 37%.identical to human 3′-phosphoadenylylsulfate-galactosylceramide 3′-sulfotransferase (cerebroside sulfotransferase, GenBank ID g1871141) as determined by the Basic Local Alignment Search Tool (BLAST, see Table 2). The BLAST probability score is 2.8e-60, which indicates the probability of obtaining the observed polypeptide sequence alignment by chance.

In an alternative example, SEQ ID NO:17 is 38% identical to a putative C. elegans monoamine oxidase (GenBank ID g6782275) as determined by BLAST analysis with a probability score of 3.0e-99. SEQ ID NO:17 also contains a monoamine oxidase domain as determined by searching for statistically significant matches in the hidden Markov model (HMM)-based PFAM database of conserved protein family domains (see Table 3). Data from BLIMPS analysis provide further corroborative evidence that SEQ ID NO:17 is a monoamine oxidase.

In an alternative example, SEQ ID NO:18 is 36% identical to human catechol-O-methyltransferase (GenBank ID g179955) as determined by BLAST analysis with a probability score of 9.5e41. SEQ ID NO:18 is also 36% identical to murine catechol-O-methyltransferase (GenBank ID g3493253) as determined by BLAST analysis with a probability score of 1.3e41.

In an alternative example, SEQ ID NO:19 is 44% identical to Fundulus heteroclitus cytochrome P450 2N1 (GenBank ID g5852342) as determined by the Basic Local Alignment Search Tool (BLAST, see Table 2). The BLAST probability score is 4.6e-99, which indicates the probability of obtaining the observed polypeptide sequence alignment by chance. SEQ ID NO:19 also contains cytochrome P450 domains as determined by searching for statistically significant matches in the hidden Markov model (HMM)-based PFAM database of conserved protein family domains (see Table 3). Data from BLIMPS, MOTIFS, and PROFELESCAN analyses provide further corroborative evidence that SEQ ID NO:19 is a cytochrome P450.

The algorithms and parameters for the analysis of SEQ ID NO:1-19 are described in Table 7.

As shown in Table 4, the full length polynucleotide sequences of the present invention were assembled using cDNA sequences or coding (exon) sequences derived from genomic DNA, or any combination of these two types of sequences. Columns 1 and 2 list the polynucleotide sequence identification number (Polynucleotide SEQ ID NO:) and the corresponding Incyte polynucleotide consensus sequence number (Incyte Polynucleotide ID) for each polynucleotide of the invention. Column 3 shows the length of each polynucleotide sequence in basepairs. Column 4 lists fragments of the polynucleotide sequences which are useful, for example, in hybridization or amplification technologies that identify SEQ ID NO:20-38 or that distinguish between SEQ ID NO:20-3 8 and related polynucleotide sequences. Column 5 shows identification numbers corresponding to cDNA sequences, coding sequences (exons) predicted from genomic DNA, and/or sequence assemblages comprised of both cDNA and genomic DNA. These sequences were used to assemble the full length polynucleotide sequences of the invention. Columns 6 and 7 of Table 4 show the nucleotide start (5′) and.stop (3′) positions of the cDNA and/or genomic sequences in column 5 relative to their respective full length sequences.

The identification numbers in Column 5 of Table 4 may refer specifically, for example, to Incyte cDNAs along with their corresponding cDNA libraries. For example, 7690384J1 is the identification number of an Incyte cDNA sequence, and PROSTMEO6 is the cDNA library from which it is derived. Incyte cDNAs for which cDNA libraries are not indicated were derived from pooled cDNA libraries (e.g., 55017748J1). Alternatively, the identification numbers in column 5 may refer to GenBank cDNAs or ESTs (e.g., g1203094) which contn1buted to the assembly of the full length polynucleotide sequences. In addition, the identification numbers in colmn 5 may identify sequences derived from the ENSEMBl. (The Sanger Centre, Cambridge, UK) database (Le., those sequences including the designation “ENST”). Alternatively, the identification numbers in cohlmn 5 maybe derived from the NCBI RefSeq Nucleotide Sequence Records Database (Le., those sequences including the designation “NM” or “NT”) or the NCBI RefSeq Protein Sequence Records (i.e., those sequences including the designation “NP”). Alternatively, the identification numbers in column 5 may refer to assemblages of both cDNA and Genscan-predicted exons brought together by an “exon stitching” algorithm. For example, FL_XXXXXX_N_{1—}N_{2—}YYYYY_N_{3—}N₄ represents a “stitched” sequence in which XXXXXX is the identification number of the cluster of sequences to which the algorithm was applied, and YYYYYis the number of the prediction generated by the algorithm, and N_{1,2,3, . . .} , if present, represent specific exons that may have been manually edited during analysis (See Example V). Alternatively, the identification numbers in column 5 may refer to assemblages of exons brought together by an “exon-stretching” algorithm. For example, FLXXXXXX_gAAAAA_gBBBBB_{—}1_N is the identification number of a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenPank identifier (i.e., gBBBBB).

Alternatively, a prefix identifies component sequences that were hand-edited, predicted from genoric DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, | Exon prediction from genomic sequences using, for |
| ENST | example, |
| | GENSCAN (Stanford University, CA, USA) or FGENES |
| | (Computer Genomics Group, The Sanger Centre, |
| | Cambridge, UK). |
| GBI | Hand-edited analysis of genomic sequences. |
| FL | Stitched or stretched genomic sequences (see Example V). |
| INCY | Full length transcript and exon prediction from mapping |
| | of EST sequences to the genome. Genomic location and |
| | EST composition data are combined to predict the exons |
| | and resulting transcript. |

In some cases, Incyte cDNA coverage redundant with the sequence coverage shown in column 5 was obtained to confirm the final consensus polynucleotide sequence, but the relevant Incyte cDNA identification numbers are not shown

Table 5 shows the representative cDNA libraries for those full length polynucleotide sequences which were assembled using Incyte cDNA sequences. The representative cDNA library is the Incyte cDNA library which is most frequently represented by the Incyte cDNA sequences which were used to assemble and confirm the above polynucleotide sequences. The tissues and vectors which were used to construct the cDNA libraries shown in Table 5 are described in Table 6.

The invention also encompasses DME variants. A preferred DME variant is one which has at least about 80%, or alternatively at least about 90%, or even at least about 95% amino acid sequence identity to the DME amino acid sequence, and which contains at least one functional or structural characteristic of DME.

The invention also encompasses polynucleotides which encode DME. In a particular embodiment, the invention encompasses a polynucleotide sequence comprising a sequence selected from the group consisting of SEQ ID NO:20-38, which encodes DME. The polynucleotide sequences of SEQ ID NO:20-3 8, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses a variant of a polynucleotide sequence encoding DME. In particular, such a variant polynucleotide sequence will have at least about 70%, or alternatively at least about 85%, or even at least about 95% polynucleotide sequence identity to the polynucleotide sequence encoding DME. A particular aspect of the invention encompasses a variant of a polynucleotide sequence comprising a sequence selected from the group consisting of SEQ ID NO:20-38 which has at least about 70%, or alternatively at least about 85%, or even at least about 95% polynucleotide sequence identity to a nucleic acid sequence selected from the group consisting of SEQ ID NO:20-38. Any one of the polynucleotide variants descnbed above can encode an amino acid sequence which contains at least one functional or structural characteristic of DME.

It will be appreciated by those skilled in the art that as a result of the degeneracy of the genetic code, a multitude of polynucleotide sequences encoding DME, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring DME, and all such variations are to be considered as being specifically disclosed.

Although nucleotide sequences which encode DME and its variants are generally capable of hybridizing to the nucleotide sequence of the naturally occurring DME under appropriately selected conditions of stringency, it may be advantageous to produce nucleotide sequences encoding DME or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding DME and its derivatives without altering the encoded amino acid sequences include the production of RNA transcripts having more desirable properties, such as a greater half-life, than transcripts produced from the naturally occurring sequence.

The invention also encompasses production of DNA sequences which encode DME and DME derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding DME or any fragment thereof.

Also encompassed by the invention are polynucleotide sequences that are capable of hybridizing to the claimed polynucleotide sequences, and, in particular, to those shown in SEQ ID NO:20-38 and fragments thereof under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosysterns). Sequencing is then carried out using either the ABI 373 or 377 DNA sequencing system (Applied Biosystems), the MEGABACE 1000 DNA sequencing system (Molecular Dynamics, Sunnyvale Calif.), or other systems known in the art. The resulting sequences are analyzed using a variety of algorithms which are well known in the art (See, e.g., Ausubel, F. M. (1997) Short Protocols in Molecular Biology, John Wiley & Sons, New York N.Y., unit 7.7; Meyers, PA. (1995) Molecular Biology and Biotechnoloay, Wiley VCH, New York N.Y., pp. 856-853.)

The nucleic acid sequences encoding DME may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, Tₘ et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcnbed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity maybe converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode DME may be cloned in recombinant DNA molecules that direct expression of DNE, or fragments or functional equivalents thereof, in appropriate host cells. Due to the inherent degeneracy of the genetic code, other DNA sequences which encode substantially the same or a functionally equivalent amino acid sequence may be produced and used to express DME.

The nucleotide sequences of the present invention can be engineered using methods generally known in the art in order to alter DME-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULAPBREEDING (Maxygen Inc., Santa Clara Calif.; descnbed in U.S. Pat. No. 5,837,458; Chang, C. -C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of DME, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and furter subjected to recursive rounds of DNA shuffling and selection/screening. Thus, genetic diversity is created through “artificiar” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maxinling the genetic diversity of multiple naturally occurring genes in a directed and controllable manner.

In another embodiment, sequences encoding DME may be synthesized, in whole or in part, using chemical methods well known in the arl. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, DME itself or a fragment thereof may be synthesized using chemical methods. For example, peptide synthesis can be performed using various solution-phase or solid-phase techniques. (See, e.g., Creighton, T. (1984) Proteins, Structures and Molecular Properties, W H Freeman, New York N.Y., pp. 55-60; and Roberge, J. Y. et al. (1995) Science 269:202-204.) Automated synthesis maybe achieved using the ABI 431A peptide synthesizer (Applied Biosystems). Additionally, the amino acid sequence of DME, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active DME, the nucleotide sequences encoding DME or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding DME. Such elements may vary in their strength and specificity. Specific initiation signals may also be used to achieve more efficient translation of sequences encoding DME. Such signals include the ATG initiation codon and adjacent sequences, e.g. the Kozak sequence. In cases where sequences encoding DME and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results ProbL Cell Differ. 20:125-162.)

Methods which are well known to those skilled in the art may be used to construct expression vectors containing sequences encoding DME and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel. F. M. et al. (1995) Current Protocols in Molecular Biology, John Vrley & Sons, New York N.Y., ch 9, 13, and 16.)

A variety of expression vector/host systems may be utilized to contain and express sequences encoding DMB. These include, but are not limited to, mnicroorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hil Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Imnunol. 31(3):219-226; and Verma, I. M. and N. Somia (1997) Nature 389:239-242.) The inventionis not limited by the host cell employed.

In bacterial systems, a number of cloning and expression vectors may be selected depending upon the use intended for polynucleotide sequences encoding DME. For example, routine cloning, subcloning, and propagation of polynucleotide sequences encoding DME can be achieved using a multifunctional E. coli vector such as PBLUESCRIPT (Stratagene, La Jolla Calif.) or PSPORT1 plasmid (Life Technologies). Ligation of sequences encoding DME into the vector's multiple cloning site disrupts the. lacZ gene, allowing a colorimetric screening procedure for identification of transformed bacteria containing recombinant molecules. In addition, these vectors maybe useful for in vitro transcription, dideoxy sequencing, single strand rescue with helper phage, and creation of nested deletions in the cloned sequence. (See, e.g., Van Heeke, G. and S. M. Schuster (1989) J. BioL Chem. 264:5503-5509.) When large quantities of DME are needed, e.g. for the production of antibodies, vectors which direct high level expression of DME may be used. For example, vectors containing the strong, inducible SP6 or T7 bacteriophage promoter may be used.

Yeast expression systems may be used for production of DME. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Biofrechnology 12:181-184.)

Plant systems may also be used for expression of DME. Transcription of sequences encoding DME maybe drivenby viral promoters, e.g., the 35S and 19S promoters of CaMVused alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, sequences encoding DME may be ligated into an adenovirus transcription/translation complex consisting of the late promoter and tripartite leader sequence. Insertion in a non-essential El or E3 region of the viral genome may be used to obtain infective virus which expresses DME in host cells. (See, e.g., Logan, J. and Tₘ Shenk (1984) Proc. And. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Hariington, J. J. et al. (1997) Nat. Genet. 15:345-355.)

For long term production of recombinant proteins in mammalian systems, stable expression of DME in cell lines is preferred. For example, sequences encoding DME can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G418; and als and pat confer resistance to chlorsulfuron and phosphinotricin acetyltransferase, respectively. (See, e.g., Wigler, M. et al. (1980) Proc. Natl. Acad. Sci. USA 77:3567-3570; ColberGarapin, F. et al. (1981) J. Mol Biol. 150:1-14.) Additional selectable genes have been described, e.g., trpB and hisD, which alter cellular requirements for metabolites. (See, e.g., Hartman, S. C. and R. C. Mulligan (1988) Proc. Natl Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attnbutable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may -need to be confirmed. For example, if the sequence encoding DME is inserted within a marker gene sequence, transformed cells containing sequences encoding DME can be identified by the absence of marker gene finction. Alternatively, a marker gene can be placed in tandem with a sequence encoding DME under the control of a single promoter. Expression of the marker gene in response to induction or selection usually indicates expression of the tandem gene as well

In general, host cells that contain the nucleic acid sequence encoding DME and that express DMB maybe identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of DMB using either specific polyclonal or monoclonal antabodies are known in the art Examples of such techniques include enzyme-linked immunosorbent assays (ELISAs), radioinmmunoassays (RIAs), and fluorescence activated cell sorting (FACS). A two-site, monoclonal-based immunoassay utilizing monoclonal antibodies reactive to two non-interfering epitopes on DME is preferred, but a competitive binding assay may be employed. These and other assays are well known in the art. (See, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and maybe used in various nucleic acid and amino acid assays. Means for producing labeled hybridization or PCR probes for detecting sequences related to polynucleotides encoding DME include oligolabeling, nick traslation, end-labeling, or PCR amplification using a labeled nucleotide. Alternatively, the sequences encoding DME, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison WI), and US Biochemical. Suitable reporter molecules or labels which may be used for ease of detection include radionuclides, enzymes, fluorescent, chemiluminescent, or chromogenic agents, as well as substrates, cofactors, inlnbitors, magnetic particles, and the like.

Host cells transformed with nucleotide sequences encoding DME may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode DME maybe designed to contain signal sequences which direct secretion of DME through a prokaryotic or eukaryotic cell membrane.

In addition, a host cell strain maybe chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and W138) are available from the American Type Culture Collection (ATCC, Manassas Va.) and may be chosen to ensure the correct modification and processing of the foreign protein.

In another embodiment of the invention, nataral modified, or recombinant nucleic acid sequences encoding DME may be ligated to a heterologous sequence resulting in translation of a fusion protein in any of the aforementioned host systems. For example, a chimeric DME protein containing a heterologous moiety that can be recognized by a commercially available antibody may facilitate the screening of peptide libraries for inhibitors of DME activity. Hleterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable imnunoaffinity purification of fusion proteins using commercially available monoclonal and polyclonal antibodies that specifically recognize these epitope tags. A fusion protein may also be engineered to contain a proteolytic cleavage site located between the DME encoding sequence and the heterologous protein sequence, so that DME maybe cleavedaway from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled DME may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled arino acid precursor, for example, ³⁵S-methionine.

DME of the present invention or fragments thereof may be used to screen for compounds that specifically bind to DME. At least one and up to a plurality of test compounds may be screened for specific binding to DME. Examples of test compounds include antibodies, oligonucleotides, proteins (e.g., receptors), or small molecules.

In one embodiment, the compound thus identified is closely related to the natural ligand of DME, e.g., a ligand or fragment thereof, a natural substrate, a structural or functional niimetic, or a naturalbinding partner. (See, e.g., Coligan, J. E. et al. (1991) Current Protocols in Immunology 1(2): Chapter 5.) Similarly, the compound can be closely related to the natural receptor to which DME binds, or to at least a fragment of the receptor, e.g., the ligand binding site. In either case, the compound can be rationally designed using known techniques. In one embodiment, screening for these compounds involves producing appropriate cells which express DME, either as a secreted protein or on the cell membrane. Preferred cells include cells from mammals, yeast, Drosophila, or E. coli. Cells expressing DME or cell membrane fractions which contain DME are then contacted with a test compound and binding, stimulation, or inhibition of activity of either DME or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with DME, either in solution or affixed to a solid support, and detecting the binding of DME to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

DME of the present invention or fragments thereof may be used to screen for compounds that modulate the activity of DME. Such compounds may include agonists, antagonists, or partial or inverse agonists. In one embodiment, an assay is performed under conditions permissive for DME activity, wherein DME is combined with at least one test compound, and the activity of DME in the presence of a test compound is compared with the activity of DME in the absence of the test compound. A change in the activity of DME in the presence of the test compound is indicative of a compound that modulates the activity of DME. Alternatively, a test compound is combined with an in vitro or cell-free system comprising DME under conditions suitable for DME activity, and the assay is performed. In either of these assays, a test compound which modulates the activity of DME may do so indirectly and need not come in direct contact with the test compound. At least one and up to a plurality of test compounds may be screened.

In another embodiment, polynucleotides encoding DME or their mammalian homologs may be “knocked out” in an animal model system using homologous recombination in embryonic stem (ES) cells. Such techniques are well known in the art and are useful for the generation of animal models of human disease. (See, e.g., U.S. Pat. Nos. 5,175,383 and 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination Alternatively, homologous recombination takes place using the Cre-loxP system to knockout a gene of interest in a tissue- or developmental stage-specific manner (Marth, J. D. (1996) Clin. Invest 97:1999-2002; Wagner, K. U. et al. (1997) Nucleic Acids Res. 25:43234330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding DME may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding DME can also be used to create “knockin” humanized animals (pigs) or transgenic animals (mice or rats) to model human disease. With knockin technology, a region of a polynucleotide encoding DME is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress DME, e.g., by secreting DME in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of DME and drug metabolizing enzymes. In addition, the expression of DME is closely associated with a variety of diseased tissues, including that of the brain, prostate, bone, intestine, and breast. Therefore, DME appears to play a role in autoimmunefmflammatory, cell proliferative, developmental, endocrine, eye, metabolic, and gastrointestinal disorders, including liver disorders. In the treatment of disorders associated with increased DME expression or activity, it is desirable to decrease the expression or activity of DME. In the treatment of disorders associated with decreased DME expression or activity, it is desirable to increase the expression or activity of DME.

Therefore, in one embodiment, DME or a fragment or derivative thereof maybe administered to a subject to treat or prevent a disorder associated with decreased expression or activity of DME. Examples of such disorders include, but are not limited to, an autoimmune/inflammatory disorder, such as acquired immunodeficiency syndrome (AIDS), Addison's disease, adult respiratory distress syndrome, allergies, ankylosing spondylitis, amyloidosis, anemia, asthma, atherosclerosis, autoirumune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erytlroblastosis fetalis, erythema n dosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjdgren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a cell proliferative disorder, such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a developmental disorder, such as renal tubular acidosis, anemia, Cusbcing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Matie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; an endocrine disorder, such as disorders of the hypothalamus and pituitary resulting from lesions such as primary brain tumors, adenomas, infarction associated with pregnancy, hypophysectomy, aneurysms, vascular malformations, thrombosis, infections, immunological disorders, and complications due to head trauma; disorders associated with hypopituitarism including hypogonadism, Sheehan syndrome, diabetes insipidus, Kallman's disease, Hand-Schuller-Christian disease, Letterer-Siwe disease, sarcoidosis, empty serla syndrome, and dwarfism; disorders associated with hyperpituitarism including acromegaly, giantism, and syndrome of inappropriate antidiuretic hormone (ADH) secretion (SIADH) often caused by benign adenoma; disorders associated with hypothyroidism including goiter, myxedema, acute thyroiditis associated with bacterial infection, subacute thyroiditis associated with viral infection, autoimmune thyroiditis (Hashimoto's disease), and cretinism; disorders associated with hyperthyroidism including thyrotoxicosis and its various formns, Grave's disease, pretibial myxedema, toxic multinodular goiter, thyroid carcinoma, and Plummer's disease; disorders associated with hyperparathyroidism including Corn disease (chronic hypercalemia); pancreatic disorders such as Type I or Type II diabetes mellitus and associated complications; disorders associated with the adrenals such as hyperplasia, carcinoma, or adenoma of the adrenal cortex, hypertension associated with alkalosis, amyloidosis, hypokalemia, Cushing's disease, Liddle's syndrome, and Amnold-Healy-Gordon syndrome, pheochromocytoma tumors, and Addison's disease; disorders associated with gonadal steroid hormones such as: in women, abnormal prolactin production, infertility, endometriosis, perturbations of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis; and, in men, Leydig cell deficiency, male climacteric phase, and germinal cell aplasia, hypergonadal disorders associated with Leydig cell tumors, androgen resistance associated with absence of androgen receptors, syndrome of 5 α-reductase, and gynecomastia; an eye disorder, such as conjunctivitis, keratoconjunctivitis sicca, keratitis, episcleritis, iritis, posterior uveitis, glaucoma, amaurosis fugax, ischemic optic neuropathy, optic neuritis, Leber's hereditary optic neuropathy, toxic optic neuropathy, vitreous detachment, retinal detachment, cataract, macular degeneration, central serous chorioretinopathy, retinitis pigmentosa, melanoma of the choroid, retrobulbar tumor, and chiasmal tumor; a metabolic disorder, such as Addison's disease, cerebrotendinous xanthomatosis, congenital adrenal hyperplasia, coumarin resistance, cystic fibrosis, diabetes, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperdtyroidism, hypoglycemia, hypothyroidism, hyperlipidemia, hyperlipemia, lipid myopathies, lipodystrophies, lysosomal storage diseases, Menkes syndrome, occipital horn syndrome, mannosidosis, neuraminidase deficiency, obesity, pentosuria phenylketonuria, pseudovitamin D-deficiency rickets; and a gastrointestinal disorder, such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, hereditary hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointetinal hemorrhage, acquired immunodeficiency syndrome (AIDS) enteropathy, jaundice, hepatic encephalopathy, hepatorenal syndrome, hepatic steatosis, hemochromatosis, arlson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas.

In another embodiment, a vector capable of expressing DME or a fragment or derivative thereof may be administered to a subject to treat or prevent a disorder associated with decreased expression or activity of DME including, but not limited to, those descnbed above.

In a further embodiment, a composition comprising a substantially purified DME in conjunction with a suitable pharmaceutical carrier may be administered to a subject to treat or prevent a disorder associated with decreased expression or activity of DME including, but not limited to, those provided above.

In still another embodiment, an agonist which modulates the activity of DME may be administered to a subject to treat or prevent a disorder associated with decreased expression or activity of DME including, but not limited to, those listed above.

In a further embodiment, an antagonist of DME may be administered to a subject to treat or prevent a disorder associated with increased expression or activity of DME. Examples of such disorders include, but are not limited to, those autoirnmunefinflarnmatory, cell proliferative, developmental, endocrine, eye, metabolic, and gastrointestinal disorders, including liver disorders described above. In one aspect, an antibody which specifically binds DME may be used directly as an antagonist or indirectly as a targeting or delivery mechanism for bringing a pharmaceutical agent to cells or tissues which express DME.

In an additional embodiment, a vector expressing the complement of the polynucleotide. encoding DME may be administered to a subject to treat or prevent a disorder associated with increased expression or activity of DME including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of DME may be produced using methods which are generally known in the arL In particular, purified DME may be used to produce antibodies or to screen libraries of pharmaceutical agents to identify those which specifically bind DME. Antibodies to DMB may also be generated using methods that are well known in the art. Such antibodies may include, but are not limited to, polyclonal, monoclonal chimeric, and single chain antibodies, Fab fragments, and fragments produced by a Fab expression library. Neutralizing antibodies (i.e., those which inhibit dimer formation) are generally preferred for therapeutic use.

For the production of antibodies, various hosts including goats, rabbits, rats, mice, humans, and others may be immunized by injection with DME or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLI, and dinitrophenol. Among adjuvants used in humans, BCG (bacilh Calmette-Guerin) and Corynebacterium parvum are especially preferable.

It is preferred that the oligopeptides, peptides, or fragments used to induce antibodies to DME have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of DME amino acids may be fused with those of another protein, such as KIM, and antibodies to the chimeric molecule may be produced.

Monoclonal antibodies to DME maybe prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:3142; Cote, R. J. et al. (1983) Proc. Natl. Acad. Sci. USA 80:2026-2030; and Cole, S.P. et al. (1984) Mol. Cell Biol. 62:109-120.) [symbol] D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for DME may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries maybe constructed to allow rapid and easy identification of monoclonal Fab fragments with the desired specificity. (See, e.g., Huse, W. D. et al. (1989) Science 246:1275-1281.)

Various immunoassays maybe used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between DME and its specific antibody. A two-site, monoclonal-based immunoassay utilizing monoclonal antibodies reactive to two non-interfering DME epitopes is generally used, but a competitive binding assay may also be employed (Pound, supra).

Various methods such as Scatchard analysis in conjunction with radioimmunoassay techniques may be used to assess the affinity of antibodies for DME. Affnity is expressed as an association constant, Kₐ, which is defined as the molar concentration of DME-antibody complex divided by the molar concentrations of free antigen and free antibody under equilibrium conditions. The Kₐ determined for a preparation of polyclonal antibodies, which are heterogeneous in their affinities for multiple DME epitopes, represents the average affinity, or avidity, of the antibodies for DME. The Kₐ determined for a preparation of monoclonal antibodies, which are monospecific for a particular DME epitope, represents a true measure of affinity. High-affinity antibody preparations with Kₐ ranging from about 10⁹ to 10¹² L/mole are preferred for use in immunoassays in which the DME-antibody complex must withstand rigorous manipulations. Low-affinity antibody preparations with Kₐ ranging from about 10⁶ to 10⁷ L/mole are preferred for use in immunopurification and similar procedures which ultimately require dissociation of DME, preferably in active form, from the antibody (Catty, D. (1988) Antibodies. Volume I: A Practical Approach, IRL Press, Washington D.C.; liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/nl, is generally employed in procedures requiring precipitation of DME-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, the polynucleotides encoding DME, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding DME. Such technology is well known in the art, and antisense oligonucleotides or larger fragments can be designed from various locations along the coding or control regions of sequences encoding DME. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E. et al. (1998) J. Allergy Clin. ltrunol. 102(3):469-475; and Scanlon, K. J. et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Phariacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding DME may be used for somatic or germline gene therapy. Gene therapy may be performed to (i) correct a genetic deficiency (e.g., in the cases of severe combined immunodeficiency (SCID)X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal R. G. et al. (1995) Hum. Gene Therapy 6:667-703), thalassamias, familial hypercholesterolemia, and hemophilia resulting from Factor VIII or Factor IX deficiencies (Crystal, R.G. (1995) Science 270:404-410; Verma, IM. and N. Sonia (1997) Nature 389:239-242)), (il) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA. 93:11395-11399), hepatitis B or C virus (HBV, HCV); fungal parasites, such as Candida albicans and Paracoccidioides brasiliensis; and protozoan parasites such as Plasmodium falcinarum and Tryanosoma cruzi). In the case where a genetic deficiency in DME expression or regulation causes disease, the expression of DME from an appropriate population of transduced cells may alleviate the clinical manifestations caused by the genetic deficiency.

In a further embodiment of the invention, diseases or disorders caused by deficiencies in DME are treated by constructing mammalian expression vectors encoding DME and introducing these vectors by mechanical means into DME-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J -L. and H. Récipon (1998) Curr. Opin. Biotechnol. 9:445-450).

Expression vectors that may be effective for the expression of DME include, but are not limited to, the PcDNA 3.1, EP1TAG, PRCCMV2, PREP, PVAX, PCR2-TOPOTA vectors (Invitrogen, Carlsbad Calif.), PCMV-SCRRIFT, PCMV-TAG, PEGSHIPERV (Stratagene, La Jolla Calif.), and PTET-OFF, PTEJT-ON, PTRE2, PTRE2-LUC, PITK-HYG (Clontech, Palo Alto Calif.). DME may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol 9:451456), commercially available in the T-REX plasmid nitrogen)); the ecdysone-inducible promoter (available in the plasmids PVGRXR and PIND; Invitrogen); the FK506/rapamycin inducible promoter; or the RU486/mifepristone inducible promoter (Rossi F. M. V. and Blau, H. M. supra)), or (iii) a tissue-specific promoter or the native promoter of the endogenous gene encoding DME from a normal individual.

Commercially available liposome transformation kits (e.g., the PERFECT LIPID TRANSFECMUON KIT, available from Invitrogen) aflow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized manmalian transfection protocols.

In another embodiment of the invention, diseases or disorders caused by genetic defects with respect to DME expression are treated by constructing a retrovirus vector consisting of (i) the polynucleotide encoding DME under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol 61:1639-1646; Adam, M. A. and A. D. Miller (1988) J. Virol. 62:3802-3806; Dull, Tₘ et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ Tells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In the alternative, an adenovirus-based gene therapy delivery system is used to deliver polynucleotides encoding DME to cells which have one or more genetic abnormalities with respect to the expression of DME. The construction and packaging of adenovirus-based vectors are well known to those with ordinary skill in the arl. Replication defective adenovirns vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another alternative, a herpes-based, gene therapy delivery system is used to deliver polynucleotides encoding DME to target cells which have one or more genetic abnormalities with respect to the expression of DME. The use of herpes simplex virus (HSV)-based vectors may be especially valuable for introducing DME to cells of the central nervous system, for which HSV has a tropism. The construction and packaging of herpes-based vectors are well known to those with ordinary skill in the art. A replication-competent herpes simnplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the controlof the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasrnids containing different segments of the large herpesvirus genomes, the growth and propagation of herpesvirus, and the infection of cells with herpesvinis are techniques well known to those of ordinary skill in the art.

In another alternative, an alphavirus (positive, single-stranded RNA vinis) vector is used to deliver polynucleotides encoding DME to target cells. The biology of the prototypic alphavirus, Semliki Forest Virus (SFV), has been studied extensively and gene transfer vectors have been based on the SFV genome (Garoff, H. and K. -J. Li (1998) Curr. Opin. Biotechnol 9:464-469). During alphavirus RNA replication, a subgenomic RNA is generated that normally encodes the viral capsid proteins. This subgenomic RNA replicates to higher levels than the fill length genoric RNA, resulting in the overproduction of capsid proteins relative to the viral proteins with enzymatic activity (e.g., protease and polymerase). Similarly, inserting the coding sequence for DME into the alphavirus genome in place of the capsid-coding region results in the production of a large number of DME-coding RNAs and the synthesis of high levels of DME in vector transduced cells. While alphavirus infection is typically associated with cell lysis within a few days, the ability to establish a persistent infection in hamster normal kidney cells BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of DMB into a variety of cell types. The specific transduction of a subset of cells in a S population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been descnbed in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of niRNA by preventing the transcript from binding to nbosomes.

Ribozymes, enzymatic RNA molecules, may also be used to catalyze the specific cleavage of RNA. The mechanism of nrbozyme action involves sequence-specific hybridization of the ribozyme molecule to complementary target RNA, followed by endonucleolytic cleavage. For example, engineered hammerhead motif nbozyme molecules may specifically and efficiently catalyze endonucleolytic cleavage of sequences encoding DME.

Specific ribozyme cleavage sites within any potential RNA target are initially identified by scanning the target molecule for nrbozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and nbozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding DME. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding DUE. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either irhibitors or promoters of polynucleotide expression. Thus, in the treatment of disorders associated with increased DME expression or activity, a compound which specifically inhibits expression of the polynucleotide encoding DME may be therapeutically useful, and in the treatment of disorders associated with decreased DME expression or activity, a compound which specifically promotes expression of the polynucleotide encoding DUE may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding DME is exposed to at least one test compound thus obtained. The sample may comprise, for example, an intact or permeabilized cell, or an in vitro cell-free or reconstituted biochemical system Alterations in the expression of a polynucleotide encoding DME are assayed by any method commonly known in the art. Typically, the expression of a specific nucleotide is detected by hybridization with a probe having a nucleotide sequence complementary to the sequence of the polynucleotide encoding DME. The amount of hybridization maybe quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, nbonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the arl. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient Excipients may include, for example, sugars, starches, celluloses, gums, and proteins. Various formulations are commonly known and are thoroughly discussed in the latest edition of Remington's Eharmaceutical Sciences (Maack Publishing, Easton Pa.). Such compositions may consist of DME, antibodies to DME, and rnimetics, agonists, antagonists, or inhibitors of DME.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical,. sublingual, or rectal means.

Compositions for puhlonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising DME or fragments thereof. For example, liposome preparations containing a cell-impermeable macromolecule may promote cell fusion and intracellular delivery of the macromolecule. Alternatively, DME or a fragment thereof may be joined to a short cationic N-terminal portion from the IRV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example DME or fragments thereof, antibodies of DME, and agonists, antagonists or inhibitors of DME, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the s severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind DME may be used for the diagnosis of disorders characterized by expression of DME, or in assays to monitor patients being treated with DME oragonists, antagonists, or inhibitors of DME. Antibodies useful for diagnostic purposes may be prepared in the same manner as described above for therapeutics. Diagnostic assays for DME include methods which utilize the antibody and a label to detect DME in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring DME, including EULSAs, RIAs, and FACS, are known in the art and provide a basis for diagnosing altered or abnormal levels of DME expression. Normal or standard values for DME expression are established by combining body fluids or cell extracts taken from normal mammalian subjects, for example, human subjects, with antibodies to DME under conditions suitable for complex formation. The amount of standard complex formation may be quantitated by various methods, such as photometric means. Quantities of DME expressed in subject, control, and disease samples from biopsied tissues are compared with the standard values. Deviation between standard and subject values establishes the parameters for diagnosing disease.

In another embodiment of the invention, the polynucleotides encoding DME may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of DMEB maybe correlated with disease. The diagnostic assay may be used to determine absence, presence, and excess expression of DME, and to monitor regulation of DME levels during therapeutic intervention.

In one aspect, hybridization with PCR probes which are capable of detecting polynucleotide sequences, including genomic sequences, encoding DMEE or closely related molecules maybe used to identify nucleic acid sequences which encode DME. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding DME, allelic variants, or related sequences.

Probes may also be used for the detection of related sequences, and may have at least 50% sequence identity to any of the DME encoding sequences. The hybridization probes of the subject invention may be DNA or RNA and may be derived from the sequence of SEQ ID NO:20-38 or from genomic sequences including promoters, enhancers, and introns of the DME gene.

Means for producing specific hybridization probes for DNAs encoding DME include the cloning of polynucleotide sequences encoding DME or DME derivatives into vectors for the production of niRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidinibiotin coupling systems, and the like.

Polynucleotide sequences encoding DME may be used for the diagnosis of disorders associated with expression of DMEE. Examples of such disorders include, but are not limited to, an autoimmune/inflammatory disorder, such as acquired immunodeficiency syndrome (AIDS), Addison's disease, adult respiratory distress syndrome, allergies, ankylosing spondylitis, amyloidosis, anemia, asthrma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashirnoto's thyroiditis, hypereosinopbilia, irritable bowel syndrome, multiple sclerosis, myasthenia gravis, myocardial or pericardial inflammation, osteoardhritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoan and hehninthic infections, and trauma; a cell proliferative disorder, such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a developmental disorder, such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; an endocrine disorder, such as disorders of the hypothalamus and pituitary resulting fiom lesions such as primary brain tumors, adenomas, infarction associated with pregnancy, hypophysectomy, aneurysms, vascular malformations, thrombosis, infections, immunological disorders, and complications due to head trauma; disorders associated with hypopituitarism including hypogonadism, Sheehan syndrome, diabetes insipidus, Kalman's disease, Hand-Schuller-Christian disease, Letterer-Siwe disease, sarcoidosis, empty sella syndrome, and dwarfism; disorders associated with hyperpituitarism including acromegaly, giantism, and syndrome of inappropriate antidiuretic hormone (ADI) secretion (SIADH) often caused by benign adenoma; disorders associated with hypothyroidism including goiter, myxedema, acute thyroiditis associated with bacterial infection, subacute thyroiditis associated with viral infection, autoimrnune thyroiditis (Hashimoto's disease), and cretinism; disorders associated with hyperthyroidism including thyrotoxicosis and its various forms, Grave's disease, pretibialmyxedema, toxic multinodular goiter, thyroid carcinoma, and Plummer's disease; disorders associated with hyperparathyroidism including Conn disease (chronic hypercalemia); pancreatic disorders such as Type I or Type II diabetes mellitus and associated complications; disorders associated with the adrenals such as hyperplasia, carcinoma, or adenoma of the adrenal cortex, hypertension associated with alkalosis, amyloidosis, hypokalemia, Cushing's disease, Liddle's syndrome, and Arnold-Healy-Gordon syndrome, pheochromocytoma tumors, and Addison's disease; disorders associated with gonadal steroid hormones such as: in women, abnormal prolactin production, infertility, endometriosis, perturbations of the menstrual cycle, polycystic ovarian disease, hyperprolactinemia, isolated gonadotropin deficiency, amenorrhea, galactorrhea, hermaphroditism, hirsutism and virilization, breast cancer, and, in post-menopausal women, osteoporosis; and, in men, Leydig cell deficiency, male climacteric phase, and germinal cell aplasia, hypergonadal disorders associated with Leydig cell tumors, androgen resistance associated with absence of androgen receptors, syndrome of 5 α-reductase, and gynecomastia; an eye disorder, such as conjunctivitis, keratoconjunctivitis sicca, keratitis, episcleritis, iritis, posterior uveitis, glaucoma, amaurosis fagax, ischemic optic neuropathy, optic neuritis, Leber's hereditary optic neuropathy, toxic optic neuropathy, vitreous detachment, retinal detachment, cataract, macular degeneration, central serous chorioretinopathy, retinitis pigmentosa, melanoma of the choroid, retrobulbar tumor, and chiasmal tumor; a metabolic disorder, such as Addison's disease, cerebrotendinous xanthomatosis, congenital adrenal hyperplasia, coumarin resistance, cystic fibrosis, diabetes, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, Menkes syndrome, occipital horn syndrome, mannosidosis, neuraminidase deficiency, obesity, pentosuria phenylketonuria, pseudovitamin D-deficiency rickets; and a gastrointestinal disorder, such as dysphagia, peptic esophagitis, esophageal spasm, esophageal strictrre, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, hereditary hyperbilimbinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha,-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas. The polynucleotide sequences encoding DMB may be used in Southern or northern analysis, dot blot, or other membrane-based technologies; in PCR technologies; in dipstick, pin, and multiformat ELISA-like assays; and in microarrays utilizing fluids or tissues from patients to detect altered DME expression. Such qualitative or quantitative methods are well known in the art.

In a particular aspect, the nucleotide sequences encoding DME may be useful in assays that detect the presence of associated disorders, particularly those mentioned above. The nucleotide sequences encoding DME may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding DMB in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of DME, a normal or standard profile for expression is established. This may be accomplished by combining body fluids or cell extracts taken from normal subjects, either animal or human, with a sequence, or a fragment thereof, encoding DME, under conditions suitable for hybridization or amplification. Standard hybridization may be quantified by comparing the values obtained from normal subjects with, values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) inbiopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding DME may involve the use of PCR. These oligomers may be chemically synthesized, generated enzymatically, or produced in vitro. Oligomers will preferably contain a fragment of a polynucleotide encoding DME, or a fragment of a polynucleotide complementary to the polynucleotide encoding DME, and will be employed under optimized conditions for identification of a specific gene or condition. Oligomers may also be employed under less stringent conditions for detection or quantification of closely related DNA or RNA sequences.

In a particular aspect, oligonucleotide primers derived from the polynucleotide sequences encoding DME may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (FSSCP) methods. In SSCP, oligonucleotide primers derived from the polynucleotide sequences encoding DME are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

Methods which may also be used to quantify the expression of DME include radiolabeling or biotinylating nucleotides, coamphfication of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotide sequences described herein may be used as elements on a microarray. The microarray can be used in transcript imaging techniques which monitor the relative expression levels of large numbers of genes simultaneously as descnbed below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, DME, fragments of DME, or antibodies specific for DME may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifyg the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occcrring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nibgov/oc/news/toxchip.htm.) Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention maybe quantified. The transcript levels in the treated biological sample are compared with levels in an untreated biological sample. Differences in the transcript levels between the two samples are indicative of a toxic response caused by the test compound in the treated sample.

Another particular embodiment relates to the use of the polypeptide sequences of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifyig the number of expressed proteins and their relative abundance under given conditions and at a given time. A profile of a cell's proteome may thus be generated by separating and analyzing the polypeptides of a particular tissue or cell type. In one embodiment, the separation is achieved using two dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, sura). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomic profile may also be generated using antibodies specific for DME to quantify the levels of DME expression. In one embodiment, the antibodies are used as elements on a microarray, and protein expression levels are quantified by exposing the microarray to the sample and detecting the levels of protein bound to each array element CLueking, A. et al. (1999) Anal. Biochenm 270:103-111; Mendoze, L. G. et al (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recdgnized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London, hereby expressly incorporated by reference.

In another embodiment of the invention, nucleic acid sequences encoding DME may be used to generate hybridization probes useful in mapping the. naturally occurring genornic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat Genet. 15:345-355; Price, C. M. (1993) Blood Rev. 7:127-134; and Trask, B. J. (1991) Trends Genet. 7:149-154.) Once mapped, the nucleic acid sequences of the invention maybe used to develop genetic linkage maps, for example, which correlate the inheritance of a disease state with the inheritance of a particular chromosome region or restriction fragment length polymorphism (RJW). (See, for example, Lander, E. S. and D. Botstein (1986) Proc. And. Acad. Sci. USA 83:7353-7357.)

Fluorescent in situ hybridization (DISH) may be correlated with other physical and genetic map data. (See, e.g., Heinz-llrich, et al. (1995) in Meyers, supra, pp. 965-968.) Examples of genetic map data can be found in various scientific journals or at the Online Mendelian lhzeritance in Man (OMIM) World Wide Web site. Correlation between the location of the gene encoding DMB on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 1 lq22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, DME, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between DME and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with DME, or fragments thereof, and washed. Bound DME is then detected by methods well known in the art. Purified DME can also be coated directly onto plates for use in the aforementioned drug screening techniques. Alternatively, non-neutralizing antibodies can be used to capture the peptide and immobilize it on a solid support.

In another embodirnem, one may use competitive drug screening assays in which neutralizing antibodies capable of binding DME specifically compete with a test compound for binding DME. In this manner, antibodies can be used to detect the presence of any peptide which shares one or more antigenic determinants with DME.

In additional embodiments, the nucleotide sequences which encode DME may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications and publications, mentioned above and below, incuding U.S. Ser. Nos. 60/223,055, 60/224,728, 60/226,440, 60/228,067, 60/230,063, 60/232,244, and 60/234,269, are expressly incorporated by reference herein.

### EXAMPLES

I. Construction of cDNA Libraries

Incyte cDNAs were derived from cDNA libraries described in the LIESEQ GOLD database (Incyte Genonucs, Palo Alto Calif.) and shown in Table 4, column 5. Some tissues were homogenized and lysed in guanidinium isothiocyanate, while others were homogenized and lysed in phenol or in a suitable mixture of denaturants, such as TRIZOl. (Life Technologies), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX MnRNA purification kit (QIAGEN). Alternatively, RNA was isolated directly from tissue lysates using other RNA isolation kits, e.g., the POLY(A)PURE MnRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.1-6.6.) Reverse transcription was initiated using oligo d( or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYl. S1000, SEPHAROSE CL2B, or SEPHAROSE CIAB column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasnid (Stratagene), PSPORT1 plasmid (Life Technologies), PcDNA2.1 plasnud (nvitrogen, Carlsbad Calif.), PBK-CMV plasmid (Stratagene), PCR2-TOPOTA (nvitrogen), or pINCY (Incyte Genomics, Palo Alto Calif.), or derivatives thereof. Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids obtained as described in Example 1 were recovered from host cells by in vivo excision using the UNIAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELl. 8 Plasmid, QIAWELl. 8 Plus Plasmid, QIAWELl. 8 Ultra Plasmid purification systems or the R.E.A.L. PREP 96 plasmid purification kit from QIAGEN. Following precipitation, plasmids were resuspended in 0.1 ml of distilled water and stored, with or without lyophifization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture; Samples were processed and stored in 384-well plates, and the concentration of amplified plasmid DNA was quantified fluorometrcally using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as descnbed in Example II were sequenced as follows. Sequencing reactions were processed using standard methods or high-throughput instrumentation such as the ABI CATALYST 800 (Applied Biosystems) thermal cycler or the PlC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the arl. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBaik primate, rodent, mammalian, vertebrate, and eukaryote databases, and BLOCKS, PRINTS, DOMO, PRODOM, and hidden Markov model (HMM)-based protein family databases such as PFAM. ( is a probabilistic approach which analyzes consensus primary structures of gene families. See, for example, Eddy, S. R. (1996) Curr. Opin. Struct. Biol. 6:361-365.) The queries were performed using programs based on BLAST, FASTA, BUMPS, and R. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the correspondingfull length polypeptide sequences. Alternatively, a polypeptide of the invention may begin at any of the methionine residues of the full length translated polypeptide. Full length polypeptide sequences were subsequently analyzed by querying against databases such as the GenBank protein databases (genpept), SwissProt, BLOCKS, PRINTS, DOMO, PRODOM, Prosite, and hidden Markov model (H)-based protein family databases such as PFAM. Pull length polynucleotide sequences are also analyzed using MAcDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold paratneters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ED NO:20-3 8. Fragments from about 20 to about 4000 nucleotides which are useful in hybridization and amplification technologies are described in Table 4, column 4.

IV. Identification and Editing of Coding Sequences from Genomic DNA

Putative drug metabolizing enzymes were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codon. The output of is Genscan is a FASTA database of polynucleotide and polypeptide sequences. The maximum range of sequence for Genscan to analyze at once was set to 30 kb. To determine which of these Genscan predicted cDNA sequences encode drug metabolizing enzymes, the encoded polypeptides were analyzed by querying against PFAM models for drug metabolizing enzymes. Potential drug metaboliziug enzymes were also identified by homology to Incyte cDNA sequences that had been annotated as drug metabolizig enzymes. TIhese selected Genscan-predicted sequences were then compared by BLAST analysis to the genpept and gbpri public databases. Where necessary, the Genscsm-predicted sequences were then edited by comparison to the top BLAST hit from genpept to correct errors in the sequence predicted by Gefscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genoinic Sequence Data with cDNA Sequence Data

“Stitched” Sequences

Partial cDNA sequences were extended with exons predicted by the Genscan gene identification program described in Example IV. Partial cDNAs assembled as descnbed in Example Im were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of DME Encoding Polynucleotides

The sequences which were used to assemble SEQ ID NO:20-38 were compared with sequences from the Incyte LIFESEQ database and public domain databases using BLAST and other implementations of the Smith-Waterman algorithm. Sequences from these databases that matched SEQ ID NO:20-38 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Genethon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although tvis can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http:/lwww.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LIMSEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotide sequences encoding DME are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflamation, neurological, trauma, cardiovascular, pooled, and other, and the number of libraries in each category is counted and divided by the total number of libraries across all categories. The resulting percentages reflect the tissue- and disease-specific expression of cDNA encoding DME. cDNA sequences and cDNA library/tissue information are found in the UFESEQ GOLD database (Incyte Genomics, Palo Alto Calif.).

VIII. Extension of DME Encoding Polynucleotides

Fall length polynucleotide sequences were also produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment The initial primers were designed using OLUGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5:Steps 2, 3, and 4 repeated 20 times; Step 6: 68° C., 5 min; Step 7:storage at 4° C. In the alternative, the parameters for primer pair T7 and SK+were as follows: Step 1: 94° C., 3 min; Step 2: 94° C., 15 sec; Step 3: 57° C., 1 min; Step 4: 68° C., 2 min; Step 5:Steps 2, 3, and 4 repeated 20 times; Step 6: 68° C., 5 min; Step 7:storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsindki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Pharmacia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersbam Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5:steps 2, 3, and 4 repeated 29 times; Step 6: 72° C. 5 min; Step 7:storage at 4° C. DNA was quantified by PICOGREEN reagent (Molecular Probes) as descnbed above. Samples with low DNA recoveries were reamplified using the same conditions as descnbed above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Applied Biosystems).

In like manner, full length polynucleotide sequences are verified using the above procedure or are used to obtain 5′ regulatory sequences using the above procedure along with oligonucleotides designed for such extension, and an appropriate genomic library.

IX. Labeling and Use of Individual Hybridization Probes

Hybridization probes derived from SEQ ID NO:20-38 are employed to screen cDNAs, genomic DNAs, or mRNAs. Although the labeling of oligonucleotides, consisting of about 20 base pairs, is. specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Pharmacia Biotech), and T4 polynucleotide kinase (DuPont NEN, Boston Mass.). The labeled oligonucleotides are substantially purified using a SEPHADEX G-25 superfine size exclusion dextran bead column (Amersham Pharmacia Biotech). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu I (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

X. Microarrays

The linkage or synthesis of array elements upon a microarray can be achieved utilizing photolithography, piezoelectric printing (mkjet printing, See, e.g., Baldeschweiler, supra), mechanical microspotting technologies, and derivatives thereof. The substrate in each of the aforementioned technologies should be uniform and solid with a non-porous surface (Schena (1999), suira). Suggested substrates include silicon, silica, glass slides, glass chips, and silicon wafers. Alternatively, a procedure analogous to a dot or slot blot may also be used to arrange and link elements to the surface of a substrate using thermal, UW, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21 mer), 1× first strand buffer, 0.03 units/μl RNase inhibitor, 500 μM dATP, 500 μM dGTP, 500 μM dTTP, 40 μM dCTIP, 40 μM dCTP-Cy3 (BDS) or dCTP-Cy5 (Amersham Pharmacia Biotech). The reverse transcription reaction is performed in a 25 ml volume containing 200 ng poly(A)⁺ RNA with GEMBRIGHT kits (hncyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a ST ATATR Uv-crosslikker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cys labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber contaiing the arrays is incubated for about 6.5 hours at 60° C. The arrays are washed for 10 min at 45OC in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Inova 70 mixed gas 10 W laser (Coherent, Inc., Santa Clara Calif.) capable of generating spectral lines at 488 num for excitation of Cy3 and at 632 nm for excitation of Cy5. The excitation laser light is S focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each florophore's emission spectrum.

A grid is superimposed over the fluorescence signal image such that the signal from each spot is centered in each element of the grid. The fluorescence signal within each element is then integrated to obtain a numerical value corresponding to the average intensity of the signal The software used for signal analysis is the GEMTOOLS gene expression analysis program (Incyte).

XI. Complementary Polynucleotides

Sequences complementary to the DME-encoding sequences, or any parts thereof, are used to detect, decrease, or inhibit expression of naturally occurring DME. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of DME. To inhibit transcription, a complementary oligonucleotide is designed from the most unique 5′ sequence and used to prevent promoter binding to the coding sequence. To inhibit translation, a complementary oligonucleotide is designed to prevent nbosomal binding to the DUE-encoding transcript

XII. Expression of DME

Expression and purification of DME is achieved using bacterial or virus-based expression systems. For expression of DME in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T′ bacteriophage promoter in conjunction with the lac operator regulatory element. Recombinant vectors are transformed into suitable bacterial hosts, e.g., BL21(DE3). Antibiotic resistant bacteria express DME upon induction with isopropyl beta-D-thiogalactopyranoside (DOG). Expression of DME in eukaryotic cells is achieved by infecting insect or manmmalian cell lines with recombinant Autographica californica nuclear polyhedrosis virus (AcMNPV), commonly known as baculovirus. The nonessential polyhedrin gene of baculovirus is replaced with cDNA encoding DME by either homologous recombination or bacterial-mediated transposition involving transfer plasniid intermediates. Viral infectivity is maintained and the strong polyhedrin promoter drives high levels of cDNA transcription. Recombinant baculovirus is used to infect Spodoptera frugiperda (Sf9) insect cells inmost cases, orhumanhepatocytes, insome cases. Infection of the latter requires additional genetic modifications to baculovirus. (See Engelhard, E. K. et al. (1994) Proc. Natl Acad. Sci. USA 91:3224-3227; Sandig, V. et al. (1996) Hum. Gene Ther. 7:1937-1945.)

In most expression systems, DME is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-lilodalton enzyme from Schistosoma japonicum, enables the purification of fusion proteins on immobilzed glutatluone under conditions that maintain protein activity and antigenicity (Amersham Pharmacia Biotech). Following purification, the GST moiety can be proteolytically cleaved Erom DME at specifically engineered sites. FLAG, an 8-amino acid peptide, enables irnmunoaffinity purification using commercially available monoclonal and polyclonal anti-FLAG antibodies (Eastman Kodak). 6-His. a stretch of six consecutive histidine residues, enables purification on metal-chelate resins (QIAGEN). Methods for protein expression and purification are discussed in Ausubel (1995, supra, ch. 10 and 16). Purified DME obtained by these methods can be used directly in the assays shown in Examples XVI, XVII, and XVIII, where applicable.

XIII. Functional Assays

DME function is assessed by expressing the sequences encoding DME at physiologically elevated levels in mammalian cell culture systems. cDNA is subcloned into a mammalian expression vector containing a strong promoter that drives high levels of cDNA expression. Vectors of choice include PCMV SPORT (Life Technologies) and PCR3.1 (Invitrogen, Carlsbad Calif.), both of which contain the cytomegalovirus promoter. 5-10 μg of recombinant vector are transiently transfected into a human cell line, for example, an endothelial or hematopojetic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of.choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometr, Oxford, New York N.Y.

The influence of DME on gene expression can be assessed using higbly purified populations of cells transfected with sequences encoding DME and either CD64 or CD64-GFP. CD64 and CD64-GFP are expressed on the surface of transfected cells and bind to conserved regions of human inuunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding DME and other genes of interest can be analyzed by northern analysis or microarray techniques.

XIV. Production of DME Specific Antibodies

DME substantially purified using polyacrylamide gel electrophoresis (PAGE; see, e.g., Harrington, M. G. (1990) Methods Enzymol. 182:488-495), or other purification techniques, is used to immunize rabbits and to produce antibodies using standard protocols.

Alternatively, the DME amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-termninus or in hydrophilic regions are well described in the art. (See, e.g., Ausubel, 1995, supra, ch. 11.)

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-DME activity by, for example, binding the peptide or DME to a substrate, blocking with 1% BSA, reacting with rabbit antisera, washing, and reacting with radio-iodinated goat anti-rabbit IgG.

XV. Purification of Naturally Occurring DME Using Specific Antibodies

Naturally occurring or recombinant DME is substantially purified by immunoaffuity chromatography using antibodies specific for DME. An immunoaffinity column is constructed by covalently coupling anti-DME antibody to an activated chromatographic resin, such as CNBr-activated SEPHAROSE (Amersham Pharmacia Biotech). After the coupling, the resin is blocked and washed according to the manufacturer's insructions.

Media containing DMB are passed over the immunoaffmity column, and the column is washed under conditions that allow the preferential absorbance of DME (e.g., high ionic strength buffers in the presence of detergent). The column is eluted under conditions that disrupt antibody/DME binding (e.g., a buffer of pH 2 to pH 3, or a high concentration of a chaotrope, such as urea or thiocyanate ion), and DME is collected.

XVI. Identification of Molecules Which Interact with DME

DME, or biologically active fragments thereof, are labeled with I. Bolton-Hunter reagent. (See, e.g., Bolton A. E. and W. M. Hunter (1973) Biochem. J. 133:529-539.) Candidate molecules previously arrayed in the wells of a multi-well plate are incubated with the labeled DME, washed, and any wells with labeled DME complex are assayed. Data obtained using different concentrations of DME are used to calculate values for the number, affinity, and association of DME with the candidate molecules.

Alternatively, molecules interacting with DME are analyzed using the yeast two-hybrid system as descnbed in Fields, S. and 0. Song (1989) Nature 340.245-246, or using commercially available kits based on the two-hybrid system, such as the MATCHMAKER system (Clontech).

DME may also be used in the PATHCATING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVII. Demonstration of DME Activity

Cytochrome P450 activity of DME is measured using the 4-hydroxylation of aniline. Aniline is converted to 4-aminophenol by the enzyme, and has an absorption maximum at 630 nm (Gibson and Skett, supra). This assay is a convenient measure, but underestimates the total hydroxylation, which also occurs at the 2- and 3- positions. Assays are performed at 37° C. and contain an aliquot of the enzyme and a suitable amount of aniline (approximately 2 mM) in reaction buffer. For this reaction, the buffer must contain NADPH or an NADPH-generating cofactor system. One formulation for this reactionbuffer includes 85 mM Tris pH 7.4, 15 mM MgCl₂, 50 mM nicotinamide, 40 mg trisodium isocitrate, and 2 units isocitrate dehydrogenase, with 8 mg NADP⁺ added to a 10 ml. reaction buffer stock just prior to assay. Reactions are carried out in an optical cuvette, and the absorbance at 630 nm is measured. The rate of increase in absorbance is proportional to the enzyme activity in the assay. A standard curve can be constructed using known concentrations of 4-aminophenoL 1α,25-dihydroxyvitamin D 24hydroxylase activity of DME is determined by monitoring the conversion of ³H-labeled 1α,25-dihydroxyvitamin D (1α,25(OH)₂D) to 24,25-dihydroxyvitamin D (24,25(OH)₂D) in transgenic rats expressing DME. 1 μg of 1α,25(0H)₂D dissolved in ethanol (or ethanol alone as a control) is administered intravenously to approximately 6week-old male transgenic rats expressing DME or otherwise identical control rats expressing either a defective variant of DME or not expressing DME. The rats are killed by decapitation after 8 hrs, and the kidneys are rapidly removed, rinsed, and homogenieed in 9 volumes of ice-cold buffer (15 mM Tris-acetate (pH 7.4), 0.19 M sucrose, 2 mM magnesium acetate, and 5 mM sodium succinate). A portion (e.g., 3 ml) of each homogenate is then incubated with 0.25 nM 1α,25(OH)₂[1-³H]D, with a specific activity of approximaately 3.5 GBq/mmol, for 15 minl at 37° C. under oxygen with constant shaking. Total lipids are extracted as described (Bligh, E. G. and W. J. Dyer (1959) Can. J. Biochem. Physiol. 37: 911-917) and the chloroform phase is analzd by BPLC using a FINEPAK SIl. column (JASCO, Tokyo, Japan) with a n-hexane,chloroform/methanol (10:2.5:1.5) solvent system at a flow rate of 1 ml/min. In the, alternative, the chloroform phase is analyzed by reverse phase BPLC using a J SPHERE ODS-AM column (YMC Co. Ltd., Kyoto, Japan) with an acetonitrile buffer system (40 to 100%, in water, in 30 min) at a flow rate of 1 min/min. The eluates are collected in fractions of 30 seconds (or less) and the amount of ³H present in each fraction is measured using a scintillation counter. By comparing the chrornatograms of control samples (i.e., samples comprising 1α,25-dfilydroxyvitamin D or 24,25-dihydroxyvitamin D (24,25(OH)₂D), with the chromatograms of the reaction products,te relative mobilities of the substrate (1α,25(OH)₂[1-³H]D) and product (24,25(OH)₂[1-³H]D) are determined and correlated with the fractions collected. The amount of 24,25(OH)₂[1-³H]D produced in control rats is subtracted from that of transgenic rats expressing DME. The difference in the production of 24,25(OH)₂[1-³H]D in the transgenic and control animals is proportional to the amount of 25-hydrolase activity of DNM present in the sample. Cornnrmation of the identity of the substrate and product(s) is conflimed by means of mass spectroscopy (Myamoto, Y. et al. (1997) J. Biol. Chem. 272:14115-14119).

Flavin-contain monooxygenase activity of DME is measured by chromatographic analysis of metabolic products. For example, Ring, B. J. et al. (1999; Drug Metab. Dis. 27:1099-1103) incubated IMO in 0. 1 M sodium phosphate buffer (pH 7.4 or 8.3) and 1 mM NADPH at 37° C., stopped the reaction with an organic solvent, and determined product formation by HPLC. Alternatively, activity is measured by monitoring oxygen uptak using a Clark-type electrode. For example, Ziegler, D. M. and Poulsen, L. L. (1978; Methods Eniymol. 52:142-151) incubated the enz::yme at 37° C. in an NADPH-generating cofactor system (similar to the one described above) contrining the substrate methimazole. The rate of oxygen uptake is proportional to enzye activity.

UDP glucuronyltransferase activity of DME is measured using a colorimetric determination of free amine groups (Gibson and Skett, sunra. An amine-containing substrate, such as 2-aminophenol, is incubated at 37° C. with an aliquot of the enzm in a reaction buffer contanig the necessary cofactors (40 mM Tris pH 8.0, 7.5 mM MgCl₂, 0.025% Triton X-100, 1 mM ascorbic acid, 0.75 mM UDP-glucuronic acid). After sufficient timne, the reaction is stopped by addition of ice-cold 20% trichloroacetic acid in 0. 1 M phosphate buffer pH 2.7, incubated on ice, and centiiged to clait the supemnatant Amy wunece 2-amrnophenol is destroyed in this step. Sufficient freshly-prepared sodium nitrite is then added; this step allows formation of the diazonium salt of the glucuronidated product. Excess nitrite is removed by addition of sufficient ammonium sulfamate, and the diazonium salt is reacted with an aromatic amine (for example, N-naphthylethylene diamine) to produce a colored azo compound which can be assayed spectrophotQmetrically (at 540 nm for the example). A standard curve can be constructed using known concentrations of aniline, which will form a chromophore with similar properties to 2-ainiophenol glucuronide.

Sulfotransferase activity of DME is measured using the incorporation of ³⁵S from [³⁵S]PAPS into a model substrate such as phenol (Folds, A. and Meek, J. L. (1973) Biochim. Biophys. Acta 327:365-374). An aliquot of enzyme is incubated at 37° C. with 1 ml. of 10 mM phosphate buffer pH 6.4, 50 μM phenol, 0.4-4.0 μM [³⁵S]PAPS. After sufficient time for 5-20% of the radiolabel to be transferred to the substrate, 0.2 mL. of 0.1 M barium acetate is added to precipitate protein and phosphate buffer. Then 0.2 mL. of 0.1 M Ba(OH)₂ is added, followed by 0.2 ml. ZnSO₄. The supernatant is cleared by centrifugation, which removes proteins as well as unreacted [³⁵S]PAPS. Radioactivity in the supernatant is measured by scintillation. The enzyme activity is determined from the number of moles of radioactivity in the reaction product.

Glutathione S-transferase activity of DME is measured using a model substrate, such as 2,4-dinitro-1-chlorobenzene, which reacts with glutathione to form a product, 2,4-dinitrophenyl-glutathione, that has an absorbance maximum at 340 nm. It is important to note that GSTs have differing substrate specificities, and the model substrate should be selected based on the substrate preferences of the GST of interest. Assays are performed at ambient temperature and contain an aliquot of the enzyme in a suitable reaction buffer (for example, 1 mM glutathione, 1 mM dinitrochlorobenzene, 90 mM potassium phosphate buffer pH 6.5). Reactions are carried out in an optical cuvette, and the absorbance at 340 nm is measured. The rate of increase in absorbance is proportional to the enzyme activity in the assay.

N-acyltransferase activity of DME is measured using radiolabeled amino acid substrates and measuring radiolabel incorporation into conjugated products. Enzyme is incubated in a reaction buffer containing an unlabeled acyl-CoA compound and radiolabeled amino acid, and the radiolabeled acyl-conjugates are separated from the unreacted amino acid by extraction into n-butanol or other appropriate organic solvent For example, Johnson, M. R. et al. (1990; J. Biol. Chem. 266:10227-10233) measured bile acidCoA:amino acid N-acyltransferase activity by incubating the enzyme with cholyl-CoA and ³H-glycine or ³H-taurine, separating the tritiated cholate conjugate by extraction into n-butanol, and measuring the radioactivity in the extracted product by scintillation Alternatively, N-acyltransferase activity is measured using the spectrophotometric determination of reduced CoA (CoASH) described below.

N-acetyltransferase activity of DME is measured using the transfer of radiolabel from [¹⁴C]acetyl-CoA to a substrate molecule (for example, see Deguchi, Tₘ (1975) J. Neurochem. 24:1083-5). Alternatively, a newer spectrophotometric assay based on DTNB (5,5′-dithio-bis(2-nitrobenzoic acid; Ellman's reagent) reaction with CoASH may be used. Free thiol-containing CoASH is formed during N-acetyltransferase catalyzed transfer of an acetyl group to a substrate. CoASH is detected using the absorbance of DTNB conjugate at 412 nm (De Angelis, J. et al. (1997) J. Biol. Chem. 273:3045-3050). Enzyme activity is proportional to therate of radioactivity incorporation into substrate, or the rate of absorbance increase in the spectrophotometric assay.

Protein arginine methyltransferase activity of DME is measured at 37° C. for various periods of time. S-adenosyl-L-[methyl-³H]methionine ([³H]AdoMet; specific activity=75 Ci/mmol; NEN Life Science Products) is used as the methyl-donor substrate. Useful methyl-accepting substrates include glutathione S-transferase fibrillarin glycine-arginine domain fusion protein (GST-GAR), heterogeneous nuclear rnbonucleoprotein (hnRNP), or hypomethylated proteins present in lysates from adenosine dialdehyde-treated cells. Methylation reactions are stopped by adding SDS-PAGE sample buffer. The products of the reactions are resolved by SDS-PAGE and visualized by fluorography. The presence, of ³H-labeled methyl-donor substrates is indicative of protein argimne methyltransferase activity of DME (Tang, J. et al. (2000) J. Biol. Chem. 275:7723-7730and Tang, J. et al. (2000) J. BioL Chem.275:19866-19876).

Aldo/keto reductase activity of DME is measured using the decrease in absorbance at 340 nm as NADPH is consumed. A standard reaction mixture is 135 mM sodium phosphate buffer (pH 6.2-7.2 depending on enzyme), 0.2 mM NADPH, 0.3 M lithium sulfate, 0.5-2.5 μg enzyme and an appropriate level of substrate. The reaction is incubated at 30° C. and the reaction is moritored continuously with a spectrophotometer. Enzyme activity is calculated as mol NADPH consumed/μg of enzyme.

Alcohol dehydrogenase activity of DME is measured using the increase in absorbance at 340 nm as NAD⁺ is reduced to NADH. A standard reaction mixture is 50 mM sodium phosphate, pH 7.5, and 0.25 mM EDTA. The reaction is incubated at 25° C. and monitored using a spectrophotometer. Enzyme activity is calculated as mol NADH produced/μg of enzyme.

DME activity is determined usmg methylurbelliferyl acetate as a substrate. The enzymatic reaction is initiated by adding approximately 10 μl of DME-contaiming sample to 1 ml of reaction buffer (90 mM KH₂PO₄, 40 mM KCl, pH 7.3) with 0.5 mM 4-methybilliferyl acetate at 37° C. The production of 4methylumbelliferone is monitored with a spectrophotomneter (ε₃₅₀=12.2 mM⁻¹ cm⁻¹) for 1.5 min Specific activity is expressed as micromoles of product formed per minute per milligram of protein and corresponds to the activity of DME in the sanmple (Evgenia, V. et al. (1997) J. Biol. Chein 272:14769-14775).

In the alternative, the cocaine benzoyl ester hydrolase activity of DME is measured by incubating approximately 0.1 ml of enyyme 3.3 mM cocaine in reaction buffer (50 mM NaH₂PO₄, pH 7.4) with 1 mM benzamidine, 1 mM EDTA, and 1 mM dithiothreitol at 37° C. The reaction is incubated for 1 h in a total volume of 0.4 ml then terminated with an equal volume of 5% tricbloroacetic acid. 0.1 ml of the internal standard 3,4dimiethylbenzoic acid (10 μg/ml) is added. Precipitated protein is separated by centrifugation at 12,000×g for 10 min. The supernatant is transferred to a clean tube and extracted twice with 0.4 ml of methylene chloride. The two extracts are combined and dried under a stream of nitrogen. The residue is resuspended in 14% acetonitrile, 250 mM KH₂PO₄, pH 4.0, with 8 μl of diethylamine per 100 ml and) and injected onto a C18 reverse-phase HPLC colunmn for separation. The colum eluate was monitored at 235 mn DME activity is quantified by comparing peak area ratios of the analyte to the internal standard. A standard curve was generated with benzoic acid standards prepared in a trichloroacetic acid-treated protein matrix (Evgenia, V. et al. (1997) J. Biol Chem. 272:14769-14775).

In another alternative, DME carboxyl esterase activity against the water-soluble substrate para-nitrophenyl butyric acid is determined by spectrophotometric methods well known to those skilled in the art. In this procedure, the DME-contalning saimples are diluted with 0.5 M Tris—HCl (pH 7.4 or 8.0) or sodium acetate (pH 5.0) in the presence of 6 mM taurocholate. The assay is initiated by adding a freshly prepared para-iitrophenyl butyric acid solution (100 μg/ml in sodium acetate, pH 5.0). Carboxyl esterase activity was then monitored and compared with control autohydrolysis of the substrate using an spectrophotometer set at 405 nm (Wan, L. et al (2000) J. Biol. Chein 275:10041-10046).

Heparan sulfate 6-sulfotransferase activity of DMB is measured in vitro by incubating a sample containing DME along with 2.5 μmol imidazole HCl (pH 6.8),3.75 μg of protamine chloride, 25 nmol (as hexosamine) of completely desulfated and N-resulfated heparn, and 50 pmol (about 5×10⁵ cpm) of [³⁵S] adenosine 3′-phosphate 5′-phosphosulfate (PAPS) in a final reaction volume of 50 μl at 37° C. for 20 min. The reaction is stopped by immersing the reaction tubes in a boiling water bath for 1 min. 0.1 μmol (as glucuronic acid) of chondroitin sulfate A is added to the reaction mixture as a carrier. ³⁵S-labeled polysaccharides are precipitated with 3 volumes of cold ethanol containing 1.3% potassium acetate and separated completely from unincorporated [³⁵S]PAPS and its degradation products by gel chromatography using desalting columns. One unit of enzyme activity is defined as the amount required to transfer 1 pmol of sulfate/rin. as determined by the amount of [³⁵S]PAPS incorporated into the precipitated polysaccharides (Habuchi, H. et al. (1995) 3. Biol. Chem. 270:4172-4179).

In the alternative, heparan sulfate 6-sulfotransferase activity of DME is measured by extraction and renaturation of enzyme from gels following separation by sodium dodecyl sulfate polyacrylamide gel electrophoresis (SDS-PAGE). Following separation, the gel is washed with 0.05 M Tris—HCl, pH 8.0, cut into 3-5 mm segments and subjected to agitation at 4° C. with 100 μl of 0.05 M Tris—HCl, pH 8.0 containing 0.15 M NaCl for 48 h. The eluted enzyme is collected by centrifigation and assayed for the sulfotransferase activity as above (Habuchi, H.et al. (1995) J. Biol. Chem. 270:4172-4179).

In another alternative, DME sulfotransferase activity is determined by measuring the transfer of [³⁵S]sulfate from [35S]PAPS to an immobilized peptide. In one example, the peptide (QATEYEYU)YDFLPEC) represents the N-terminal 15 residues of the mature P-selectin glycoprotein ligand-l polypeptide to which is added C-terminal cysteine residue. The peptide spans three potential tyrosine sulfation sites. The peptide is linked via the cysteine residue to iodoacetamide-activated resin at a density of 1.5-3.0 μmol peptide/ml of resin. The enzyme assay is performed by combining 10 μl of peptide-derivitized beads with 2-20 μl of DME-contaiing sample in 40 mM Pipes (pH 6.8), 0.3 M NaCl, 20 mM MnCl₂, 50 mM NaF, 1% Triton X-100, and 1 mM 5′-AMP in a final volume of 130 μl. The assay is initiated by. addition of 0.5 μCi of [³⁵S]pAPS (1.7 μM; 1 Ci=37 GBq). After 30 min at 37° C., the reaction beads are washed with 6 M guanidine at 65° C. and the radioactivity incorporated into the beads is determined by liquid scintillation counting. Transfer of [3⁵S]sulfate to the bead-associated peptide is measured to determine the DME activity in the sample. One unit of activity is defined as 1 pmol of product formed per min (Ouyang, Y -B. et al. (1998) Biochemistry 95:2896-2901).

In another alternative, DME sulfotransferase assays are performed using [³⁵S]PAPS as the sulfate donor in a final volume of 30 μl contains 50 mM Hepes-NaOH (pH 7.0), 250 mM sucrose, 1 mM dithiothreitol, 14 μM[³⁵S]PAPS (15 Ci/mmol), and dopamine (25 μM), p-nitrophenol (5 μM), or other candidate substrates. Assay reactions are started by the addition of a purified DME enzyme preparation or a sample containing DME activity, allowed to proceed for 15 min at 37° C., and terminated by heating at 100° C. for 3 mnin The precipitates formed are cleared by centrifugation. The supernatants are then subjected to the analysis of ³⁵S-sulfated product by either thin-layer chromatography or a two-dioensional thin layer separation procedure. Appropriate standards are run in parallel with the supematants to allow the identification of the ³⁵S-sulfated products and determine the enzyme specificity of the DME-containing samples based on relative relates of migration of reaction products (Sakakibara, Y. et al. (1998) J. Biol. Chem. 273:6242-6247).

Squalene epoxidase activity of DME is assayed in a mixture comprising purified DME (or a crude mixture comprising DME), 20 mM Tris—HCl (pH 7.5), 0.01 mM FAD, 0.2 unit of NADPH-cytochrome C (P-450) reductase, 0.01 mM [¹⁴C]squalene (dispersed with the aid of 20 μl of Tween 80), and 0.2% Triton X-100. 1 mM NADPH is added to initiate the reaction followed by incubation at 37° C. for 30 min. The nonsaponifiable lipids are analyzed by silica gel TLC developed with ethyl acetate/benzene (0.5:99.5, v/v). The reaction products are compared to those from a reaction mixture without DME. The presence of 2,3(S)-oxidosqualene is confirmed using appropriate lipid standards (Sakakibara, J. et al. (1995) 270:17-20).

Epoxide hydrolase activity of DME is determined by following substrate depletion using gas chromatographic (GC) analysis of ethereal extracts or by following substrate depletion and diol production by GC analysis of reaction mixtures quenched in acetone. A sample containing DME or an epoxide hydrolase control sample is incubated in 10 mM Tris—HCl (pH 8.0), 1 mM ethylenediaminetetraacetate (EDTA), and 5 mM epoxide substrate (e.g., ethylene oxide, styrene oxide, propylene oxide, isoprene monoxide, epichlorohydrin, epibromohydrin, epifliorohydrin, glycidol, 1,2-epoxybutane, 1,2-epoxyhexane, or 1,2-epoxyoctanea). A portion of the sample is withdrawn from the reaction mnxture at various time points, and added to 1 ml of ice-cold acetone containing an internal standard for GC analysis (e.g., 1-nonanol). Protein and salts are removed by centrifugation (15 min, 4000×g) and the extract is analyzed by GC using a 0.2 mm×25-m CP-Wax57-CB column (CHIROMPACK, Middelburg, The Netherlands) and a flame-ionization detector. The identification of GC products is performed using appropriate standards and controls well known to those skilled in the art. 1 Unit of DME activity is defined as the amount of enzyme that catalyzes the production of 1 μmol of diolimin (Rink, R et al. (1997) J. Biol. Chenl. 272:14650-14657).

Aminotransferase activity of DME is assayed by incubating samples containing DME for 1 hour at 37° C. in the presence of 1 mM L-kynurenine and 1 mM 2-oxoglutaratr in a final volume of 200 μl of 150 rnM Tris acetate buffer (pH 8.0) containing 70 μM PLP. The formation of kynurenic acid is quantified by HPLC with spectrophotometric detection at 330 nm using the appropriate standards and controls well known to those skilled in the art. In the alternative, L-3-hydroxykynurenine is used as substrate and the production of xanthurenic acid is determined by HPLC analysis of the products with Uv detection at 340 nm. The production of kynurenic acid xanthurenic acid, respectively, is indicative of aminotransferase acitity (Buchli, R. et al. (1995) J. Biol. Chem. 270:29330-29335).

In another alternative, aminotransferase activity of DME is measured by determining the activity of purified DME or crude samples containing DME toward various amino and oxo acid substrates under single turnover conditions by monitoring the changes in the UVIVIS absorption spectrum of the enzyme-bound cofactor, PLP. The reactions are performed at 25° C. in 50 mM 4-methylinorpholine (pH 7.5) containing 9 μM purified DME or DME containing samples and substrate to be tested (amino and oxo acid substrates). The half-reaction from amino acid to oxo acid is followed by measuring the decrease in absorbance at 360 nm and the increase in absorbance at 330 nmi due to the conversion of enzyme-bound PLP to PMP. The specificity and relative activity of DME is determined by the activity of the enzyme preparation against specific substrates (Vacca, R. A. et al. (1997) J. Biol. Chem. 272:21932-21937).

Superoxide dismutase activity of DME is assayed from cell pellets, culture supernatants, or purified protein preparations. Samples or lysates are resolved by electrophoresis on 15% non-denaturing polyacrylamide gels. The gels are incubated for 30 min in 2.5 mM nitro blue tetrazolium, followed by incubation for 20 min in 30 mM potassium phosphate, 30 mM TEMED, and 30 μM nboflavin (pH 7.9). Superoxide dismutase activity is visualized as white bands against a blue background, following illumination of the gels on a lightbox. Quantitation of superoxide dismutase activity is performed by densitometric scanning of the activity gels using the appropriate superoxide dismutase positive and negative controls (e.g., various amounts of commercially available E. coli superoxide dismutase (Harth, G. and Horwitz, M. A. (1999) J. Biol. Chem. 274:4281-4292).

Catechol-O-mnthyltransferase activity of DME is measured in a reaction mixture consisting of 50 mM Tris—HCl (pH 7.4), 1.2 mM MgCl₂, 200 μM SAM (S-adenosyl-L-retbionine) iodide (containing 0.5 μCi of [methyl-[³H]SAM), 1 mM dithiothreitol, and varying concentrations of catechol substrate (e.g., L-dopa, dopamnine, or DBA) in a final volume of 1.0 ml. The reaction is initiated by the addition of 250-500 μg of purified DME or crude DME-containing sample and performed at 37° C. for 30 min. The reaction is arrested by rapidly cooling on ice and immediately extracting with 7 ml of ice-cold n-heptane. Following centrifligation at 1000×g fbr 10 min, 3-ml aliquots of the organic extracts are analyzed for radioactivity content by liquid scintillation counting. The level of catechol-associated radioactivity in the organic phase is proportional to the activity catechol-O-methyltransferase activity of DME (Zhu, B. T. Liehr, J. G. (1996) 271:1357-1363).

DHFR activity of DME is determined spectrophotometrically at 15° C. by following the disappearance of NADPH at 340 nm (ε₃₄₀=11,800 M⁻¹ cm⁻¹). The standard assay mixture contains 100 μM NADPH, 14 mM 2-mercaptoethanol, MTEN buffer (50 mM 2-morpholinoethanesulfonic acid, 25 mM tris(hydroxymethyl)aminomethane, 25 mM ethanolamine, and 100 mM NaCl, pH 7.0), and DME in a final volume of 2.0 ml. The reaction is started by the addition of 50 μM dihydrofolate (as substrate). The oxidation of NADPH to NADP⁺ corresponds to the reduction of dihydrofolate in the reaction and is proportional to the amount of DHFR activity in the sample (Nakamura, Tₘ and Iwakura, M. (1999) J. Biol. Chem. 274:19041-19047).

Sulfotransferase activity of DME is measured using the incorporation of ³⁵S from [³⁵S]PAPS into a model substrate such as phenol (Folds, A. and Meek, J. L. (1973) Biochirr Biophys. Acta 327:365-374). An aliquot of enzyme is incubated at 37° C. with 1 mL of 10 mM phosphate buffer pH 6.4, 50 μM phenol, 0.4-4.0 μM [³⁵S]PAPS. After sufficient time for 5-20% of the radiolabel to be transferred to the substrate, 0.2 ml. of 0.1 M barium acetate is added to precipitate protein and phosphate buffer. Then 0.2 ml. of 0.1 M Ba(OH)₂ is added, followed by 0.2 ml. ZISO₄. The supernatant is cleared by centrifugation, which removes proteins as well as unreacted [³⁵S]PAPS. Radioactivity in the supernatant is measured by scintillation. The enzyme activity is determined from the number of moles of radioactivity in the reaction product.

XVIII. Identification of DME Inhibitors

Compounds to be tested are arrayed in the wells of a multi-well plate in varying concentrations along with an appropriate buffer and substrate, as described in the assays in Example XVII. DME activity is measured for each well and the ability of each compound to inhibit DME activity can be determined, as well as the dose-response profiles. This assay could also be used to identify molecules which enhance DME activity.

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the descnbed modes for caning out the invention which are obvious to those skilled in molecular biology or related fields are intended to be within the scope of the following claims.

TABLE 1

| Incyte | Poly- | Ineyte | Poly- | Incyte |
|---|---|---|---|---|
| Project | peptide | Polypeptide | nucleotide | Polynucleotide |
| ID | SEQ ID NO: | ID | SEQ ID NO: | ID |
| 7248285 | 1 | 7248285CD1 | 20 | 7248285CB1 |
| 7472835 | 2 | 7472835CD1 | 21 | 7472835CB1 |
| 7476203 | 3 | 7476203CD1 | 22 | 7476203CB1 |
| 7478583 | 4 | 7478583CD1 | 23 | 7478583CB1 |
| 7478585 | 5 | 7478585CD1 | 24 | 7478585CB1 |
| 7479904 | 6 | 7479904CD1 | 25 | 7479904CB1 |
| 7480367 | 7 | 7480367CD1 | 26 | 7480367CB1 |
| 8069390 | 8 | 8069390CD1 | 27 | 8069390CB1 |
| 7473869 | 9 | 7473869CD1 | 28 | 7473869CB1 |
| 7478588 | 10 | 7478588CD1 | 29 | 7478588CB1 |
| 55046125 | 11 | 55046125CD1 | 30 | 55046125CB1 |
| 3538709 | 12 | 3538709CD1 | 31 | 3538709CB1 |
| 71563101 | 13 | 71563101CD1 | 32 | 71563101CB1 |
| 7472027 | 14 | 7472027CD1 | 33 | 7472027CB1 |
| 7480358 | 15 | 7480358CD1 | 34 | 7480358CB1 |
| 1618256 | 16 | 1618256CD1 | 35 | 1618256CB1 |
| 3387823 | 17 | 3387823CD1 | 36 | 3387823CB1 |
| 55142051 | 18 | 55142051CD1 | 37 | 55142051CB1 |
| 7395274 | 19 | 7395274CD1 | 38 | 7395274CB1 |

TABLE 2

| Polypeptide | Incyte | GenBank | Probability | |
|---|---|---|---|---|
| SEQ ID NO: | Polypeptide ID | ID NO: | score | GenBank Homolog |
| 1 | 7248285CD1 | g4929199 | 1.90E−248 | [Perca flavescens] lysyl oxidase |
| | | | | related protein homolog |
| | | | | Langenau, D. M. et al. (1999) J. Mol. |
| | | | | Endocrinol. 23:137-152. |
| 2 | 7472835CD1 | g559046 | 4.50E−181 | [Homo sapiens] flavin-containing |
| | | | | monooxygenase 5 |
| | | | | Overby, L. H. et al. (1995) Arch. |
| | | | | Biochem. Biophys. 317:275-284. |
| 3 | 7476203CD1 | g2653663 | 5.30E−91 | [Mus musculus] cytochrome P450 |
| | | | | monooxygenase |
| 4 | 7478583CD1 | g14475602 | 3.00E−72 | [fl] [Bacillus cereus] acetylacetoin |
| | | | | reductase |
| 4 | 7478583CD1 | g529564 | 2.00E−61 | “[Pseudomonas putida] 2,3−butanediol |
| | | | | dehydrogenase” |
| | | | | Huang, M. et al. (1994) FEMS |
| | | | | Microbiol. Lett. 124:141-150. |
| 5 | 7478585CD1 | g14582559 | 1.00E−125 | [fl] [Staphylococcus aureus] |
| | | | | (AF282920) guinone oxidoreductase |
| 6 | 7479904CD1 | g6683558 | 2.30E−255 | [Mus musculus] heparan sulfate 6- |
| | | | | sulfotransferase 2 |
| 7 | 7480367CD1 | g242749 | 1.30E−101 | [Homo sapiens] glutathione S- |
| | | | | transferase Ha1 subunit [EC |
| | | | | 2.5.1.18] |
| | | | | Rozen, F. et al. (1992) Arch. |
| | | | | Biochem. Biophys. 292:589-593. |
| 8 | 8069390CD1 | g5531815 | 1.90E−56 | [Homo sapiens] steroid dehydrogenase |
| | | | | homolog |
| 8 | 8069390CD1 | g13183088 | 1.00E−179 | [fl] [Homo sapiens] steroid |
| | | | | dehydrogenase-like protein |
| 9 | 7473869CD1 | g1433l125 | 2.00E−78 | [fl] [Canis familiaris] |
| | | | | carboxylesterase D1 |
| 10 | 7478588CD1 | g14331125 | 1.00E−140 | [fl] [Canis familiaris] |
| | | | | carboxylesterase D1 |
| 11 | 55046125CD1 | g6683558 | 1.80E−236 | [Mus musculus] heparan sulfate 6- |
| | | | | sulfotransferase 2 |
| 12 | 3538709CD1 | g2633182 | 1.30E−11 | [Bacillus subtilis] similar to |
| | | | | epoxide hydrolase |
| | | | | Kunst, F. et al. Nature 390:249-256. |
| 12 | 35387090D1 | g2649734 | 2.00E−14 | [Archaeoglobus fulgidus] |
| | | | | carboxylesterase (est-1) |
| 13 | 71563101CD1 | g1944136 | 1.10E−234 | [Rattus norvegicus] beta-alanine- |
| | | | | pyruvate aminotransferase |
| 14 | 7472027CD1 | g1151081 | 5.40E−34 | [Cavia porcellus] hydroxysteroid |
| | | | | sulfotransferase 2 |
| | | | | Luu, N.X. (1995) Biochem. Biophys. |
| | | | | Res. Commun. 217:1078-1086. |
| 15 | 7480358CD1 | g1066120 | 2.10E−25 | [Cavia porcellus] copper-zinc |
| | | | | superoxide dismutase |
| | | | | Yuan, H.T. et al. (1996) Biochim. |
| | | | | Biophys. Acta 1305:163-171. |
| 16 | 1618256CD1 | g10880453 | 2.00E−68 | [fl] [Homo sapiens] glycoprotein |
| | | | | beta-Gal 3′-sulfotransferase |
| 17 | 3387823CD1 | g6782275 | 3.00E−99 | [Caenorhabditis elegans] similar to |
| | | | | monoamine oxidase˜cDNA EST |
| | | | | The C. elegans Sequencing |
| | | | | Consortium. Genome sequence of the |
| | | | | nematode C. elegans: a platform for |
| | | | | investigating biology. Science |
| | | | | 282:2012-2018. |
| 18 | 55142051CD1 | g179955 | 9.50E−41 | [Homo sapiens] catechol-O- |
| | | | | methyltransferase |
| | | | | Bertocci, B. et al. (1991) Human |
| | | | | catechol-O-methyltransferase: |
| | | | | cloning and expression of the |
| | | | | membrane-associated form. Proc. |
| | | | | Natl. Acad. Sci. U.S.A. 88:1416- |
| | | | | 1420. |
| 19 | 7395274CD1 | g5852342 | 4.60E−99 | [Fundulus heteroclitus] cytochrome |
| | | | | P450 2N1 |
| | | | | Oleksiak, M. F. et al. (2000) |
| | | | | Identification, functional |
| | | | | characterization, and regulation of |
| | | | | a new cytochrome P450 subfamily, the |
| | | | | CYP2Ns. J. Biol. Chem. 275:2312- |
| | | | | 2321. |

TABLE 3

| SEQ | Incyte | Amino | Potential | Potential | | Analytical |
|---|---|---|---|---|---|---|
| ID | Polypeptide | Acid | Phosphorylation | Glycosylation | Signature Sequences, | Methods and |
| NO: | ID | Residues | Sites | Sites | Domains and Motifs | Databases |
| 1 | 7248285CD1 | 756 | T29, S39, T56, | N198, N629 | OXIDASE; LYSINE; LYSYL; COPPER DOMAIN; | BLAST-DOMO |
| | | | S85, S107, | | DM04978\|Q05063\|1-419: Q505-C731 | |
| | | | S123, S167, | | LYSYL OXIDASE PROTEINLYSINE PRECURSOR | BLAST-PRODOM |
| | | | T239, S304, | | SIGNAL 6OXIDASE OXIDOREDUCTASE COPPER | |
| | | | T334, S356, | | GLYCOPROTEIN HOMOLOG: PD012364: P533-C731 | |
| | | | T386, T484, | | Lysyl oxidase signature: PR00074: R593- | BLIMPS-PRINTS |
| | | | S498, S527, | | T621, T621-C648, D679-E707, S708-N735 | |
| | | | S531, T600, | | Signal peptide (Signal_peptide) : M1-P24 | HMMER |
| | | | T621, T673 | | Lysyl oxidase Lysyl_oxidase: P533-S736 | HMMER-PFAM |
| | | | | | Signal cleavage: M1-P24 | SPSCAN |
| 2 | 7472835CD1 | 544 | T30, S49, T63, | N47, N62, | DIMETYLANILINE MONOOXYGENASE (N-OXIDE- | BLAST-DOMO |
| | | | S113, S179, | N489 | FORMING) DM02407\|P49109\|1-335: K3-F337 | |
| | | | S184, S196, | | FLAVIN-CONTAINING MONOOX PR00370: A5-K20, | BLIMPS-PRINTS |
| | | | S215, T216, | | E28-K46, F141-P157, K186-L204, N323-D341, | |
| | | | S220, Y231, | | G377-G396, Y474-R495 | |
| | | | T285, S296, | | Pyridine nucleotide disulphide reductase | BLIMPS-PRINTS |
| | | | S304, T309, | | class-Il signature: PR00469: R187-K209, | |
| | | | S318, T319, | | D325-Y333, F183-V207, V326-T347 | |
| | | | S338, T347, | | Flavin-binding monooxygenase-like FMO-like: | HMMER-PFAM |
| | | | Y421, T424, | | K3-F535 | |
| | | | T491, T505 | | Pyridine nucleotide-disulphide | PROFILESCAN |
| | | | | | oxidoreductases class-Il active site | |
| | | | | | (pyridine_redox_2.prf): P157-L214 | |
| | | | | | Signal Cleavage:M1-A18 | SPSCAN |
| 3 | 7476203CD1 | 501 | S54, T118, | | CYTOCHROME P450 DM00022\|P52786\|83-492: S71- | BLAST-DOMO |
| | | | T174, S372, | | P478 | |
| | | | T402, S412, | | CYTOCHROME P450 MONOOXYGENASE | BLAST-PRODOM |
| | | | T454 | | OXIDOREDUCTASE HEME ELECTRON TRANS- | |
| | | | | | PORT MEMBRANE MICROSOME ENDOPLASMIC: | |
| | | | | | PD000021: Q237-L351, D396-P464, P31-K60, | |
| | | | | | E339-F411, N108-E158 | |
| | | | | | P450 superfamily signature: PR00385: A349- | BLIMPS-PRINTS |
| | | | | | V360, L425-Y434, G296-I313, Q314-D327 | |
| | | | | | Mitochondrial P450 signature: PR00408: | BLIMPS-PRINTS |
| | | | | | S114-R124, G296-I313, K342-V360 | |
| | | | | | E-class P450 group I: PR00463: S173-D191, | BLIMPS-PRINTS |
| | | | | | N285-A302, L305-G331, M348-V366, N389-D413, | |
| | | | | | F424-Y434, Y434-F457 | |
| | | | | | Signal peptide (signal_peptide): M1-C24 | HMMER |
| | | | | | Transmembrane domain (transmem_domain): L4- | HMMER |
| | | | | | F23, L197-Q214 | |
| | | | | | Cytochrome P450 (p450): P31-M67, R107-A485 | HMMER-PFAM |
| | | | | | Cytochrome P450 cysteine heme-iron ligand | PROFILESCAN |
| | | | | | signature (cytochrome_p450.prf): F406-R456 | |
| | | | | | Signal cleavage: M1-Q49 | SPSCAN |
| 4 | 7478583CD1 | 345 | S143 S219 S251 | N141 | ZINC-CONTAINING ALCOHOL DEHYDROGEN- | BLAST-DOMO |
| | | | S327 S99 T248 | | ASES DM00064\|P39714\|18-373: E105-I343, E21-599 | |
| | | | T53 | | OXIDOREDUCTASE ZINC DEHYDROGENASE AL- | BLAST-PRODOM |
| | | | | | COHOL NAD PROTEIN FAMILY MULTIGENE | |
| | | | | | NADP FORMALDEHYDE: PD000104: A3-V265, | |
| | | | | | G168-A246 | |
| | | | | | Zinc-containing alcohol dehydrogenase | BLIMPS-BLOCKS |
| | | | | | proteins: BL00059: E26-H42, P65-V92, G117- | |
| | | | | | Q158, G168-T214 | |
| | | | | | Zinc-binding dehydrogenases (adh_zinc): K2- | HMMER-PFAM |
| | | | | | K344 | |
| | | | | | Adh_Zinc G69-V83 | MOTIFS |
| | | | | | Zinc-containing alcohol dehydrogenase | PROFILESCAN |
| | | | | | signatures (adh_zinc.prf): G38-E102 | |
| 5 | 7478585CD1 | 361 | T14, Y86, S107, | N81 | ZINC-CONTAINING ALCOHOL DEHYDROGEN- | BLAST-DOMO |
| | | | S113, T330 | | ASES DM00064\|S57611\|3-340: Q32-V356 | |
| | | | | | Zinc-binding dehydrogenases (adh_zinc): | HMMER-PFAM |
| | | | | | D46-K357 (E-value = 4.4e−06, Score = −68.6) | |
| | | | | | Quinone oxidoreductase: BL01162: S150-K193 | BLIMPS-BLOCKS |
| | | | | | (p < 0.057) | |
| 6 | 7479904CD1 | 499 | S5, S50, S55, | N63, N204, | HEPARANSULFATE 6-SULFOTRANSFERASE | BLAST-PRODOM |
| | | | T65, T120, | N298, N354, | TRANSFERASE: PD139786: K207-R412, M1-K182 | |
| | | | T129, S156, | N438, N450, | Signal peptide (signal_peptide) : M1-Q24 | HMMER |
| | | | S187, S199, | N458, N483, | Transmembrane domain (transmem_domain): L8- | HNMER |
| | | | T205, S208, | N486 | Y25 | |
| | | | T237, T251, | | Signal cleavage: M1-Q24 | SPSCAN |
| | | | T260, Y277, | | | |
| | | | S314, T355, | | | |
| | | | S359, S464, | | | |
| | | | S471, S476 | | | |
| 7 | 7480367CD1 | 222 | | | GLUTATHIONE TRANSFERASE DM00127\|P08263\| | BLAST-DOMO |
| | | | | | 71-190: L72-P192 | |
| | | | | | GLUTATHIONE TRANSFERASE STRANSFERASE | BLAST-PRODOM |
| | | | | | MULTIGENE FAMILY PROTEIN CLASS-ALPHA S- | |
| | | | | | CRYSTALLIN GST LYASE: PD000312: L81-R204, | |
| | | | | | K6-L102 (p = 8.0e−10) | |
| | | | | | Glutathione S-transferase: PF00043: Q54-G83 | BLIMPS-PFAM |
| | | | | | Glutathione S-transferases: L7-P192 | HMMER-PFAM |
| 8 | 8069390CD1 | 330 | T33, S58, S98, | N318 | SHORT-CHAIN ALCOHOL DEHYDROGENASE | BLAST-DOMO |
| | | | T114, S128, | | FAMILY DM00034\|P37058\|43-275: | |
| | | | S271, S286, | | I63-I297 | |
| | | | T290, S320 | | Short-chain dehydrogenas: BL00061: G198- | BLIMPS-BLOCKS |
| | | | | | E235 | |
| | | | | | Alcohol dehydrogenase: PR00080: F218-A237, | BLIMPS-PRINTS |
| | | | | | K143-V154, G198-G206 | |
| | | | | | Glucose/ribitol dehydrogenase: PR00081: | BLIMPS-PRINTS |
| | | | | | W69-E86, K143-V154, M192-C208, F218-A237, | |
| | | | | | K239-T256 | |
| | | | | | Short chain dehydrogenase (adh_short): R68- | HMMER-PFAM |
| | | | | | S254 | |
| | | | | | Short-chain alcohol dehydrogenase family | PROFILESCAN |
| | | | | | signature (adh_short.prf): G198-P248 | |
| | | | | | Signal cleavage: M1-K36 | SPSCAN |
| 9 | 7473869CD1 | 303 | S4, Y20, S52, | N116, N178 | CARBOXYLESTERASES TYPE-B: | BLAST-DOMO |
| | | | S81, S199, | | DM00175\|P16303\|20-361: M1-M264 | |
| | | | T277, S284 | | ESTERASE HYDROLASE PRECURSOR SIGNAL | BLAST-PRODOM |
| | | | | | GLYCOPROTEIN SERINE PROTEIN | |
| | | | | | CARBOXYLESTERASE FAMILY MULTIGENE | |
| | | | | | PD000169: S4-M281 | |
| | | | | | Carboxylesterases type-B: BL00122: E16-P26, | BLIMPS-BLOCKS |
| | | | | | P36-F46, V65-L80, G89-I129, L141-G150 | |
| | | | | | Carboxylesterases (COesterase): L12-F266 | HMMER-PFAM |
| | | | | | Carboxylesterase motifs: | MOTIFS |
| | | | | | (carboxylesterase_B_1): F110-G125 | |
| | | | | | (carboxylesterase_B_2): E16-P26 | |
| | | | | | Carboxylesterases type-B signatures | PROFILESCAN |
| | | | | | (carboxylesterase_b.prf): N90-A145 | |
| 10 | 7478588CD1 | 584 | S23, T47, Y107, | N237, N299, | CARBOXYLESTERASES TYPE-B DM00175\|Q04791\| | BLAST-DOMO |
| | | | S125, Y141, | N411 | 26-353: K48-M385 | |
| | | | S173, S202, | | ESTERASE HYDROLASE PRECURSOR SIGNAL | BLAST-PRODOM |
| | | | S320, T398, | | GLYCOPROTEIN SERINE PROTEIN | |
| | | | S405, Y548, | | CARBOXYLESTERASE FAMILY MULTIGENE: | |
| | | | T554, S575 | | PD000169: M37-E430, W26-T203, M394-W569, | |
| | | | | | A488-P578 | |
| | | | | | Carboxylesterases type-B: BL00122: F74-P94, | BLIMPS-BLOCKS |
| | | | | | E137-P147, P157-F167, V186-L201, G210-I250, | |
| | | | | | L262-G271, W522-N532 | |
| | | | | | Carboxylesterases COesterase: R25-W569 | HMMER-PFAN |
| | | | | | Carboxylesterase_B_1: F231-G246; | MOTIFS |
| | | | | | Carboxylesterase_B_2: E137-P147 | |
| | | | | | Carboxylesterases type-B signatures | PROFILESCAN |
| | | | | | (carboxylesterase_b.prf): N211-A266 | |
| | | | | | Signal cleavage: M1-G43 | SPSCAN |
| 11 | 55046125CD1 | 508 | S28, S54, S99, | N112, N307, | HEPARANSULFATE 6-SULFOTRANSFERASE | BLAST-PRODOM |
| | | | S104, T114, | N363, M447, | TRANSFERASE: PD139786:M50-R421 | |
| | | | T169, T178, | N459, N467, | Transmembrane domain (transmem_domain): | HMMER |
| | | | S205, T246, | N492, N495 | L57-Y74 | |
| | | | T260, T269, | | | |
| | | | Y286, S323, | | | |
| | | | T364, S368, | | | |
| | | | S473, S480, | | | |
| | | | S485, | | | |
| 12 | 3538709CD1 | 439 | T34, T83, T167, | N108, N332 | Alpha/beta hydrolase fold: PR00111: G202- | BLIMPS-PRINTS |
| | | | S168, T281, | | S217., A262-I275, V362-E376 | |
| | | | S423 | | Epoxide hydrolase signature: PR00412: H182- | BLIMPS-PRINTS |
| | | | | | R200, G202-S217, L248-L261, I393-F415 | |
| | | | | | Alpha/beta hydrolase fold (abhydrolase): | HMMER-PFAM |
| | | | | | Y203-L416 | |
| 13 | 71563101CD1 | 514 | T32, S169, | | AMINOTRANSFERASES CLASS-III PYRIDOXAL- | BLAST-DOMO |
| | | | S192, T248, | | PHOSPHATE ATTACHMENT DM00188\|P16932\| | |
| | | | S253, T363, | | 2-426: D271-A504, K84-R233 | |
| | | | S448, T480, | | ALANINEGLYOXYLATE AMINOTRANSFERASE 2 | BLAST-PRODOM |
| | | | T490 | | PRECURSOR EC 2.6.1.44 AGT | |
| | | | | | BETAALANINEPYRUVATE BETAALAAT II | |
| | | | | | TRANSFERASE PYRIDOXAL PHOSPHATE | |
| | | | | | MITOCHONDRION TRANSIT PEPTIDE: PD032383: | |
| | | | | | M1-E94 | |
| | | | | | Aminotransferases class-3: BL00600: P343- | BLIMPS-BLOCKS |
| | | | | | G355, H377-L395, D98-P121, P160-S185, I190- | |
| | | | | | L205, I281-G294, F302-G330 | |
| | | | | | Transmembrane domain: Y104-N124, | HMMER |
| | | | | | Aminotransferases class-III pyridoxal- | HMMER-PFAM |
| | | | | | phosphate: aminotran_3: W95-T208 I281-M487 | |
| | | | | | Aminotransferases class-III pyridoxal- | PROFILESCAN |
| | | | | | phosphate attachment site | |
| | | | | | (aa_transfer_class_3.prf): E308-C374 | |
| 14 | 7472027CD1 | 226 | S17, T23, T28, | N21, N66 | PHOSPHOADENOSINE-PHOSPHOSULFATE SYN- | BLAST-DOMO |
| | | | S106, S136, | | THETASE (PAPS) BINDING SITE BINDING | |
| | | | T195 | | DM00981\|JC4531\|1-287: M50-E193 | |
| | | | | | TRANSFERASE SULFOTRANSFERASE STEROID | BLAST-PRODOM |
| | | | | | METABOLISM HYDROXYSTEROID ALCOHOL | |
| | | | | | PHENOL ESTROGEN PROTEIN STEROIDBINDING | |
| | | | | | PD001218: L73-Yl65 | |
| 15 | 7480358CD1 | 121 | T38, S96, T119 | N69 | COPPER/ZINC SUPEROXIDE DISMUTASE | BLAST-DOMO |
| | | | | | DM00227\|P00442\|3-147: K3-V116 | |
| | | | | | SUPEROXIDE DISMUTASE CUZN OXIDOREDUC- | BLAST-PRODOM |
| | | | | | TASE COPPER ZINC PRECURSOR SIGNAL PERI- | |
| | | | | | PLASMIC EXTRACELLULAR: PD000469: M2-V116 | |
| | | | | | Copper/Zinc superoxide dismutase proteins: | BLIMPS-BLOCKS |
| | | | | | BL00087: K3-Q22, R40-W83 | |
| | | | | | Cu—Zn-superoxide dismutase: PR00068: R43- | BLIMPS-PRINTS |
| | | | | | P65, V97-T119 | |
| | | | | | Copper/zinc superoxide dismutase (SODC): | HMMER-PFAM |
| | | | | | (sodcu): M2-P73, S96-V116 | |
| 16 | 1618256CD1 | 486 | T9, S55, T74, | N374 | Signal peptide: M1-G29 | SIGPEPT |
| | | | T265, S296, | | Signal cleavage: M1-G36 | SPSCAN |
| | | | T349, T356, | | CEREBROSIDE SULFOTRANSFERASE: PD140372: | BLAST-PRODOM |
| | | | T420, S431, | | W13-G215, F279-Y461 | |
| | | | S444, T480 | | | |
| 17 | 3387823CD1 | 649 | T64, T184, | N209 | PROTEIN SIMILARITY KINASE SLIME MOLD | BLAST-PRODOM |
| | | | T236, S253, | | R13G10.2 CHROMOSOME III OXIDOREDUCTASE | |
| | | | S368, S382, | | FLAVOPROTEIN: PD041935: L358-D433, D250- | |
| | | | S431, T438. | | G337 | |
| | | | S471, S534, | | Signal peptide: M1-E22 | SIGPEPT |
| | | | S356, S516, | | Flavin containing amine oxidase: | HMMER-PFAM |
| | | | S547, T550, | | Amino oxidase: P219-A647 | |
| | | | T563, T624 | | Adrenodoxin reductase family (flavin- | BLIMPS-PRINTS |
| | | | | | containing component of mitochondrial | |
| | | | | | monooxygenase system): PR00419A: S211-I233, | |
| | | | | | PR00419B: K234-R247, PR00419D: V212-R226 | |
| | | | | | Aromatic-ring hydroxylas (flavoprotein | BLIMPS-PRINTS |
| | | | | | monooxygenase): PR00420A: S211-I233, | |
| | | | | | PR00420C: Q614-P629 | |
| 18 | 55142051CD1 | 258 | S35, T67, S73, | | Transmembrane domain: M1-V18 | HMMER |
| | | | S74, S159, S174 | | O-METHYLTRANSFERASE TRANSFERASE | BLAST-PRODOM |
| | | | | | METHYLTRANSFERASE: PD173699: I65-D162 | |
| | | | | | (P = 9.5e−10) | |
| | | | | | CATECHOL-O-METHYLTRANSFERASE CHAIN | BLAST-DOMO |
| | | | | | DM02481\|P22734\|41-139: E46-A137 | |
| 19 | 7395274CD1 | 544 | S85, S88, S135, | N331, N334, | CYTOCHROME P450 DM00022\|P10635\|52-480: | BLAST-DOMO |
| | | | S148, S181, | N538 | L110-F528 | |
| | | | T184, Y205, | | CYTOCHROME P450 MONOOXYGENASE | BLAST-PRODOM |
| | | | T244, S335, | | OXIDOREDUCTASE HEME ELECTRON TRANS- | |
| | | | S336, S393, | | PORT MEMBRANE MICROSOME ENDOPLASMIC | |
| | | | T395, T406, | | PD000021: K285-G433, I386-Q471, H450-I539, | |
| | | | T479, S519, | | I436-I539, A227-D338, G58-I231 | |
| | | | T525, S542 | | Cytochrome P450 cysteine BL00086: F480- | BLIMPS-BLOCKS |
| | | | | | F511 | |
| | | | | | Mitochondrial P450 signa PR00408:L370- | BLIMPS_PRINTS |
| | | | | | E383, A398-V416, F462-D470, I481-C490, | |
| | | | | | C490-F501, W50-P65, H169-K179, A352-S369 | |
| | | | | | E−class P450 group I PR00463: A111-L130, | BLIMPS-PRINTS |
| | | | | | S135-I156, A227-N245, Y341-T358, L361-G387, | |
| | | | | | E404-P422, N445-D469, P480-C490, C490-F513 | |
| | | | | | E-class P450 group IV PR00465: V105-V128, | BLIMPS-PRINTS |
| | | | | | H450-L468, L474-C490, C490-M508 | |
| | | | | | Cytochrome P450: L107-R543 | HMMER-PFAM |
| | | | | | Cytochrome P450: F483-G492 | MOTIFS |
| | | | | | Cytochrome P450 cysteine heme-iron ligand | PROFILESCAN |
| | | | | | signature (cytochrome_p450.prf): P464-A512 | |
| | | | | | Signal_cleavage: M1-G47 | SPSCAN |

TABLE 4

| Polynucleotide | Incyte | Sequence | Selected | | | |
|---|---|---|---|---|---|---|
| SEQ ID NO: | Polynucleotide ID | Length | Fragment(s) | Sequence Fragments | 5′ Position | 3′ Position |
| 20 | 7248285CB1 | 2603 | 2496-2603, | 7690384J1 (PROSTME06) | 652 | 1330 |
| | | | 526-572 | 6460535F7 (OSTEUNC01) | 1144 | 1999 |
| | | | | 8035282H1 (SMCRUNE01) | 276 | 892 |
| | | | | 2796172F6 (NPOLNOT01) | 2072 | 2603 |
| | | | | 7690384H1 (PROSTME06) | 1650 | 2185 |
| | | | | FL7248285_g7939436_000006 | 1106 | 1428 |
| | | | | _g1890108_1_7-8 | | |
| | | | | GNN:g7939436_000006_002.e | 1 | 456 |
| | | | | dit | | |
| 21 | 7472835CB1 | 1745 | 1-57, 412- | GBI.g7107731_0001.edit_2. | 1445 | 1745 |
| | | | 1745 | comp | | |
| | | | | GBI.g7107731_0001.edit_1 | 1 | 1674 |
| 22 | 7476203CB1 | 1587 | 1-209, 280- | GBI.g8567322_000003_00000 | 1 | 1139 |
| | | | 1587 | 4_edit | | |
| | | | | GBI.g8567322_000003_00000 | 960 | 1587 |
| | | | | 4_edit_2 | | |
| 23 | 7478583CB1 | 1038 | 1-1038 | GNN.g8576127_000058_002 | 1 | 1038 |
| 24 | 7478585CB1 | 1584 | 1-1584 | GNN.g8576127_CDS_1.edit | 1 | 1467 |
| | | | | GNN.7478585CT1 | 596 | 1584 |
| 25 | 7479904CB1 | 1500 | 1-149 | g1655588_g3191972_g856919 | 1 | 1500 |
| | | | | 3_edit | | |
| 26 | 7480367CB1 | 669 | | GBI:g8348076_edit_1 | 1 | 669 |
| | | | | 2090065H1 (PANCNOT04) | 508 | 669 |
| 27 | 8069390CB1 | 3551 | 1609-1783, | 7360250H1 (BRAIFEE05) | 607 | 1056 |
| | | | 701-898, 1- | 7660957H1 (OVARNOE02) | 2157 | 2645 |
| | | | 21, 3101-3551 | 7660957J1 (OVARNOE02) | 1 | 537 |
| | | | | 6450195H1 (BRAINOC01) | 330 | 989 |
| | | | | 1759470R6 (PITUNOT03) | 2966 | 3512 |
| | | | | 1619329T6 (BRAITUT12) | 2859 | 3484 |
| | | | | 4249653R6 (BRADDIR01) | 2357 | 2781 |
| | | | | g1203094 | 3095 | 3551 |
| | | | | 1576067T6 (LNODNOT03) | 2736 | 3390 |
| | | | | 7365248H1 (OVARDIC01) | 920 | 1560 |
| | | | | 4249653F6 (BRADDIR01) | 1748 | 2196 |
| | | | | 7100733H1 (BRAWTDR02) | 1597 | 2148 |
| | | | | 3333887F6 (BRAIFET01) | 1068 | 1673 |
| 28 | 7473869CB1 | 2178 | 1-231, 1549- | 55023671J1 | 1360 | 2178 |
| | | | 1579, 1949- | 55051441J1 | 1 | 1032 |
| | | | 2178, 654-732 | 55071187J1 | 1045 | 1662 |
| | | | | 55071183H1 | 987 | 1606 |
| 29 | 7478588CB1 | 2081 | 1-97, 1256- | 55017748J1 | 1496 | 1970 |
| | | | 1450 | 7580667H1 (BRAIFEC01) | 543 | 1108 |
| | | | | 60251275D1 | 1333 | 1837 |
| | | | | 55058627J1 | 157 | 714 |
| | | | | 6205079H1 (PITUNON01) | 1562 | 2081 |
| | | | | 4365390H1 (SKIRNOT01) | 1129 | 1391 |
| | | | | 55111596H1 | 1 | 540 |
| | | | | 55071187J1 | 752 | 1365 |
| 30 | 55046125CB1 | 2642 | 2250-2642, | 71975318V1 | 1694 | 2315 |
| | | | 403-468 | 2783360F6 (BRSTNOT13) | 2121 | 2642 |
| | | | | 55071237J1 | 1 | 548 |
| | | | | 55046165J2 | 720 | 1572 |
| | | | | 55046125H1 | 314 | 1143 |
| | | | | 5626585H1 (PLACFER01) | 1512 | 2108 |
| 31 | 3538709CB1 | 2080 | 863-1020, | 7067524H1 (BRATNOR01) | 624 | 1178 |
| | | | 2027-2080, | 2225614F6 (SEMVNOT01) | 1797 | 2080 |
| | | | 580-639 | 1668310H1 (BMARNOT03) | 1141 | 1376 |
| | | | | 3763474H1 (BRAINOT21) | 1166 | 1461 |
| | | | | 7764424H1 (URETTUE01) | 1 | 463 |
| | | | | 6883439J1 (BRAHTDR03) | 1315 | 1932 |
| | | | | 7191721H2 (DRATDIC01) | 253 | 855 |
| 32 | 71563101CB1 | 2219 | 1-44, 541- | 2560864H1 (ADRETUT01) | 1656 | 1947 |
| | | | 1063 | g2985189 | 1734 | 2212 |
| | | | | 71563534V1 | 684 | 1366 |
| | | | | 71562178V1 | 1184 | 1840 |
| | | | | 6926859H1 (SINITMR01) | 1 | 662 |
| | | | | 293718H1 (LIVRNOT04) | 1828 | 2066 |
| | | | | 71563101V1 | 529 | 1354 |
| 33 | 7472027CB1 | 681 | 148-681 | FL7472027CT1_00001 | 1 | 681 |
| 34 | 7480358CB1 | 399 | 1-35 | GNN.g9211248_000026_002 | 1 | 399 |
| 35 | 1618256CB1 | 2302 | 2241-2302, 1- | 7010610H1 (COLNFEC01) | 1 | 604 |
| | | | 260, 1172- | 1618256F6 (BRAITUT12) | 1329 | 1876 |
| | | | 1194 | 6330412F8 (BRANDIN01) | 359 | 1086 |
| | | | | 7403247H1 (SINIDME01) | 1475 | 2112 |
| | | | | 4922615F9 (TESTNOT11) | 646 | 1374 |
| | | | | 2605131F6 (LUNGTUT07) | 1835 | 2302 |
| 36 | 3387823CB1 | 3341 | 1-447, 3270- | 7741282H1 (THYMNOE01) | 1 | 662 |
| | | | 3341, 878- | 72335796V1 | 2819 | 3341 |
| | | | 1415 | 70858392V1 | 1919 | 2549 |
| | | | | 70857153V1 | 2459 | 2872 |
| | | | | 70856101V1 | 1381 | 1902 |
| | | | | 657765H1 (EOSINOT03) | 2322 | 2590 |
| | | | | 70002313D1 | 1551 | 2004 |
| | | | | 995958H1 (KIDNTUT01) | 2584 | 2902 |
| | | | | 6771554J1 (BRAUNOR01) | 768 | 1218 |
| | | | | 70857616V1 | 1116 | 1524 |
| | | | | 7087862H1 (BRAUTDRO3) | 438 | 906 |
| 37 | 55142051CB1 | 777 | 1-257 | 55142051J1 | 72 | 337 |
| | | | | GBI.g8567879_edit | 1 | 777 |
| 38 | 7395274CB1 | 3600 | 1-1390, 2414- | 6779264H1 (OVARDIR01) | 2278 | 2967 |
| | | | 3076, 2342- | g2064300 | 3001 | 3596 |
| | | | 2372, 3576- | 55052831H1 | 787 | 1597 |
| | | | 3600 | 7394114R8 (BRABDIE02) | 454 | 1371 |
| | | | | 8128245H1 (SCOMDIC01) | 1 | 693 |
| | | | | 2300407R6 (BRSTNOT05) | 2076 | 2545 |
| | | | | 2300407T6 (BRSTNOT05) | 3044 | 3568 |
| | | | | 7996595H1 (ADRETUC01) | 1493 | 2221 |
| | | | | g2079829 | 3243 | 3600 |
| | | | | 7458912H1 (LIVRTUE01) | 1677 | 2255 |
| | | | | 1259839T6 (MENITUT03) | 2869 | 3567 |
| | | | | 6544865H1 (LNODNON02) | 3124 | 3590 |

TABLE 5

| Polynucleotide | Incyte | Representative |
|---|---|---|
| SEQ ID NO: | Project ID | Library |
| 20 | 7248285CB1 | PROSTME06 |
| 26 | 7480367CB1 | SINTNOT02 |
| 27 | 8069390CB1 | HNT2AGT01 |
| 28 | 7473869CB1 | BRAQTDR02 |
| 29 | 7478588CB1 | OVARDIR01 |
| 30 | 55046125CB1 | BRSTNOT13 |
| 31 | 3538709CB1 | BRAHTDR03 |
| 32 | 71563101CB1 | SINITMR01 |
| 35 | 1618256CB1 | BRAITUT12 |
| 36 | 3387823CB1 | BRAINOT19 |
| 38 | 7395274CB1 | BRABDIE02 |

TABLE 6

| Library | Vector | Library Description |
|---|---|---|
| BRABDIE02 | pINCY | This 5′ biased random primed library was constructed using RNA isolated from diseased |
| | | cerebellum tissue removed from the brain of a 57-year-old Caucasian male who died from a |
| | | cerebrovascular accident. Serologies were negative. Patient history included |
| | | Huntington's disease, emphysema, and tobacco abuse (3-4 packs per day, for 40 years). |
| BRAHTDR03 | PCDNA2.1 | This random primed library was constructed using RNA isolated from archaecortex, |
| | | anterior hippocampus tissue removed from a 55-year-old Caucasian female who died from |
| | | cholangiocarcinoma. Pathology indicated mild meningeal fibrosis predominately over the |
| | | convexities, scattered axonal spheroids in the white matter of the cingulate cortex and |
| | | the thalamus, and a few scattered neurofibrillary tangles in the entorhinal cortex and |
| | | the periaqueductal gray region. Pathology for the associated tumor tissue indicated |
| | | well-differentiated cholangiocarcinoma of the liver with residual or relapsed tumor. |
| | | Patient history included cholangiocarcinoma, post-operative Budd-Chiari syndrome, |
| | | biliary ascites, hydorthorax, dehydration, malnutrition, oliguria and acute renal |
| | | failure. Previous surgeries included cholecystectomy and resection of 85% of the liver. |
| BRAINOT19 | pINCY | Library was constructed using RNA isolated from diseased brain tissue removed from the |
| | | left frontal lobe of a 27-year-old Caucasian male during a brain lobectomy. Pathology |
| | | indicated a focal deep white matter lesion, characterized by marked gliosis, |
| | | calcifications, and hemosiderin-laden macrophages, consistent with a remote perinatal |
| | | injury. This tissue also showed mild to moderate generalized gliosis, predominantly |
| | | subpial and subcortical, consistent with chronic seizure disorder. The left temporal |
| | | lobe, including the mesial temporal structures, showed focal, marked pyramidal cell loss |
| | | and gliosis in hippocampal sector CAl, consistent with mesial temporal sclerosis. GFAP |
| | | was positive for astrocytes. The patient presented with intractable epilepsy, focal |
| | | epilepsy, hemiplegia, and an unspecified brain injury. Patient history included cerebral |
| | | palsy, abnormality of gait, and depressive disorder. Family history included brain |
| | | cancer. |
| BRAITUT12 | pINCY | Library was constructed using RNA isolated from brain tumor tissue removed from the left |
| | | frontal lobe of a 40-year-old Caucasian female during excision of a cerebral meningeal |
| | | lesion. Pathology indicated grade 4 gemistocytic astrocytoma. |
| BRAQTDR02 | PCDNA2.1 | This random primed library was constructed using RNA isolated from mixed midbrain |
| | | tissues (nucleus raphe magnus, periaqueductal gray and substantia nigra) removed from a |
| | | 55-year-old Caucasian female who died from cholangiocarcinoma. Pathology indicated mild |
| | | meningeal fibrosis predominately over the convexities, scattered axonal spheroids in the |
| | | white matter of the cingulate cortex and the thalamus, and a few scattered |
| | | neurofibrillary tangles in the entorhinal cortex and the periaqueductal gray region. |
| | | Pathology for the associated tumor tissue indicated well-differentiated |
| | | cholangiocarcinoma of the liver with residual or relapsed tumor. Patient history |
| | | included cholangiocarcinoma, post-operative Budd-Chiari syndrome, biliary ascites, |
| | | hydorthorax, dehydration, malnutrition, oliguria and acute renal failure. Previous |
| | | surgeries included cholecystectomy and resection of 85% of the liver. |
| BRSTNOT13 | pINCY | Library was constructed using RNA isolated from breast tissue removed from a 36-year-old |
| | | Caucasian female during bilateral simple mastectomy. Patient history included a breast |
| | | neoplasm, depressive disorder, hyperlipidemia, and a chronic stomach ulcer. Family |
| | | history included a cardiovascular and cerebrovascular disease; hyperlipidemia; skin, |
| | | breast, esophageal, bladder, and bone cancer; and Hodgkin's lymphoma. |
| HNT2AGT01 | PBLUESCRIPT | Library was constructed at Stratagene (STR937233), using RNA isolated from the hNT2 cell |
| | | line derived from a human teratocarcinoma that exhibited properties characteristic of a |
| | | committed neuronal precursor. Cells were treated with retinoic acid for 5 weeks and with |
| | | mitotic inhibitors for two weeks and allowed to mature for an additional 4 weeks in |
| | | conditioned medium. |
| OVARDIR01 | PCDNA2.1 | This random primed library was censtructed using RNA isolated from right ovary tissue |
| | | removed from a 45-year-old Caucasian female during total abdominal hysterectomy, |
| | | bilateral salpingo-oophorectomy, vaginal suspension and fixation, and incidental |
| | | appendectomy. Pathology indicated stromal hyperthecosis of the right and left ovaries. |
| | | Pathology for the matched tumor tissue indicated a dermoid cyst (benign cystic teratoma) |
| | | in the left ovary. Multiple (3) intramural leiomyomata were identified. The cervix |
| | | showed squamous metaplasia. Patient history included metrorrhagia, female stress |
| | | incontinence, alopecia, depressive disorder, pneumonia, normal delivery, and deficiency |
| | | anemia. Family history included benign hypertension, atherosclerotic coronary artery |
| | | disease, hyperlipidemia, and primary tuberculous complex. |
| PROSTME06 | PCDNA2.1 | This 5′ biased random primed library was constructed using RNA isolated from diseased |
| | | prostate tissue removed from a 57-year-old Caucasian male during closed prostatic |
| | | biopsy, radical prostatectomy, and regional lymph node excision. Pathology indicated |
| | | adenofibromatous hyperplasia. Pathology for the matched tumor tissue indicated |
| | | adenocarcinoma, Gleason grade 3 + 3, forming a predominant mass involving the right side |
| | | centrally. The patient presented with elevated prostate specific antigen and prostate |
| | | cancer. Patient history included tobacco abuse in remission. Previous surgeries included |
| | | cholecystectomy, repair of diaphragm hernia, and repair of vertebral fracture. Patient |
| | | medications included Pepsid, Omnipen, and Eulexin. Family history included benign |
| | | hypertension, cerebrovascular accident, atherosclerotic coronary artery disease, uterine |
| | | cancer and type II diabetes in the mother; prostate cancer in the father; drug abuse, |
| | | prostate cancer, and breast cancer in the sibling(s). |
| SINITMR01 | PCDNA2.1 | This random primed library was constructed using RNA isolated from ileumn tissue removed |
| | | from a 70-year-old Caucasian female during right hemicolectomy, open liver biopsy, |
| | | flexible sigmoidoscopy, colonoscopy, and permanent colostomy. Pathology for the matched |
| | | tumor tissue indicated invasive grade 2 adenocarcinoma forming an ulcerated mass, |
| | | situated 2 cm distal to the ileocecal valve. Patient history included a malignant breast |
| | | neoplasm, type II diabetes, hyperlipidemia, viral hepatitis, an unspecified thyroid |
| | | disorder, osteoarthritis, a malignant skin neoplasm, deficiency anemia, and normal |
| | | delivery. Family history included breast cancer, atherosclerotic coronary artery |
| | | disease, benign hypertension, cerebrovascular disease, ovarian cancer, and |
| | | hyperlipidemia. |
| SINTNOT02 | PBLUESCRIPT | Library was constructed using RNA isolated from the small intestine of a 55-year-old |
| | | Caucasian female, who died from a subarachnoid hemorrhage. Serologies were positive for |
| | | cytomegalovirus (CMV). Previous surgeries included a hysterectomy. |

TABLE 7

| Program | Description | Reference | Parameter Threshold |
|---|---|---|---|
| ABI FACTURA | A program that removes vector | Applied Biosystems, Foster City, CA. | |
| | sequences and masks ambiguous | | |
| | bases in nucleic acid sequences. | | |
| ABI/ | A Fast Data Finder useful in | Applied Biosystems, Foster City, CA; | Mismatch <50% |
| PARACEL FDF | comparing and annotating amino | Paracel Inc., Pasadena, CA. | |
| | acid or nucleic acid sequences. | | |
| ABI AutoAssembler | A program that assembles nucleic | Applied Biosystems, Foster City, CA. | |
| | acid sequences. | | |
| BLAST | A Basic Local Alignment Search | Altschul, S. F. et at. (1990) J. Mol. Biol. | ESTs: Probability value = 1.0E−8 |
| | Tool useful in sequence similarity | 215:403-410; Altschul, S. F. et at. (1997) | or less |
| | search for amino acid and | Nucleic Acids Res. 25:3389-3402. | Full Length sequences: Probability |
| | nucleic acid sequences. BLAST | | value = 1.0E−10 or less |
| | includes five functions: blasp, | | |
| | blasta, blastx, tblastn, | | |
| | and tblastx. | | |
| FASTA | A Pearson and Lipman algorithm | Pearson, W. R. and D. J. Lipman (1988) Proc. | ESTs: fasta E value = 1.06E−6 |
| | that searches for similarity | Natl. Acad Sci. USA 85:2444-2448; Pearson, | Assembled ESTs: fasta Identity = |
| | between a query sequence and | W. R. (1990) Methods Eazymol. 183:63-98; | 95% or greater and |
| | a group of sequences of the same | and Smith, T. F. and M. S. Waterman (1981) | Match length = 200 bases or |
| | type. FASTA comprises as least | Adv. Appl. Math. 2:482-489. | greater; fastx E value = |
| | five functions: fasta, tfasta, | | 1.0E−8 or less Full Length |
| | fastx, tfastx, and ssearch. | | sequences: fastx score = 100 or |
| | | | greater |
| BLIMPS | A BLocks IMProved Searcher that | Henikoff, S. and J. G. Henikoff (1991) Nucleic | Probability value = 1.0E−3 or less |
| | matches a sequence against those | Acids Res. 19:6565-6572; Henikoff, J. G. and | |
| | in BLOCKS, PRINTS, DOMO, | S. Henikoff (1996) Methods Enzymol. | |
| | PRODOM, and PFAM databases | 266:88-105; and Attwood, T. K. et al. (1997) J. | |
| | to search for gene families, | Chem. Inf. Comput. Sci. 37:417-424. | |
| | sequence homology, and structural | | |
| | fingerprint regions. | | |
| HMMER | An algorithm for searching a query | Krogh, A. et al. (1994) J. Mol. Biol. | PFAM hits: Probability value = |
| | sequence against hidden Markov | 235:1501-1531; Sonnhammer, E. L. L. et al. | 1.0E−3 or less |
| | model (HMM)-based databases of | (1988) Nucleic Acids Res. 26:320-322; | Signal peptide hits: Score = 0 or |
| | protein family consensus | Durbin, R. et al. (1998) Our World View, in a | greater |
| | sequences, such as PFAM. | Nutshell, Cambridge Univ. Press, pp. 1-350. | |
| ProfileScan | An algorithm that searches for | Gribskov, M. et al. (1988) CABIOS 4:61-66; | Normalized quality score ≧ GCG- |
| | structural and sequence motifs in | Gribskov, M. et al. (1989) Methods Enzymol. | specified “HIGH” value for that |
| | protein sequences that match | 183:146-159; Bairoch, A. et al. (1997) | particular Prosite motif. |
| | sequence patterns defined in | Nucleic Acids Res. 25:217-221. | Generally, score = 1.4-2.1. |
| | Prosite. | | |
| Phred | A base-calling algorithm that | Ewing, B. et al. (1998) Genome Res. | |
| | examines automated sequencer | 8:175-185; Ewing, B. and P. Green | |
| | traces with high sensitivity | (1998) GenomeRes. 8:186-194. | |
| | and probability. | | |
| Phrap | A Phils Revised Assembly | Smith, T. F. and M. S. Waterman (1981) Adv. | Score = 120 or greater; |
| | Program including SWAT and | Appl. Math. 2:482-489; Smith, T. F. and M. S. | Match length = 56 or greater |
| | CrossMatch, programs based on | Waterman (1981) J. Mol. Biol. 147:195-197; | |
| | efficient implementation of the | and Green, P., University of Washington, | |
| | Smith-Waterman algorithm, useful | Seattle, WA. | |
| | in searching sequence homology | | |
| | and assembling DNA sequences. | | |
| Consed | A graphical tool for viewing and | Gordon, D. et al. (1998) Genome Res. 8:195-202. | |
| | editing Phrap assemblies. | | |
| SPScan | A weight matrix analysis program | Nielson, H. et al. (1997) Protein Engineering | Score = 3.5 or greater |
| | that scans protein sequences for | 10:1-6; Claverie, J. M. and S. Audic (1997) | |
| | the presence of secretory signal | CABIOS 12:431-439. | |
| | peptides. | | |
| TMAP | A program that uses weight | Persson, B. and P. Argos (1994) J. Mol. Biol. | |
| | matrices to delineate trans- | 237:182-192; Persson, B. and P. Argos (1996) | |
| | membrane segments on protein | Protein Sci. 5:363-371. | |
| | sequences and determine | | |
| | orientation. | | |
| TMHMMER | A program that uses a hidden | Sonnhammer, E. L. et al. (1998) Proc. Sixth Intl. | |
| | Markov model (HMM) to delineate | Conf. on Intelligent Systems for Mol. Biol., | |
| | transmembrane segments on | Glasgow et al., eds., The Am. Assoc. for Artificial | |
| | protein sequences and determine | Intelligence Press, Menlo Park, CA, pp. 175-182. | |
| | orientation. | | |
| Motifs | A program that searches amino | Bairoch, A. et al. (1997) Nucleic Acids Res. 25:217-221; | |
| | acid sequences for patterns | Wisconsin Package Program Manual, version 9, page | |
| | that matched those defined in | M51-59, Genetics Computer Group, Madison, WI. | |
| | Prosite. | | |

What is claimed is:

1. An isolated polypeptide selected from the group consisting of:
a) a polypeptide comprising an amino acid sequence selected from the group consisting of SEQ ID NO:1-19,
b) a polypeptide comprising a naturally occurring amino acid sequence at least 90% identical to an amino acid sequence selected from the group consisting of SEQ ID NO:1-19,
c) a biologically active fragment of a polypeptide having an amino acid sequence selected from the group consisting of SEQ ID NO:1-19, and
d) an immunogenic fragment of a polypeptide having an amino acid sequence selected from the group consisting of SEQ ID NO:1-19.

2. An isolated polypeptide of claim 1 selected from the group consisting of SEQ ID NO:1-19.

3. An isolated polynucleotide encoding a polypeptide of claim 1.

4. An isolated polynucleotide encoding a polypeptide of claim 2.

5. An isolated polynucleotide of claim 4 selected from the group consisting of SEQ ID NO:20-38.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably lined to a polynucleotide encoding the polypeptide of claim 1, and
b) recovering the polypeptide so expressed.

10. An is lated antibody which specifically binds to a polypeptide of claim 1.

11. An isolated polynucleotide selected from the group consisting of:
a) a polynucleotide comprising a polynucleotide sequence selected from the group consisting of SEQ ID NO:20-38,
b) a polynucleotide comprising a naturally occurring polynucleotide sequence at least 90% identical to a polynucleotide sequence selected from the group consisting of SEQ ID NO:20-38,
c) a polynucleotide complementary to a polynucleotide of a),
d) a polynucleotide complementary to a polynucleotide of b), and
e) an RNA equivalent of a)-d).

12. An isolated polynucleotide comprising at least 60 contiguous nucleotides of a polynucleotide of claim 11.

13. A method of detecting a target polynucleotide in a sample, said target polynucleotide having a sequence of a polynucleotide of claim 11, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

14. A method of claim 13, wherein the probe comprises at least 60 contiguous nucleotides.

15. A method of detecting a target polynucleotide in a sample, said target polynucleotide having a sequence of a polynucleotide of claim 11, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

16. A composition comprising a polypeptide of claim 1 and a pharmaceutically acceptable excipient.

17. A composition of claim 16, wherein the polypeptide has an amino acid sequence selected from the group consisting of SEQ ID NO:1-19.

18. A method for treating a disease or condition associated with decreased expression of functional DME, comprising administering to a patient in need of such treatment the composition of claim 16.

19. A method of screening a compound for effectiveness as an agonist of a polypeptide of claim 1, the method comprising:
a) exposing a sample comprising a polypeptide of claim 1 to a compound, and
b) detecting agonist activity in the sample.

20. A composition comprising an agonist compound identified by a method of claim 19 and a pharmaceutically acceptable excipient.

21. A method for treating a disease or condition associated with decreased expression of functional DME, comprising administering to a patient in need of such treatment a composition of claim 20.

22. A method of screening a compound for effectiveness as an antagonist of a polypeptide of claim 1, the method comprising:
a) exposing a sample comprising a polypeptide of claim 1 to a compound, and
b) detecting antagonist activity in the sample.

23. A composition comprising an antagonist compound identified by a method of claim 22 and a pharmaceutically acceptable excipient.

24. A method for treating a disease or condition associated with overexpression of functional DME, comprising administering to a patient in need of such treatment a composition of claim 23.

25. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

26. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

27. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

28. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 11 under conditions whereby a specific hybridization complex is formed between said probe and a target polynucleotide in the biological sample, said target polynucleotide comprising a polynucleotide sequence of a polynucleotide of claim 11 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

29. A diagnostic test for a condition or disease associated with the expression of DME in a biological sample, the method comprising:
a) combining the biological sample with an antibody of claim 10, under conditions suitable for the antibody to bind the polypeptide and form an antibody:polypeptide complex, and
b) detecting the complex, wherein the presence of the complex correlates with the presence of the polypeptide in the biological sample.

30. The antibody of claim 10, wherein the antibody is:
a) a chimeric antibody,
b) a single chain antibody,
c) a Fab fragment,
d) a F(ab′)₂ fragment, or
e) a humanized antibody.

31. A composition comprising an antibody of claim 10 and an acceptable excipient.

32. A method of diagnosing a condition or disease associated with the expression of DME in a subject, comprising administering to said subject an effective amount of the composition of claim 31.

33. A composition of claim 31, wherein the antibody is labeled.

34. A method of diagnosing a condition or disease associated with the expression of DME in a subject, comprising administering to said subject an effective amount of the composition of claim 33.

35. A method of preparing a polyclonal antibody with the specificity of the antibody of claim 10, the method comprising:
a) immunizing an animal with a polypeptide having an amino acid sequence selected from the group consisting of SEQ ID NO:1-19, or an immunogenic fragment thereof, under conditions to elicit an antibody response,
b) isolating antibodies from said animal, and
c) screening the isolated antibodies with the polypeptide, thereby identify a polyclonal antibody which binds specifically to a polypeptide having an amino acid sequence selected from the group consisting of SEQ ID NO:1-19.

36. An antibody produced by a method of claim 35.

37. A composition comprising the antibody of claim 36 and a suitable carrier.

38. A method of making a monoclonal antibody with the specificity of the antibody of claim 10, the method comprising:
a) immunizing an animal with a polypeptide having an amino acid sequence selected from the group consisting of SEQ ]D NO:1-19, or an immunogenic fragment thereof, under conditions to elicit an antibody response,
b) isolating antibody producing cells from the animal,
c) fusing the antibody producing cells with immortalized cells to form monoclonal antibody-producing hybridoma cells,
d) culturing the hybridoma cells, and
e) isolating from the culture monoclonal antibody which binds specifically to a polypeptide having an amino acid sequence selected from the group consisting of SEQ ID NO:1-19.

39. A monoclonal antibody produced by a method of claim 38.

40. A composition comprising the antibody of claim 39 and a suitable carrier.

41. The antibody of claim 10, wherein the antibody is produced by screening a Fab expression library.

42. The antibody of claim 10, wherein the antibody is produced by screening a recombinant immunoglobulin library.

43. A method of detecting a polypeptide having an amino acid sequence selected from the group consisting of SEQ ID NO:1-19 in a sample, the method comprising:
a) incubating the antibody of claim 10 with a sample under conditions to allow specific binding of the antibody and the polypeptide, and
b) detecting specific binding, wherein specific binding indicates the presence of a polypeptide having an amino acid sequence selected from the group consisting of SEQ ID NO:1-19 in the sample.

44. A method of purifying a polypeptide having an amino acid sequence selected from the group consisting of SEQ ID NO:1-19 from a sample, the method comprising:
a) incubating the antibody of claim 10 with a sample under conditions to allow specific binding of the antibody and the polypeptide, and
b) separating the antibody from the sample and obtaining the purified polypeptide having an amino acid sequence selected from the group consisting of SEQ ID NO:1-19.

45. A polypeptide of claim 1, comprising the amino acid sequence of SEQ ID NO:1.

46. A polypeptide of claim 1, comprising the amino acid sequence of SEQ ID NO:2.

47. A polypeptide of claim 1, comprising the amino acid sequence of SEQ ID NO:3.

48. A polypeptide of claim 1, comprising the amino acid sequence of SEQ ID NO:4.

49. A polypeptide of claim 1, comprising the amino acid sequence of SEQ ID NO:5.

50. A polypeptide of claim 1, comprising the amino acid sequence of SEQ ID NO:6.

51. A polypeptide of claim 1, comprising the amino acid sequence of SEQ ID NO:7.

52. A polypeptide of claim 1, comprising the amino acid sequence of SEQ ID NO:8.

53. A polypeptide of claim 1, comprising the amino acid sequence of SEQ ID NO:9.

54. A polypeptide of claim 1, comprising the amino acid sequence of SEQ ID NO:10.

55. A polypeptide of claim 1, comprising the amino acid sequence of SEQ ID NO:11.

56. A polypeptide of claim 1, comprising the amino acid sequence of SEQ ID NO:12.

57. A polypeptide of claim 1, comprising the amino acid sequence of SEQ ID NO:13.

58. A polypeptide of claim 1, comprising the amino acid sequence of SEQ ID NO:14.

59. A polypeptide of claim 1, comprising the amino acid sequence of SEQ ID NO:15.

60. A polypeptide of claim 1, comprising the amino acid sequence of SEQ ID NO:16.

61. A polypeptide of claim 1, comprising the amino acid sequence of SEQ ID NO:17.

62. A polypeptide of claim 1, comprising the amino acid sequence of SEQ ID NO:18.

63. A polypeptide of claim 1, comprising the amino acid sequence of SEQ ID NO:19.

64. A polynucleotide of claim 11, comprising the polynucleotide sequence of SEQ ID NO:20.

65. A polynucleotide of claim 11, comprising the polynucleotide sequence of SEQ ID NO:21.

66. A polynucleotide of claim 11, comprising the polynucleotide sequence of SEQ ID NO:22.

67. A polynucleotide of claim 11, comprising the polynucleotide sequence of SEQ ID NO:23.

68. A polynucleotide of claim 11, comprising the polynucleotide sequence of SEQ ID NO:24.

69. A polynucleotide of claim 11, comprising the polynucleotide sequence of SEQ ID NO:25.

70. A polynucleotide of claim 11, comprising the polynucleotide sequence of SEQ ID NO:26.

71. A polynucleotide of claim 11, comprising the polynucleotide sequence of SEQ ID NO:27.

72. A polynucleotide of claim 11, comprising the polynucleotide sequence of SEQ ID NO:28.

73. A polynucleotide of claim 11, comprising the polynucleotide sequence of SEQ ID NO:29.

74. A polynucleotide of claim 11, comprising the polynucleotide sequence of SEQ ID NO:30.

75. A polynucleotide of claim 11, comprising the polynucleotide sequence of SEQ ID NO:31.

76. A polynucleotide of claim 11, comprising the polynucleotide sequence of SEQ ID NO:32.

77. A polynucleotide of claim 11, comprising the polynucleotide sequence of SEQ ID NO:33.

78. A polynucleotide of claim 11, comprising the polynucleotide sequence of SEQ ID NO:34.

79. A polynucleotide of claim 11, comprising the polynucleotide sequence of SEQ ID NO:35.

80. A polynucleotide of claim 11, comprising the polynucleotide sequence of SEQ ID NO:36.

81. A polynucleotide of claim 11, comprising the polynucleotide sequence of SEQ ID NO:37.

82. A polynucleotide of claim 11, comprising the polynucleotide sequence of SEQ ID NO:38.

83. A method of claim 9, wherein the polypeptide has the sequence of SEQ ID NO:1.

84. A method of claim 9, wherein the polypeptide has the sequence of SEQ ID NO:2.

85. A method of claim 9, wherein the polypeptide has the sequence of SEQ ID NO:3.

86. A method of claim 9, wherein the polypeptide has the sequence of SEQ ID NO:4.

87. A method of claim 9, wherein the polypeptide has the sequence of SEQ ID NO:5.

88. A method of claim 9, wherein the polypeptide has the sequence of SEQ ID NO:6.

89. A method of claim 9, wherein the polypeptide has the sequence of SEQ ID NO:7.

90. A method of claim 9, wherein the polypeptide has the sequence of SEQ ID NO:8.

91. A method of claim 9, wherein the polypeptide has the sequence of SEQ ID NO:9.

92. A method of claim 9, wherein the polypeptide has the sequence of SEQ ID NO:10.

93. A method of claim 9, wherein the polypeptide has the sequence of SEQ ID NO:11.

94. A method of claim 9, wherein the polypeptide has the sequence of SEQ ID NO:12.

95. A method of claim 9, wherein the polypeptide has the sequence of SEQ ID NO:13.

96. A method of claim 9, wherein the polypeptide has the sequence of SEQ ID NO:14.

97. A method of claim 9, wherein the polypeptide has the sequence of SEQ ID NO:15.

98. A method of claim 9, wherein the polypeptide has the sequence of SEQ ID NO:16.

99. A method of claim 9, wherein the polypeptide has the sequence of SEQ ID NO:17.

100. A method of claim 9, wherein the polypeptide has the sequence of SEQ ID NO:18.

101. A method of claim 9, wherein the polypeptide has the sequence of SEQ ID NO:19.

102. A microarray wherein at least one element of the microarray is a polynucleotide of claim 12.

103. A method for generating a transcript image of a sample which contains polynucleotides, the method comprising the steps of:
a) labeling the polynucleotides of the sample,
b) contacting the elements of the microarray of claim 102 with the labeled polynucleotides of the sample under conditions suitable for the formation of a hybridization complex, and
c) quantifying the expression of the polynucleotides in the sample.

104. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, said target polynucleotide having a sequence of claim 11.

105. An array of claim 104, wherein said first oligonucleotide or polynucleotide sequence is completely complementary to at least 30 contiguous nucleotides of said target polynucleotide.

106. An array of claim 104, wherein said first oligonucleotide or polynucleotide sequence is completely complementary to at least 60 contiguous nucleotides of said target polynucleotide.

107. An array of claim 104, which is a microarray.

108. An array of claim 104, further comprising said target polynucleotide hybridized to said first oligonucleotide or polynucleotide.

109. An array of claim 104, wherein a linker joins at least one of said nucleotide molecules to said solid substrate.

110. An array of claim 104, wherein each distinct physical location on the substrate contains multiple nucleotide molecules having the same sequence, and each distinct physical location on the substrate contains nucleotide molecules having a sequence which differs from the sequence of nucleotide molecules at another physical location on the substrate.
